(19) United States
(12) Patent Application Publication
(10) Pub. No.: US 2026/0311826 A1
(43) Pub. Date: Oct. 8, 2026

(54) SYSTEM AND METHOD FOR OPTIMIZING CLINICAL AND OPERATIONAL WORKFLOWS IN HEALTHCARE ENVIRONMENTS

(71) Applicant: Verentia Innovations, LLC, New York, NY (US)

(72) Inventor: Kevin A. DiMauro, New York, NY (US)

(21) Appl. No.: 19/456,729

(22) Filed: Jan. 22, 2026

Related U.S. Application Data
(60) Provisional application No. 63/748,428, filed on Jan. 22, 2025.
(60) Provisional application No. 63/803,622, filed on May 10, 2025.
(60) Provisional application No. 63/914,002, filed on Nov. 8, 2025.

(51) Int. Cl. G16H 10/60; G16H 40/40

(52) U.S. Cl. CPC G16H 10/60; G16H 40/40

(57) ABSTRACT

System and method for optimizing clinical and operational workflows in healthcare environments. The method includes receiving data indicating resources and patient records, generating de-identified feature representations excluding direct patient identifiers, and constructing a model based on the de-identified feature representations, resource data, and patient records. Weights are derived from the model to represent resource availability under constraints, and resources are assigned to patient records by solving an objective function based on the derived weights. The assigned resources and corresponding time intervals are recorded in a log associated with health events and outputted to authorized users within a secure user interface under role-based access control.

### CROSS-REFERENCE TO RELATED APPLICATION

This application claims the benefit of U.S. Provisional Application Nos. 63/748,428 filed Jan. 22, 2025, titled “SurgiSyncAI—AI-Driven Real-Time Surgical Scheduling and Procedural Optimization System”, U.S. 63/803,622 filed May 10, 2025, titled “SurgiSyncAI: AI-Driven Real-Time Surgical Scheduling and Procedural Optimization System”, U.S. 63/914,002 Filed Nov. 8, 2025, titled “Privacy-Preserving De-Identification and UI Boundary Architecture”, the entire disclosures of which is incorporated by reference herein.

### TECHNICAL FIELD

The present disclosure relates to healthcare information technology, and in particular, to artificial intelligence-assisted systems and methods for optimizing clinical and operational workflows in healthcare environments.

### BACKGROUND

Healthcare facilities routinely coordinate the use of limited resources, such as operating rooms, clinical staff, specialized equipment, and inpatient beds, across multiple units and service lines under rapidly changing conditions. These institutions typically depend on static block schedules, whiteboards, and ad hoc spreadsheet models that require manual updates and offer little flexibility for accommodating add-on cases, emergent changes, or downstream capacity pressures.

However, many software tools exist to address narrow parts of the problem, including block utilization analytics or heuristic support for elective case scheduling, but these systems are not designed to integrate real-time operational insights, discharge planning data, and census forecasts within a single workflow. This fragmentation often results in hospitals experiencing scheduling conflicts, resource underutilization, and increased administrative burden, underscoring the need for more cohesive, adaptive solutions.

Additionally, the handling of protected health information (PHI) in healthcare environments is required to comply with regulations such as HIPAA, GDPR, and institutional policies. Existing systems may lack clear boundaries between PHI handling, model computation, and user-facing outputs, making the process of ensuring compliance, auditability, and governance in the use of advanced information technology solutions more challenging.

### SUMMARY

In one embodiment, a computer-implemented method for optimizing clinical and operational workflows in healthcare environments includes receiving data indicating available resources and patient records, generating for each record a de-identified feature representation that excludes direct patient identifiers, and constructing a predictive model based on those representations, the patient records, and the resource data. The method further involves deriving a set of weights from the model that quantify resource availability over time under defined constraints, assigning resources to each record by solving an objective function using the derived weights, recording the assigned resources and corresponding time intervals in a health-event log, and outputting that log to authorized users. Additionally, the method may include generating a conflict graph to represent incompatibilities among records, applying credential checks on staff, readiness signals on equipment, and capacity constraints on rooms to compute weights, producing bed masks by predicting bed type, length of stay, and forecasting occupancy and capacity, computing throughput risk scores by fusing clinical urgency, financial efficiency, and fairness features with policy-tunable weights, creating de-identified identifiers via a secure de-identification server, and calculating discharge opportunity scores with safety masks to determine patient discharges.

In another embodiment, a system for optimizing workflows in a constrained operational environment comprises a processor and memory storing instructions that implement a de-identified compute plane. The system is configured to generate deterministic de-identified linkage identifiers using a keyed cryptographic function, create de-identified state representations by normalizing records, aligning timestamps, removing direct identifiers, and transforming fields into feature values, compute a set of candidate actions, evaluate feasibility conditions based on resource availability, timing constraints, and policy limits to form an admissibility mask, filter out infeasible actions, apply policy-tunable weighting parameters and select actions via a constrained optimization or reinforcement learning process, record an auditable decision record capturing the admissibility mask and policy version, update feasibility conditions or model parameters based on observed outcomes, and output executable directives or recommendations through an interface that permits controlled re-identification only within a secure enclave. Moreover, the system may enforce infeasibility as a strict constraint, support resources such as rooms, staff, equipment, and beds, maintain an identity-separated architecture with a controlled re-identification enclave under role-based access control, record re-identification events, and define admissibility over discrete time intervals.

In another embodiment, a non-transitory computer-readable medium stores instructions that, when executed by a processor, cause the processor to receive resource and patient record data, generate de-identified record representations, construct a model, derive resource availability weights, assign resources via an objective function, record assignments in a health-event log, and output the log to authorized users through a secure, role-based interface. Additionally, the instructions may permit inclusion of direct patient identifiers in the output log and support management of rooms, staff, equipment, and beds by applying credential checks, readiness signals, and capacity constraints.

### BRIEF DESCRIPTION OF THE DRAWINGS

FIG. 1A illustrates a system diagram of a conflict graph encoding surgical mutual restrictions, according to an embodiment.

FIG. 1B illustrates a process diagram of a de-identification and feature extraction pipeline for data ingestion and processing, according to an embodiment.

FIG. 1C illustrates a flowchart of a mixed-integer linear programming/reinforcement learning (MILP/RL) controller for scheduling decisions, according to an embodiment.

FIG. 2A illustrates a block diagram of a MILP assignment engine workflow integrating feasibility and credential matrices, cost generation, optimization, and output dispatch, according to an embodiment.

FIG. 2B illustrates a block diagram of a reinforcement learning policy controller, according to an embodiment.

FIG. 2C illustrates a hybrid execution loop integrating MILP outputs and RL updates, according to an embodiment.

FIG. 3A illustrates a flowchart of a Markov Decision Process (MDP) framework for action selection over a feasible set, according to an embodiment.

FIG. 3B illustrates a flowchart of a learning and convergence monitoring process, according to an embodiment.

FIG. 3C illustrates a flowchart of a policy version commit process, according to an embodiment.

FIGS. 4A-4G illustrate diagrams of a throughput risk scoring priority workflow layer, according to an embodiment.

FIG. 5A illustrates a constraint lattice for surgical and procedural scheduling that integrates feasibility masks, according to an embodiment.

FIG. 5B illustrates a process diagram of a penalty feedback engine, according to an embodiment.

FIGS. 6A-6B illustrate diagrams of a resource feasibility layer, according to an embodiment.

FIG. 7 illustrates a system diagram of a privacy-preserving de-identification architecture, according to an embodiment.

FIG. 8 illustrates a process diagram of a multi-facility routing engine workflow, according to an embodiment.

FIG. 9 illustrates a process diagram of a perioperative optimization engine, according to an embodiment.

FIG. 10 illustrates a flowchart of a coverage-aware scheduling optimization engine, according to an embodiment.

FIG. 11 illustrates a process diagram for bed and capacity assignment, according to an embodiment.

FIG. 12 illustrates a system diagram of census integration and capacity forecasting and generates LOS-aware forecasts for network-wide capacity management, according to an embodiment.

FIG. 13 illustrates a process diagram of a discharge-based optimization engine that computes discharge opportunity scores and generates bed-release events for capacity-aware scheduling, according to an embodiment.

FIG. 14 illustrates a process diagram of AI failure handling and recovery, according to an embodiment.

FIG. 15 illustrates a flowchart of a model selection and governance dashboard, according to an embodiment.

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative bases for teaching one skilled in the art to variously employ the embodiments. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical application. Various combinations and modifications of the features consistent with the teachings of this disclosure, however, could be desired for particular applications or implementations.

“A”, “an”, and “the” as used herein refers to both singular and plural referents unless the context clearly dictates otherwise. By way of example, “a processor” programmed to perform various functions refers to one processor programmed to perform each and every function, or more than one processor collectively programmed to perform each of the various functions.

The present detailed description provides illustrative embodiments of the disclosed technology, which is directed to systems and methods for privacy-preserving, auditable decision control to optimize clinical and operational workflows in healthcare environments. In various embodiments, the disclosed technology maintains an identity-separated architecture comprising an optimization compute plane that operates on deterministic linkage identifiers and de-identified feature representations, and a controlled re-identification enclave for authorized presentation of identified outputs. The disclosed technology further applies feasibility evaluation to generate feasibility masks that constrain an admissible action space, gates infeasible actions prior to optimization or learning, and selects actions using one or more computational decision techniques, including mathematical optimization and machine-learning techniques such as mixed-integer linear programming (MILP), reinforcement learning (RL), and risk-aware scoring. While the embodiments described herein focus on healthcare applications, such as surgical scheduling, perioperative workflow optimization, and hospital census forecasting, the principles and techniques disclosed may be adapted to other constrained operational environments requiring similar decision-control architectures.

Reference numerals in the figures present and the present description denote example components and interfaces. In some embodiments, functionally similar components depicted in different figures correspond to the same underlying service, subsystem, or logical module, even if illustrated using different reference numerals.

In illustrative embodiments, the disclosure distinguishes between (i) ingestion/provenance logging associated with data ingestion, feature transformation, key/version metadata, and policy configuration events, and (ii) decision/optimization logging associated with feasibility artifacts, solver outputs, selected actions, overrides, and decision provenance. Such logging may be implemented as separate ledgers or as logically partitioned streams of tamper-evident, append-only ledger service. Accordingly, references to audit ledger 130, decision ledger 138, and append-only ledger 711 may refer to distinct logical partitions and/or interfaces of a common ledger implementation, unless expressly indicated as physically separate ledgers in a given embodiment.

Likewise, the disclosure distinguishes between (i) an authoritative, versioned policy store maintaining policy versions and governance metadata, and (ii) an active policy cache or locally materialized view used at runtime to assemble feasibility artifacts and controller inputs. Accordingly, references to policy cache 132, versioned policy store 139, and policy store 710 may refer to different roles, views, or interfaces of a policy-management subsystem, with cache 132 optionally implementing a replicated or scoped view of the authoritative store 139/710, depending on embodiment.

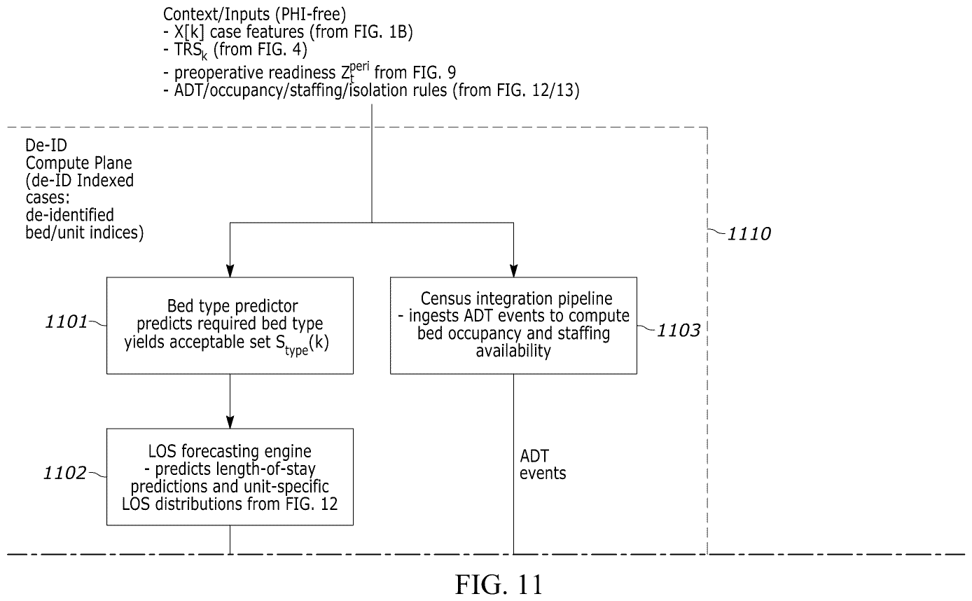

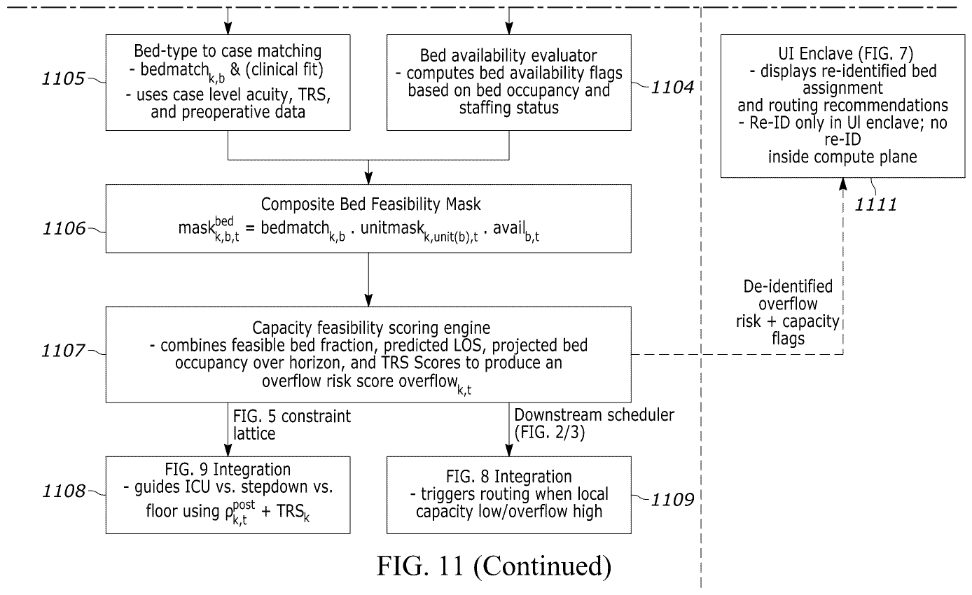

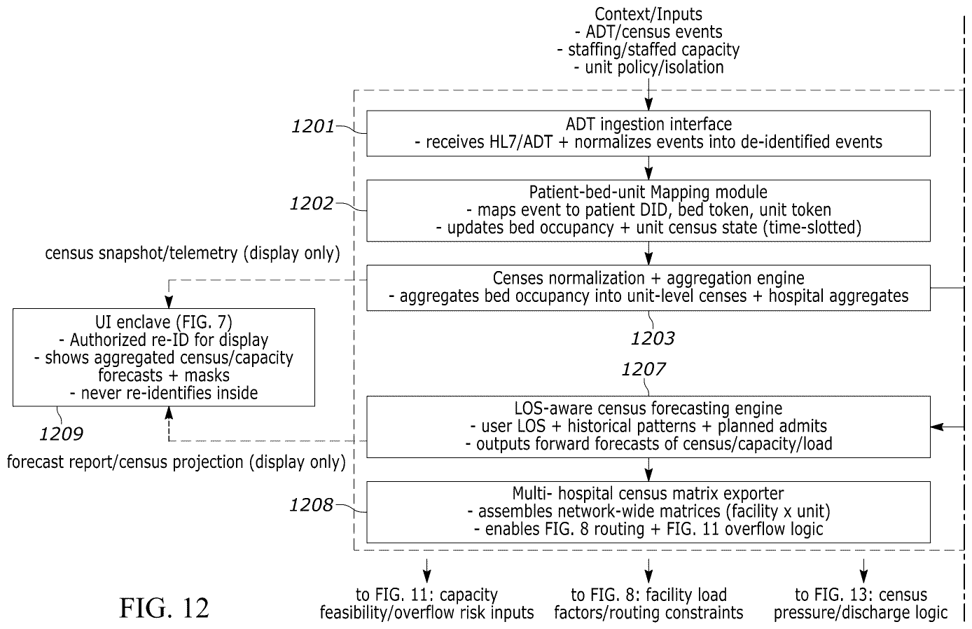

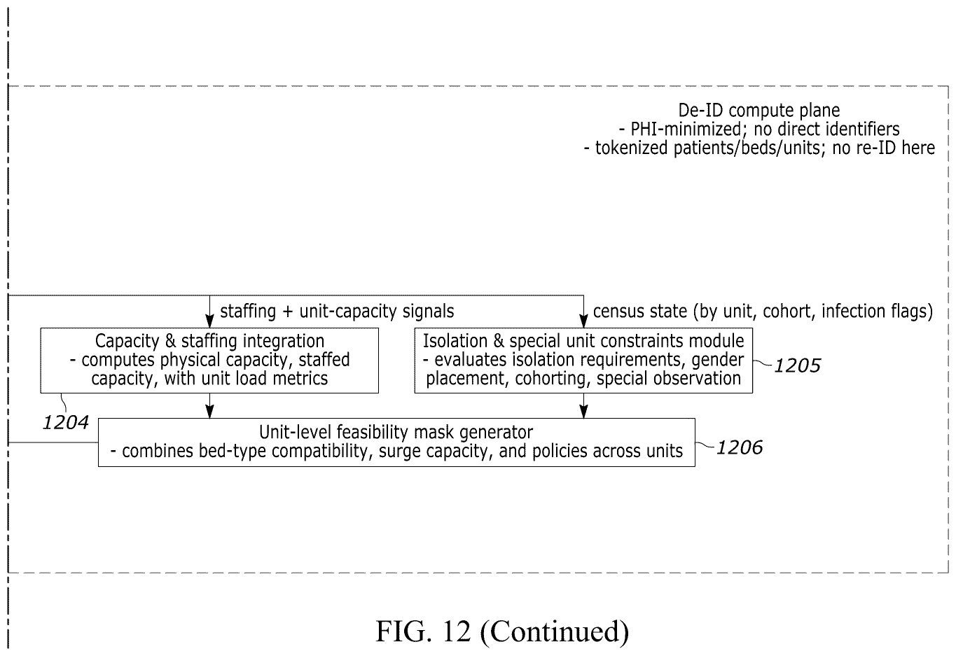

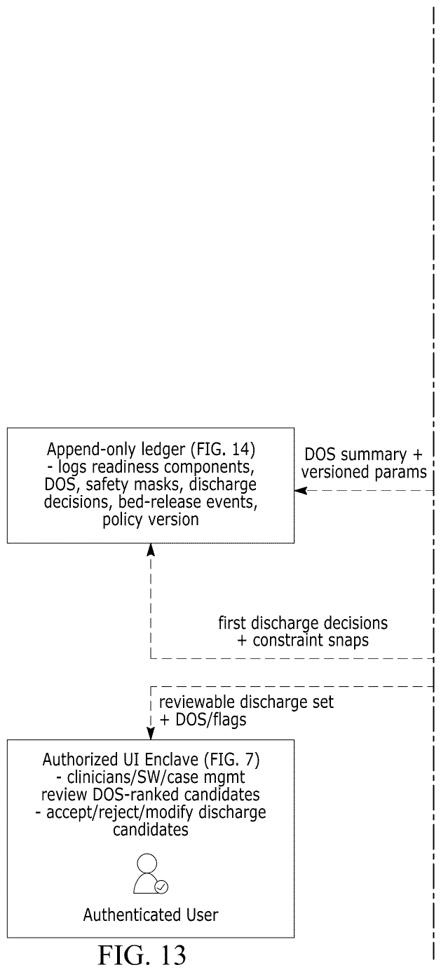

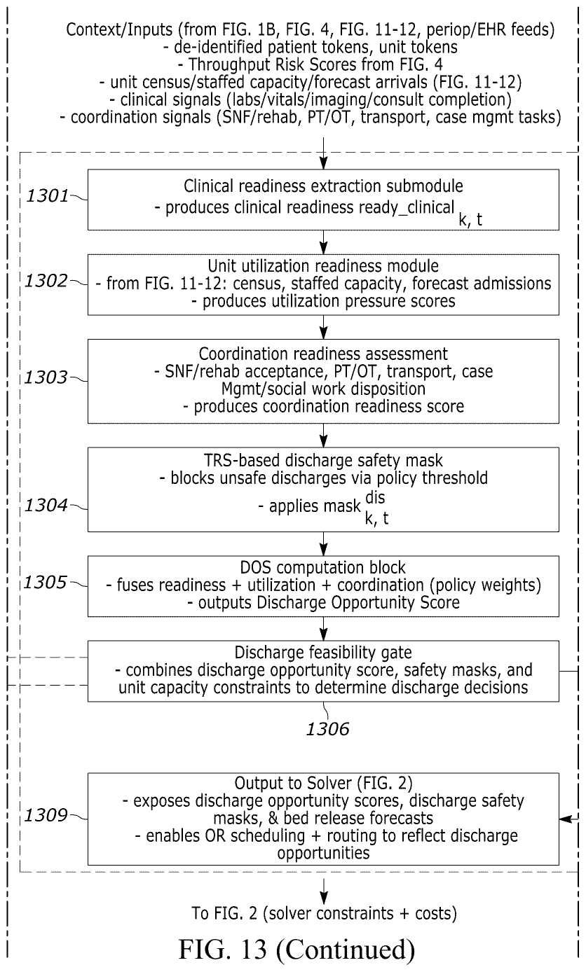

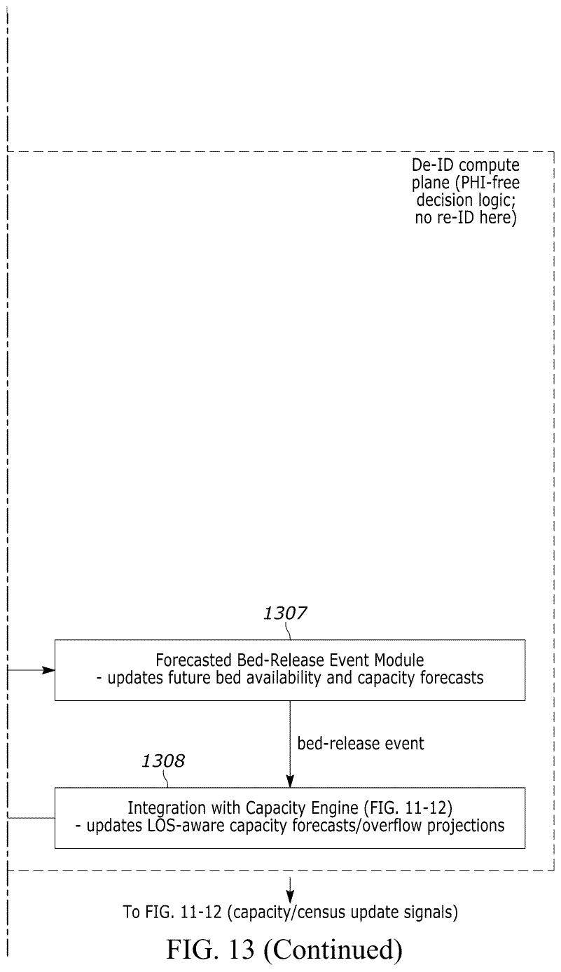

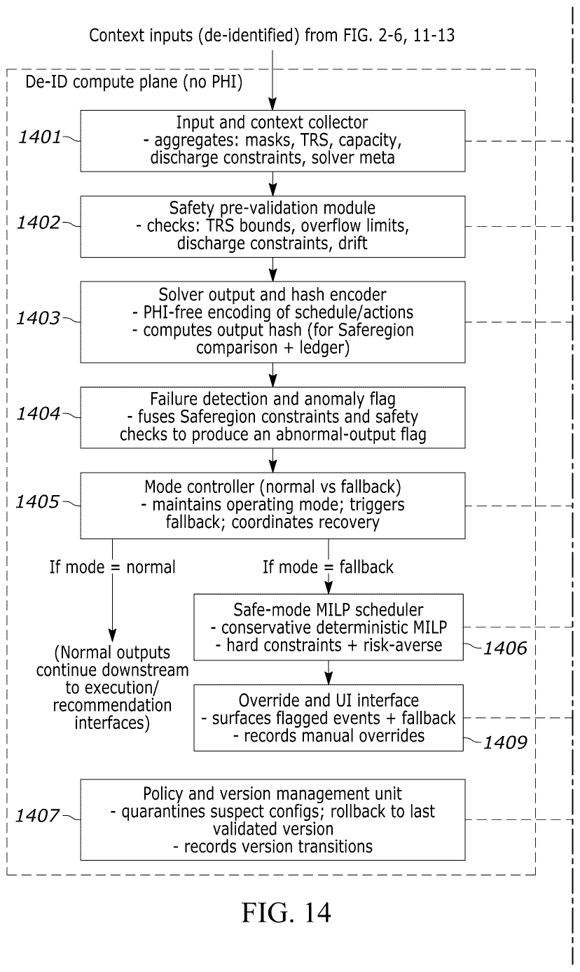

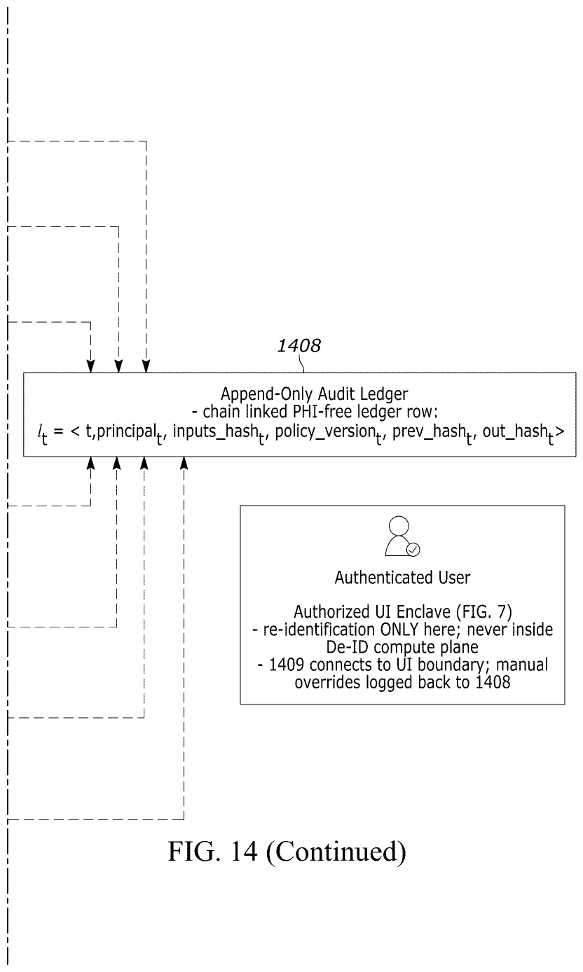

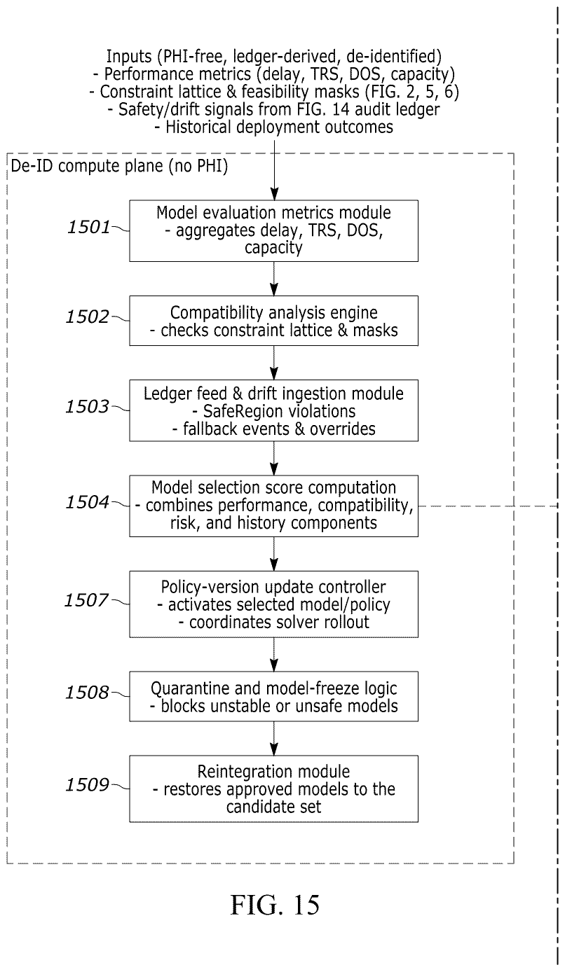

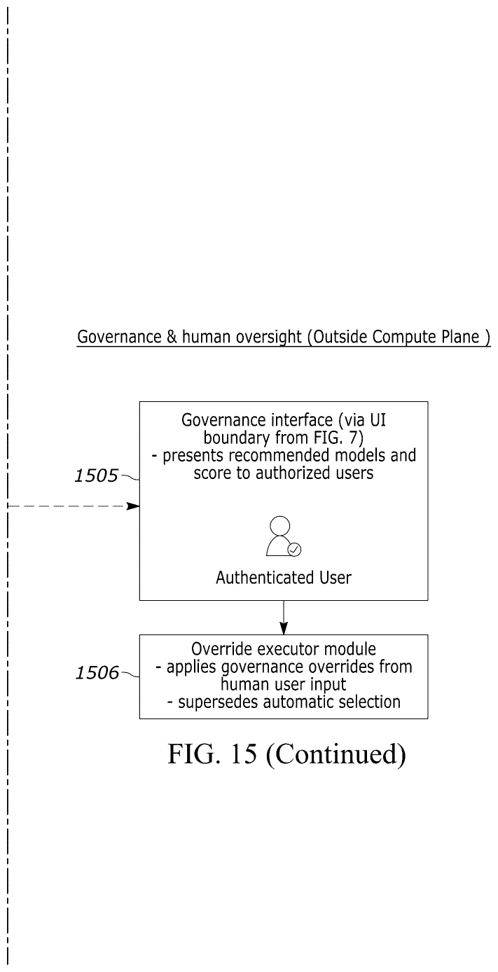

As used herein, an “optimization compute plane”, “de-identified compute plane,” or “PHI-free compute plane” refers to a logical execution boundary in which model computation, feasibility evaluation, optimization, and learning are performed on deterministic linkage identifiers and de-identified feature representations without access to direct identifiers. Multiple figures may depict this compute plane with different reference numeral to reflect different views, instances, or sub-boundaries of the same logical plane (e.g., scheduling control plane, solver plane, or mask-assembly plane). Unless expressly stated otherwise, such depictions refer to the same identity-separated compute boundary associated with the de-identification and linkage boundary (e.g., boundary 109), with re-identification confined to the controlled re-identification enclave. A dedicated example architecture for the de-identification and linkage boundary, including controlled re-identification, is illustrated in FIG. 7.

The examples provided herein are illustrative and not limiting. Details of standard data processing, software implementation, and computing infrastructure may be omitted where such details are well understood by persons of ordinary skill in the art. The disclosed subject matter encompasses modification and alternative embodiments consistent with the claims, including variations in feature transforms, scoring parameters, feasibility conditions, constraint sets, optimization or learning techniques, policy configurations, and deployment topologies, provided that the embodiments maintain feasibility-gated action selection, versioned policy governance, and tamper-evident auditability in an identity-separated architecture.

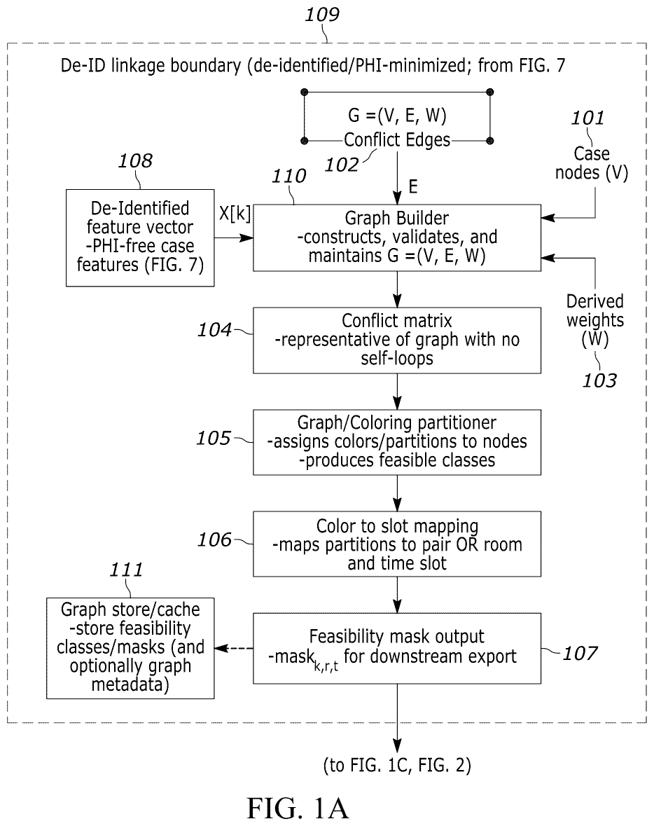

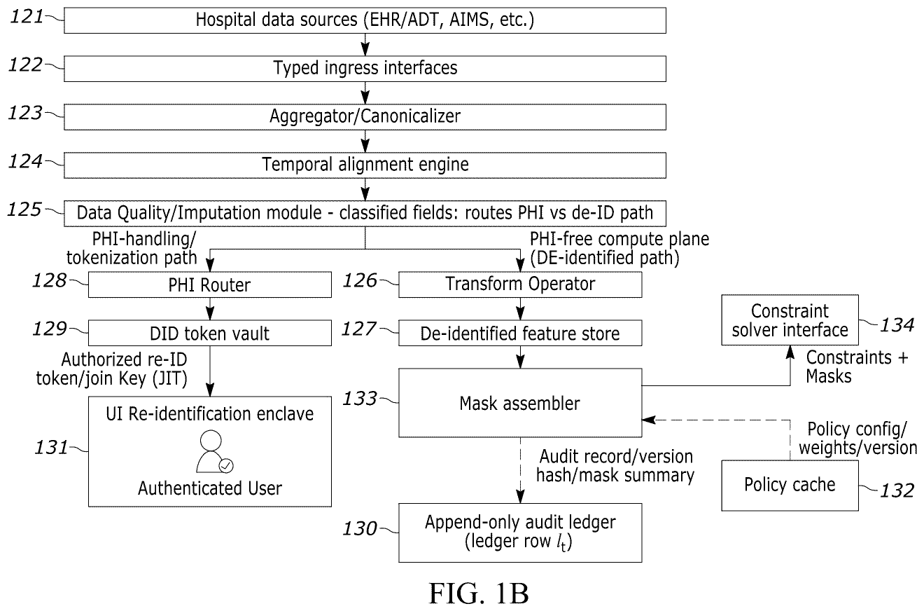

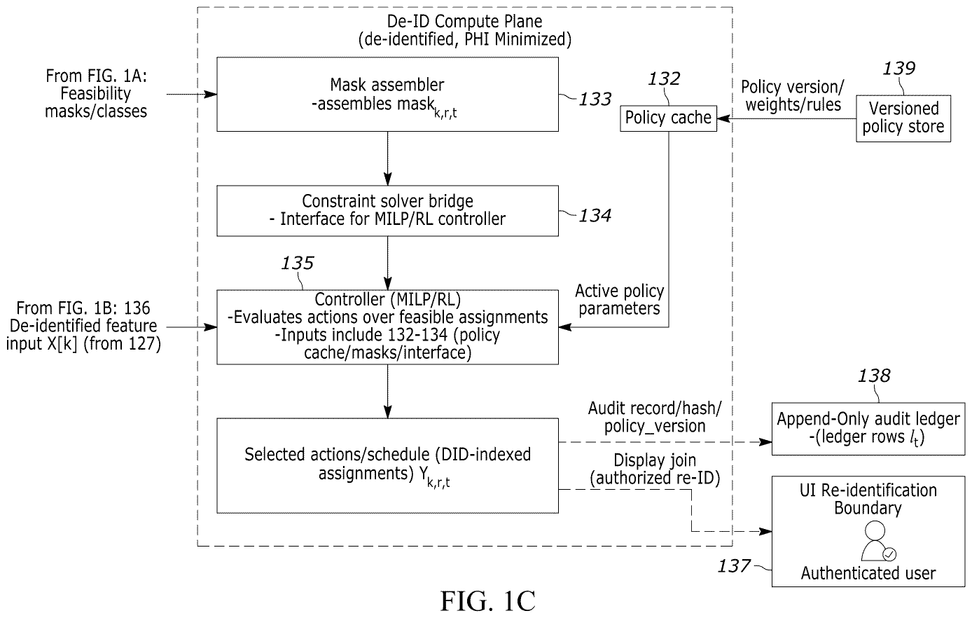

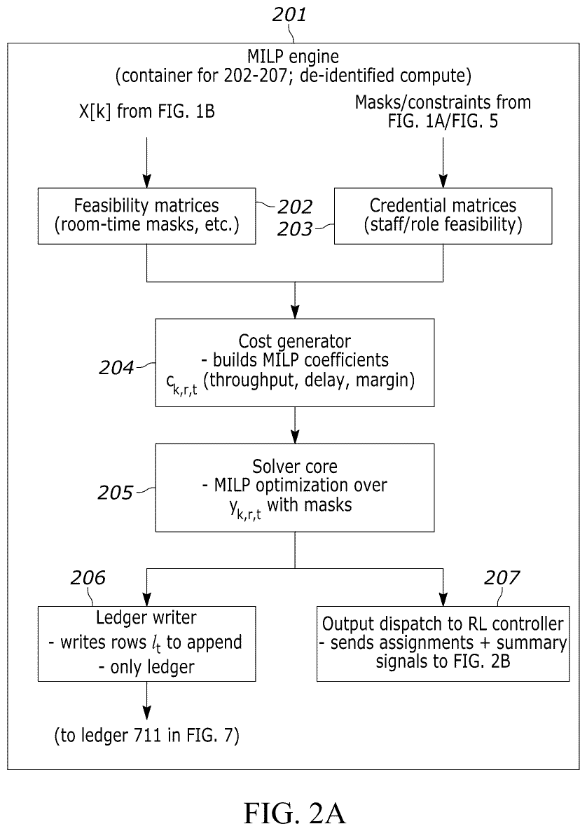

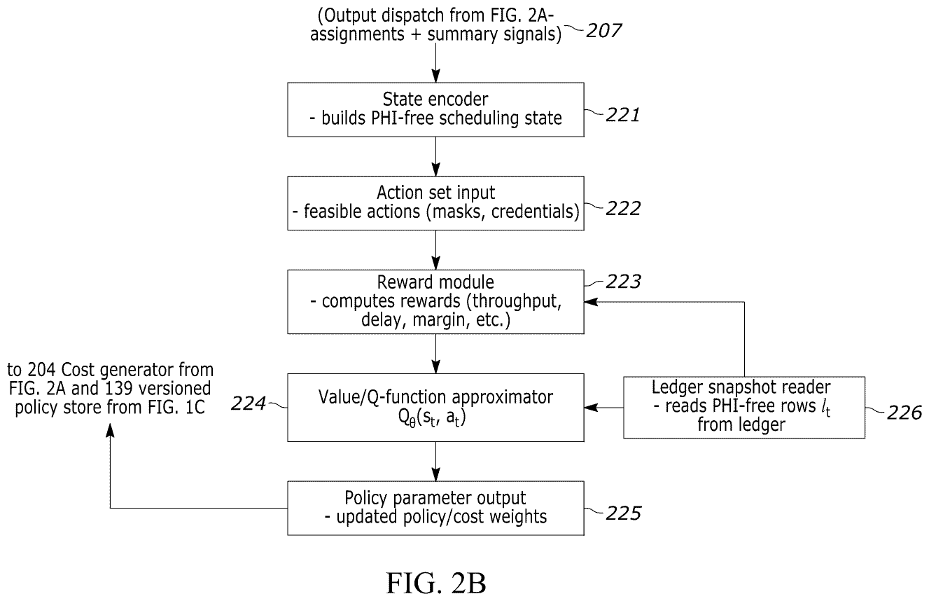

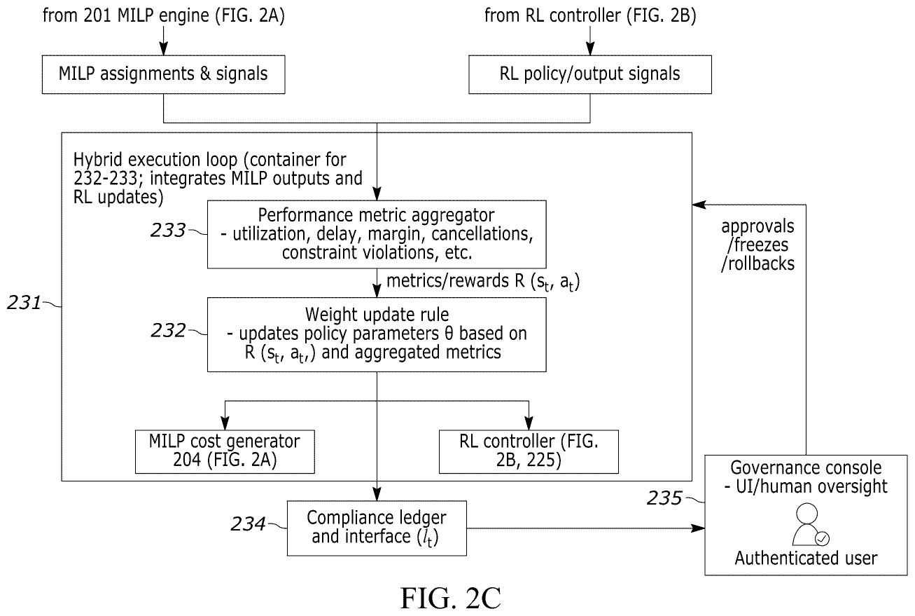

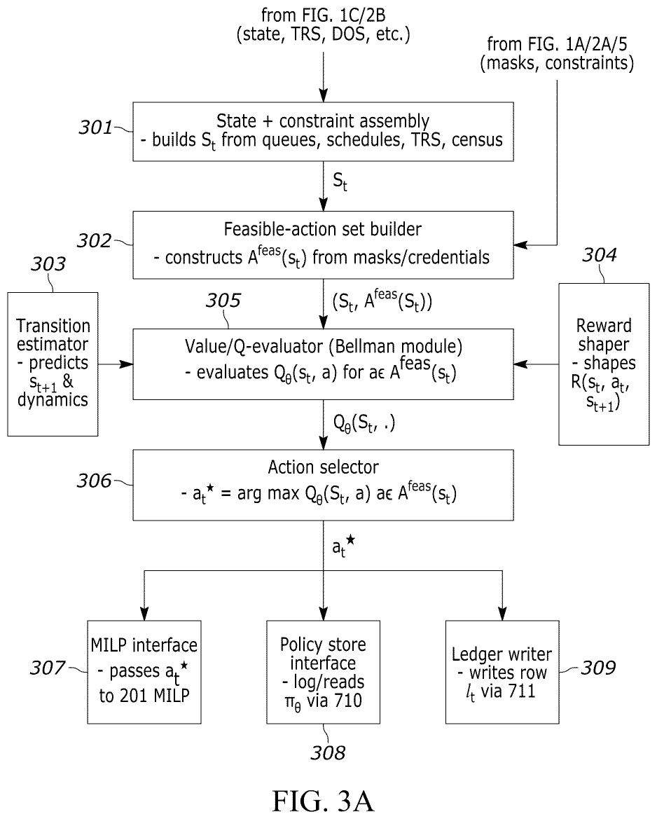

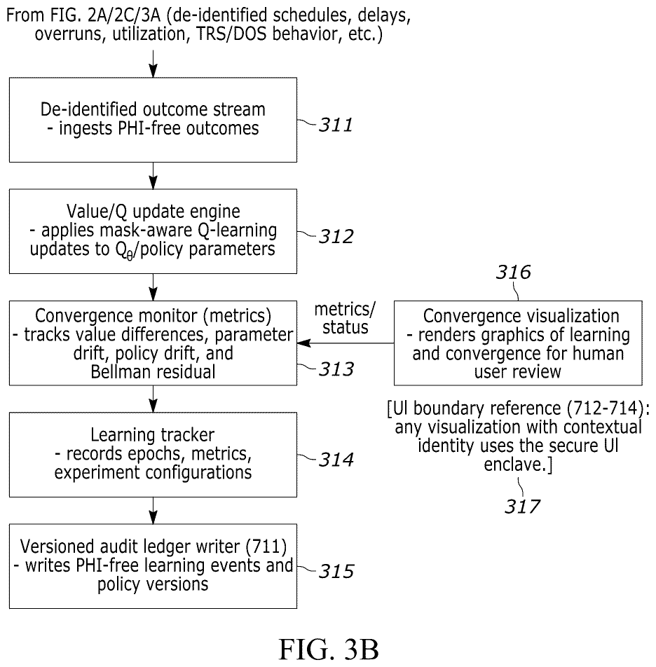

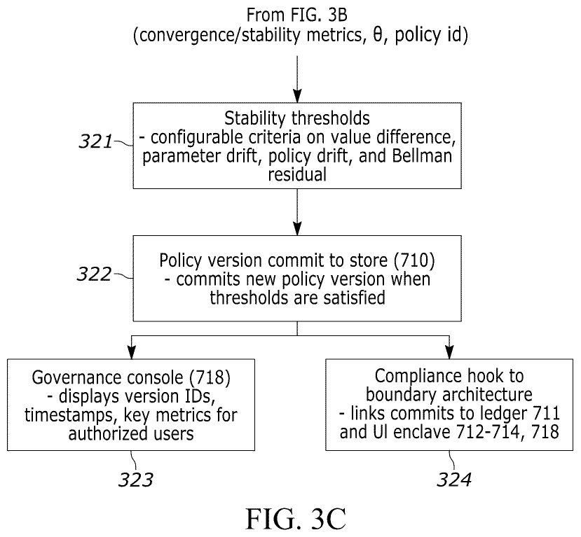

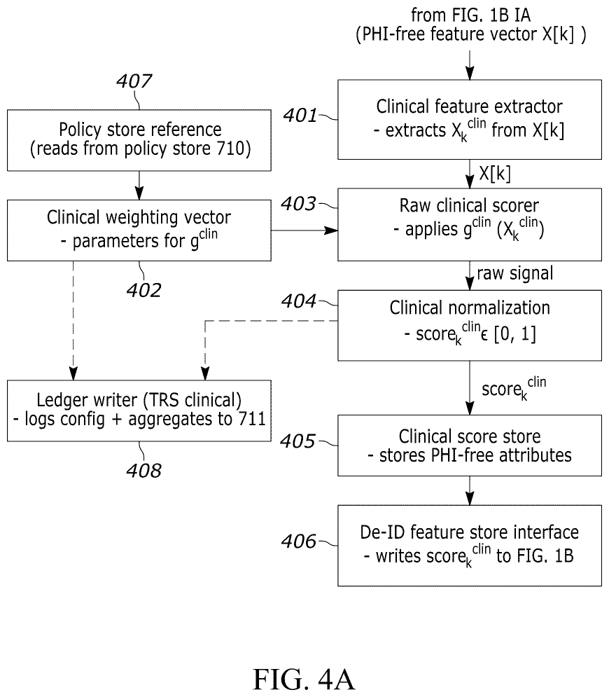

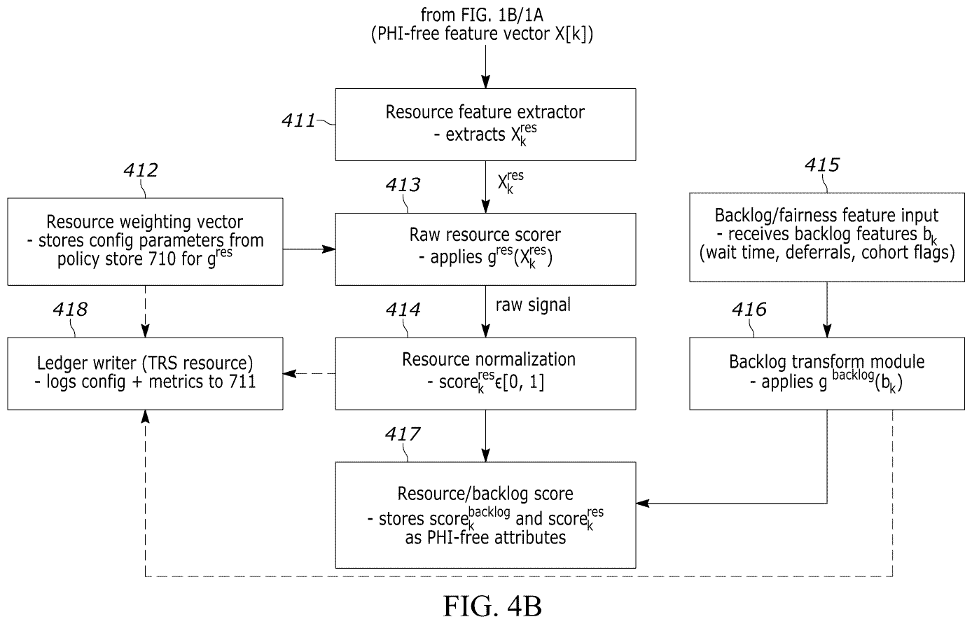

FIG. 1A illustrates a conflict graph encoding mutual restrictions among demand entities in a constrained operational environment, shown in the illustrative context of surgical scheduling. FIG. 1A includes case nodes 101, which represent de-identified demand entities (e.g., surgical cases), each associated with a deterministic linkage identifier (DID) token k∈ [symbol] generated as described with reference to FIG. 1B. In some embodiments, the DID token is generated by a keyed tokenization function such that the resulting function is pseudorandom-appearing, does not reveal underlying direct identifiers, and is computationally infeasible to reverse without access to a facility-controlled secret or mapping. In some embodiments, linkage between the DID token and an underlying identifier is maintained in a secure registry or vault to enable longitudinal correlation across time while preserving de-identification within an optimization compute plane. For example, a DID token may be mapped to an original encounter identifier in a vault accessible only to authorized operations within a controlled re-identification enclave.

Conflict edges (k, k′) ∈E 102 are established between nodes 101 to encode mutual restrictions, such as shared personnel, room/time overlap, precedence constraints, or shared resource requirements. In some embodiments, conflict edges 102 carry weights reflecting a severity, frequency, or policy-defined penalty associated with a conflict, thereby enabling downstream treatment of certain conflicts as soft penalties versus hard exclusions. In other embodiments, conflict edges 102 are unweighted and conflicts are treated as binary exclusions derived from policy rules, credential checks, and availability or readiness signals.

Each case node 101 may be annotated with node attributes 103 derived from a de-identified feature representation X[k]. The node attributes 103 exclude direct identifiers and may include, by way of example, duration estimates, service line or procedure class indicators, risk proxies, resource requirements, financial-efficiency features, and equipment or implant flags. In some embodiments, the node attributes 103 include perioperative readiness indicators and coverage or authorization status encoded as non-identifiable flags. In some embodiments, the node attributes 103 include learned representations or embeddings derived from structured operational events to improve conflict detection, turnover estimation, and duration forecasting.

The de-identified feature vector X[k] 108 for each node 101 is produced by a feature transform operator (e.g., Φ_{θ}) described with reference to FIG. 1B. In some embodiments, the de-identified feature vector X[k] 108 is engineered from canonicalized data feeds using time-grid normalization and feature standardization. In other embodiments, the de-identified feature vector X[k] 108 is produced by one or more learned encoders that map heterogeneous clinical, operational, and administrative inputs into representations suitable for constraint construction, feasibility evaluation, and optimization within an optimization compute plane. In some embodiments, X[k] 108 comprises one or more component feature sets, such as

$${X}_{k}^{clin} , {X}_{k}^{res} ,$$

and optionallly

$${X}_{k}^{fin}$$

which may be combined, weighted, and/or gated for downstream decision control.

A conflict matrix 104 provides an adjacency or matrix representation of a conflict graph G=(V, E), where the matrix is symmetric and diagonal entries are zero (i.e., no self-loops). In some embodiments, the conflict graph is unweighted and represented as G=(V, E). In other embodiments, the conflict graph is weighted and represented as G=(V, E, W), where W assigns a conflict weight to at least a subset of edges (k, k′) ∈E to represent severity, frequency, or a policy-defined penalty class. For example, 104[i, j]=1 indicates a conflict between two demand entities i and j. In such embodiments, a weight W(i, j) is associated with at least a subset of conflicting pairs to enable soft versus hard exclusivity in downstream feasibility and optimization. In some embodiments, the conflict matrix 104 is stored in a sparse format to support efficient solver integration, such as persisting only non-zero entries for large schedules. In some embodiments, the conflict representation is extended to a directed or layered structure to represent precedence, contamination, or infection-control sequences that impose temporal orderings beyond undirected conflicts.

The graph builder/validator 110 is a subsystem that constructs, validates, and maintains the conflict graph from de-identified inputs. For example, the graph builder/validator 110 rejects an edge or constraint that would permit double-booking of a shared resource (e.g., a surgeon) across overlapping room-time assignments. In some embodiments, the graph builder/validator 110 applies schema validators and policy validators to ensure only permitted, de-identified fields and attributes enter a graph tier or optimization compute plane, and performs consistency checks against credentialing, staffing, and equipment registries. For example, the graph builder/validator 110 confirms that a credential, privilege, or specialty permits assignment of a provider to a proposed case. In some embodiments, the graph builder/validator 110 incorporates anomaly detection to flag improbable durations, duplicate entries, or cyclic precedence constraints prior to graph promotion. For example, the graph builder/validator 110 may flag a twelve-hour estimated duration for a procedure that typically completes in ninety minutes.

A graph/coloring partitioner or coloring module 105 assigns labels, colors, or partitions to nodes 101 to produce mutually compatible (e.g., conflict-free) classes of demand entities. For example, the partitioner 105 groups cases into sets that can be scheduled in parallel without violating encoded conflicts. In some embodiments, the partitioner 105 uses graph coloring, clique cover, maximum independent set heuristics, or related combinatorial methods to form compatible groups. In some embodiments, the partitioner 105 uses optimization-based relaxations, greedy approximations, or spectral methods to approximate large compatible sets under encoded constraints and policy limits.

The color-to-slot mapping module 106 maps graph colors or partitions to resource-time pairs (r, t) such as operating rooms r and time slots t∈ [symbol] . For example, a partition labeled “blue” may be mapped to Room 3 at 07:00 and Room 5 at 07:15. In some embodiments, the mapping module 106 is dynamic and reflects block ownership, turnover expectations, staffing windows, and real-time availability signals. In some embodiments, mapping module 106 is policy-driven and aligns partitions to service-line templates, surgeon preferences, or day-of-week capacity profiles. For example, orthopedic partitions may be mapped to Tuesday/Thursday high-capacity templates.

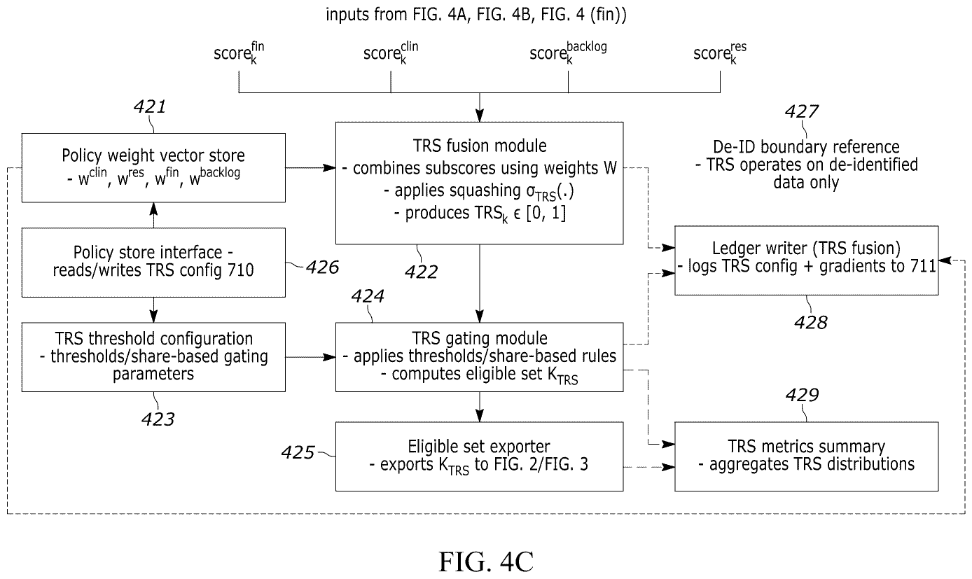

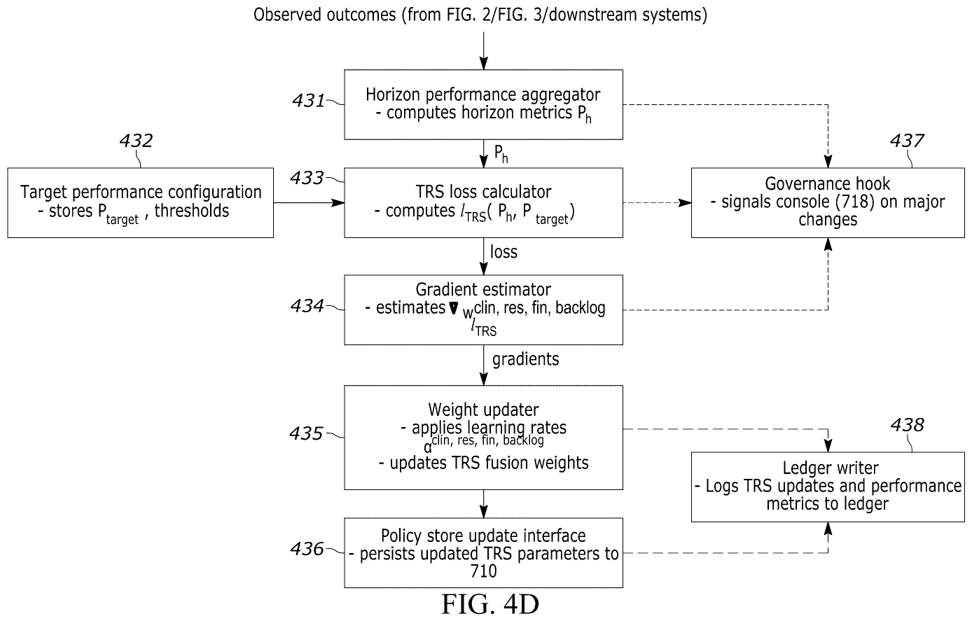

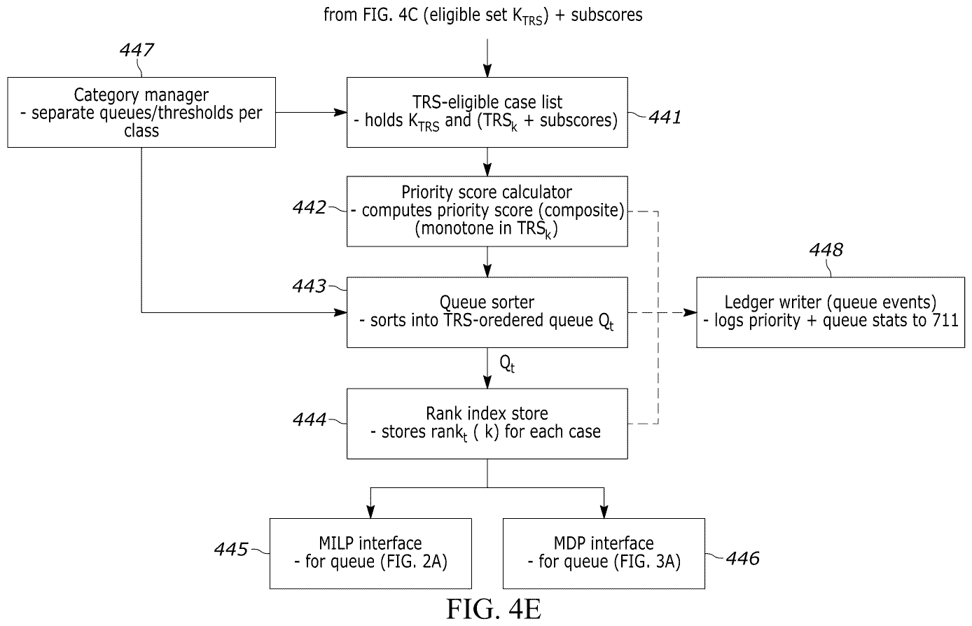

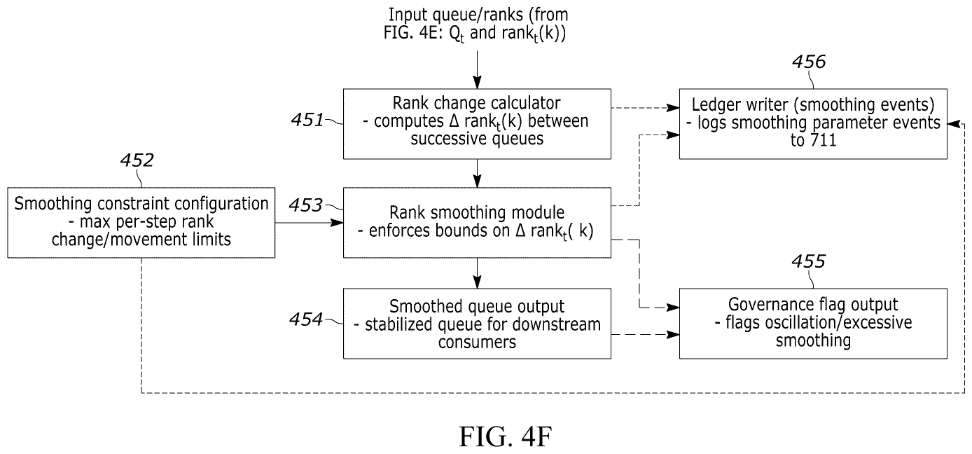

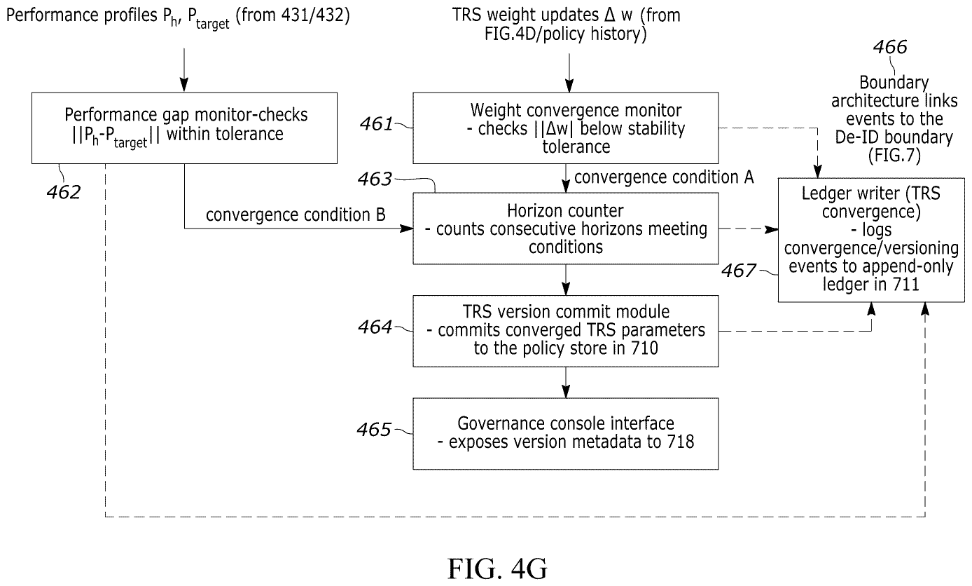

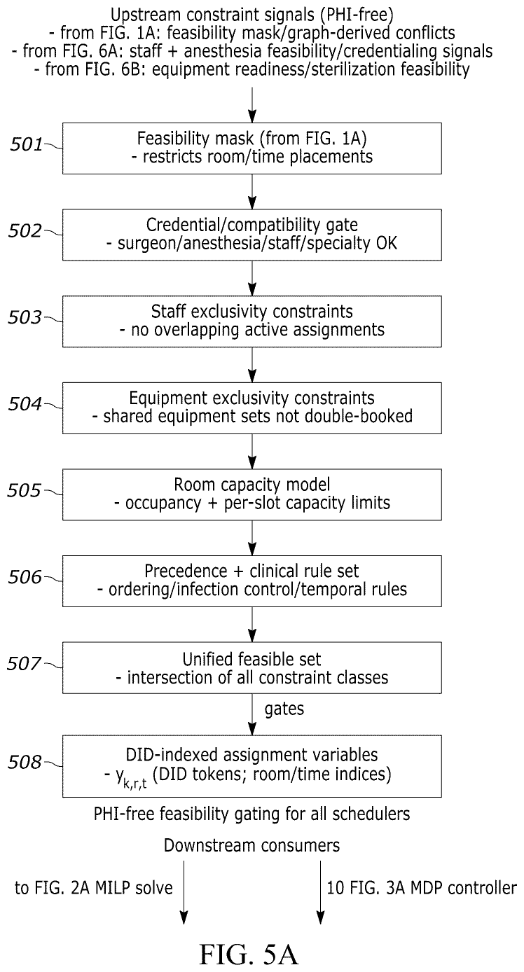

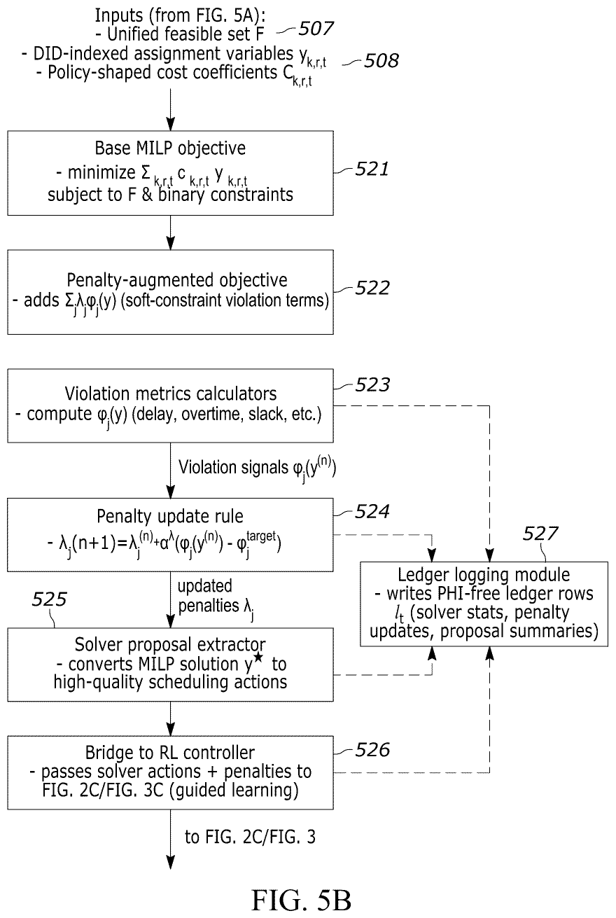

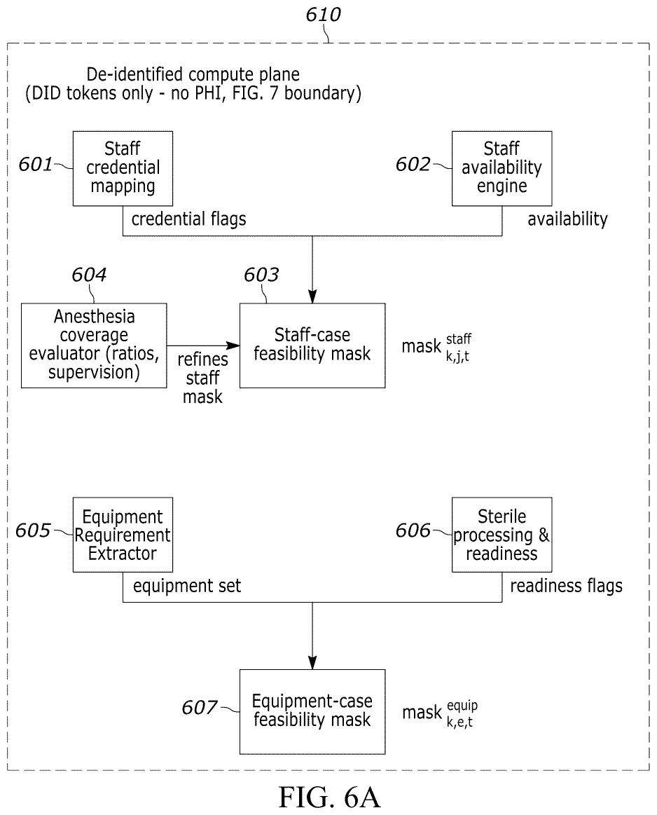

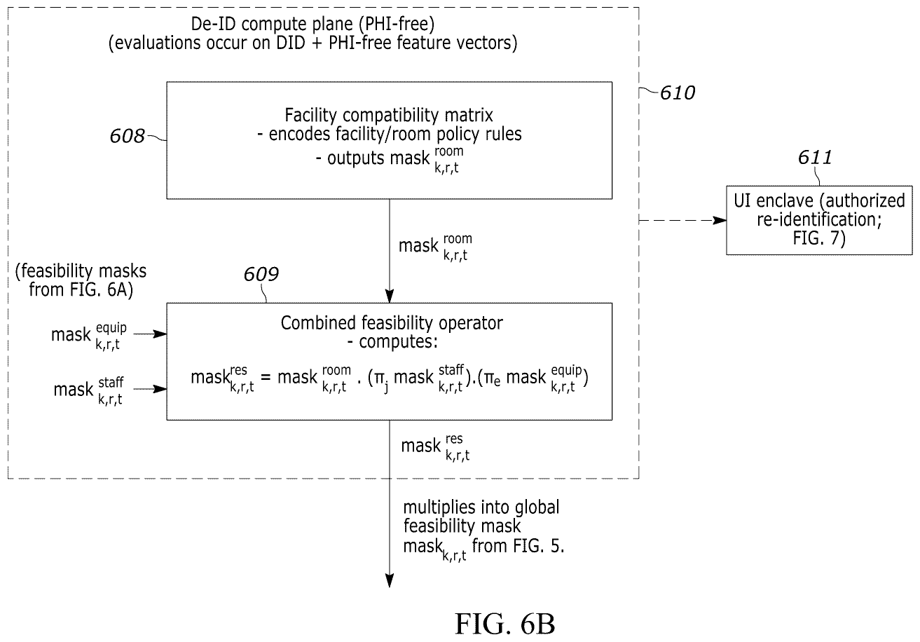

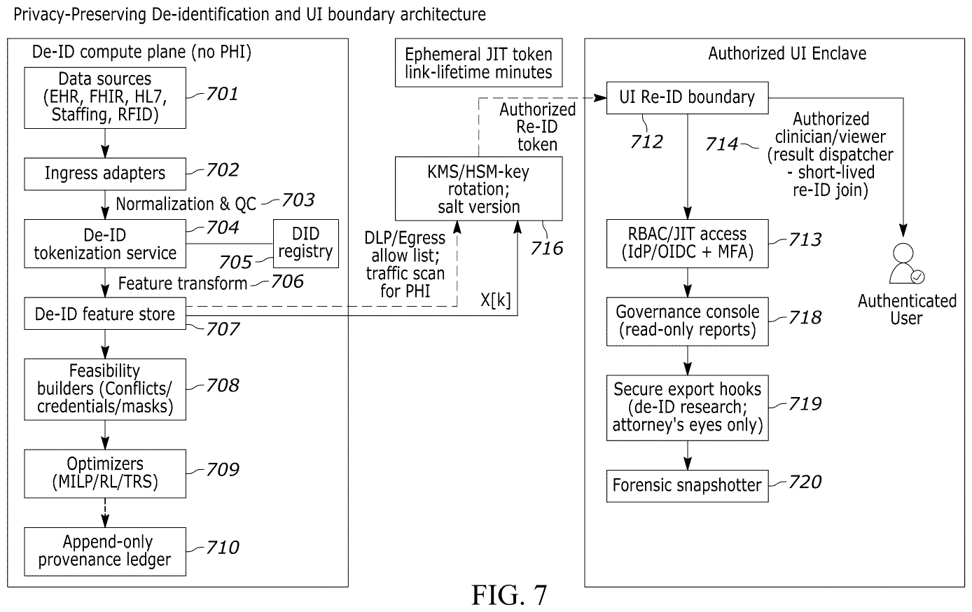

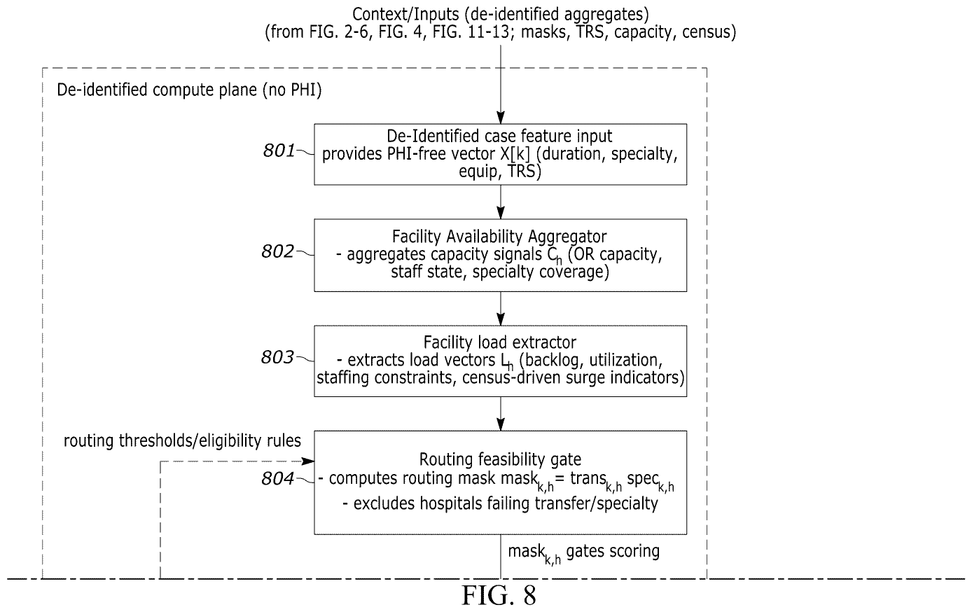

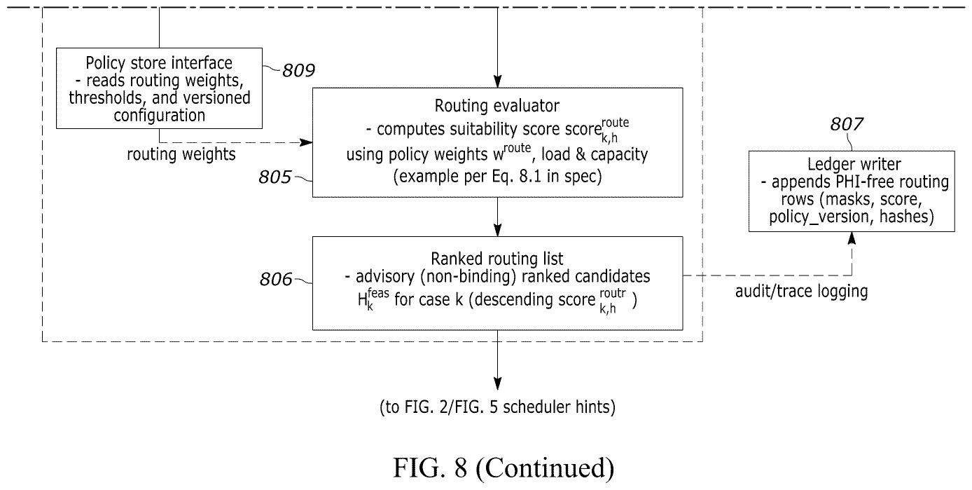

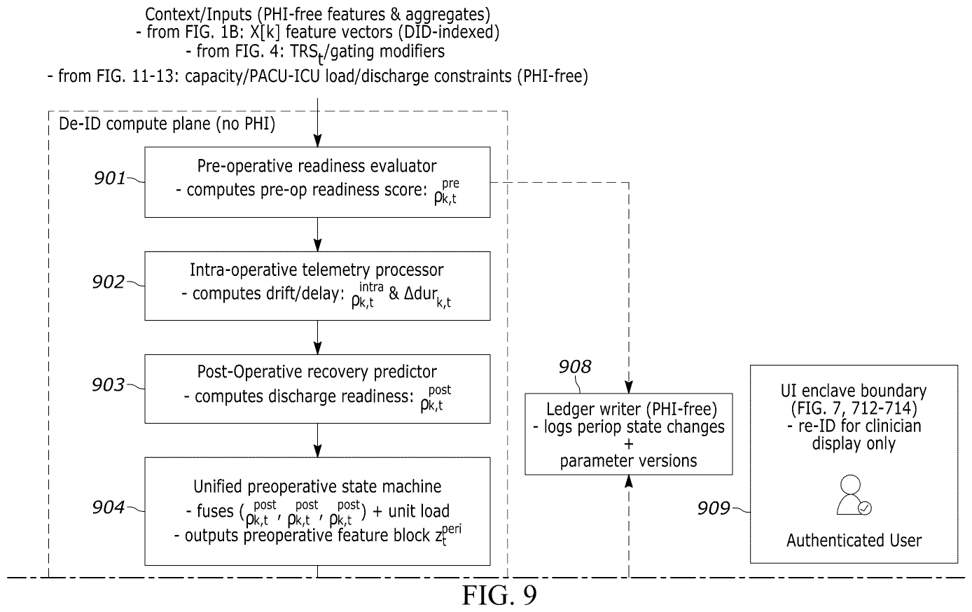

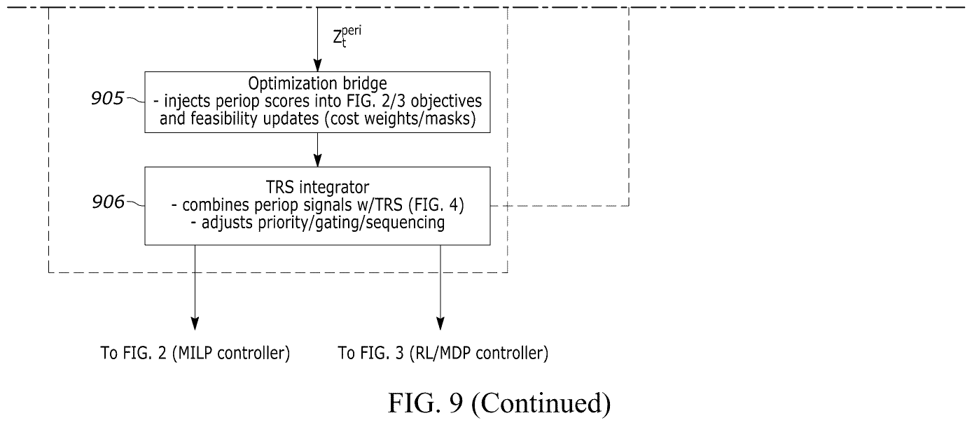

The feasibility mask output 107 comprises one or more derived masks mask_{k,r,t} exported to the mixed-integer linear programming (MILP) and/or reinforcement learning (RL) controllers described with reference to FIG. 1C and FIGS. 2A-2C. The feasibility masks indicate, for each demand entity k, resource r, and time slot t E [symbol] , whether a candidate assignment or action is admissible under encoded constraints. For example, mask_{k,r,t}=1 permits demand entity k to start in resource r at time slot t, and mask_{k,r,t}=0 excludes the assignment. In some embodiments, the feasibility masks 107 incorporate staff credential gates, anesthesia coverage ratios, equipment readiness signals, bed-type compatibility, infection-control constraints, and payer or authorization feasibility encoded as non-identifiable indicators. For example, the feasibility mask output 107 disallows a slot if required trays are not yet sterilized. In some embodiments, feasibility masks 107 are layered and combined such that a base graph-derived admissibility mask is combined with one or more resource-specific feasibility operators to yield a unified admissibility indicator per resource-time slot. In some embodiments, the feasibility masks 107 are generated and consumed within an optimization compute plane that excludes direct identifiers and are keyed by the deterministic linkage identifiers k.

The feasibility mask output 107, together with the conflict graph structures and any derived compatibility classes, may be stored in a graph store/cache 111 for downstream use. For example, the graph store/cache 111 retains the latest mask artifacts for reuse by an assignment engine or solver. In some embodiments, the store/cache 111 versions each artifact with timestamps and policy version identifiers and applies time-to-live rules for stale partitions or masks. For example, the graph/store cache 111 expires masks after a shift change or upon detection of an updated availability signal. In some embodiments, the store/cache 111 exposes a ledger-linked index to support reproducible solver runs and governance review. For example, an index may reference a specific mask set and associated policy version used for a particular scheduling cycle to reproduce a decision record appended to a tamper-evident ledger.

In some embodiments, an identity-separated de-identification and linkage boundary 109 is provided, wherein direct identifiers are confined to a controlled zone, and an optimization compute plane operates on deterministic linkage identifiers k ∈ [symbol] and de-identified feature representations. Within the boundary 109, a DID registry or facility-controlled mapping enables longitudinal correlation across records without exposing direct identifiers to the conflict graph, feasibility, or optimization tiers. In some embodiments, the boundary 109 enforces that re-identification is confined to a controlled re-identification enclave, such as a secure user interface (UI) enclave, with role-based access control, policy enforcement, and egress filtering. For example, patient names or other direct identifiers are presented only within the controlled re-identification enclave to authorized perioperative staff or administration. In some embodiments, the boundary 109 supports mixed-mode deployments in which limited identifiable attributes may be used under stricter access policies, while the conflict graph and feasibility tiers remain de-identified (e.g., excluding direct identifiers) to preserve auditability, portability, and reproducibility.

FIG. 1B illustrates a de-identification and feature extraction pipeline. The pipeline begins with hospital data sources 121, which may include EHR/ADT feeds, anesthesia information management systems (AIMS), OR boards, laboratory and imaging systems, census feeds, credentialing databases, and related clinical and operational sources. These data streams are ingested through typed ingress interfaces 122, such as HL7v2 handlers, FHIR interfaces, batch file parsers, or message-queue adapters, which validate schemas, normalize incoming records, and classify fields according to policy. For example, the interfaces 122 may map heterogeneous HL7 messages into a canonical format and drop, tokenize, or route direct-identifier fields designated for exclusion from an optimization compute plane. In some embodiments, the ingress interfaces 122 tag and route de-identified fields destined for the conflict graph components of FIG. 1A, including case nodes 101, node attributes 103, and the de-identified feature vector X[k] 108.

The aggregator/canonicalizer 123 combines heterogeneous data streams into a canonical record representation for each encounter or demand entity. A temporal alignment engine 124 then aligns events to a discrete time grid [symbol] with slot width Δ, normalizing timestamps across feeds to ensure temporal consistency. For example, disparate time stamps are aligned to fifteen-minute slots. A data quality and imputation module 125 resolves missing fields, sparsity, and anomalies, thereby producing temporally aligned and cleaned records. For example, the module 125 may impute a missing duration estimate using a historical median or model-based estimate. In some embodiments, aligned records from the temporal alignment engine 124 provide inputs for node attributes 103 used in graph construction, and cleaned duration estimates are used to generate or refine the conflict matrix 104.

At this stage, the pipeline may split into two paths. In a protected-field handling and tokenization path, a router 128 diverts direct-identifier fields (e.g., names, medical record numbers (MRNs), and dates of birth) to a DID registry or token vault 129 that maintains a facility-controlled mapping between underlying identifiers and a deterministic linkage identifier k ∈ [symbol] outside an optimization compute plane. In an identity-separated optimization compute plane, a feature transform operator Φ_{θ} 126 converts the aligned records into de-identified feature vectors X[k] 108 that exclude direct identifiers and stores the resulting pairs (k, X[k]) in a de-identified feature store 127. For example, the feature transform operator Φ_{θ} 126 may emit features indicating procedure category, equipment flags, and duration estimates. In some embodiments, the feature vectors X[k] 108 are provided to the graph builder/validator 110 to populate case nodes 101 and to compute conflict edges (k, k′) ∈E 102 based on encoded attributes in 103. For example, a shared personnel attribute may yield a conflict edge (k,k′) ∈E 102.

A policy cache 132 stores active policies, constraints, parameters and weights that govern downstream processing. A mask assembler 133 constructs feasibility masks mask_{k,r,t} and related constraint artifacts for downstream solvers, and a constraint solver interface 134 bridges the de-identified feature store 127 and the feasibility masks to one or more optimization and learning controllers. For example, the mask assembler 133 may combine staff feasibility and equipment readiness constraints into a unified room-time admissibility mask, and the constraint solver interface 134 exposes the resulting masks to a solver. In some embodiments, the mask assembler 133 supplies feasibility mask outputs 107 for FIG. 1A and persists them along with graph artifacts in the graph store/cache 111. In some embodiments, the constraint solver interface 134 references the color-to-slot mapping 106 and the conflict matrix 104 (and, in some embodiments, conflict weights W) to ensure that only mutually compatible assignments are exposed to the solvers.

In some embodiments, operations in the pipeline are recorded in an append-only ingestion/provenance audit ledger 130 as immutable ledger rows [symbol] ₜ, thereby providing a tamper-evident log of ingestion events, feature transformation versions, policy configurations, and, in some embodiments, references to downstream decision outputs recorded in a separate decision ledger. A controlled re-identification enclave 131, such as a secure user interface (UI) boundary, supports authorized re-identification and display, ensuring that linkage to underlying identifiers is confined to the enclave and excluded from the optimization compute plane. For example, a perioperative dashboard may resolve a deterministic linkage identifier k ∈ [symbol] to a patient label only within the controlled re-identification enclave 131. In some embodiments, the controlled re-identification enclave 131 is coordinated with the de-identification and linkage boundary 109 of FIG. 1A such that re-identification associated with case nodes 101 and feasibility masks 107 occurs only within the enclave, while the conflict graph and feasibility tiers operate on deterministic linkage identifiers k ∈ [symbol] and de-identified attributes that exclude direct identifiers. In some embodiments, the audit ledger 130 is cryptographically chained and supports replay and governance review of recorded events and associated policy versions. For example, governance may authorize re-running an optimization under a prior policy configuration for audit or comparison purposes.

FIG. 1C depicts a state-action-reward decision control framework. A controller 135 selects scheduling actions over a feasible set of candidate assignments generated from feasibility masks and constraints. In some embodiments, the controller 135 includes one or more optimization and/or learning modules, such as mixed-integer linear programming (MILP), reinforcement learning (RL), or a hybrid thereof, to evaluate candidate actions over admissible assignments. For example, the controller 135 may use feasibility masks 107 to restrict candidate case-room-time starts to admissible assignments. The controller 135 operates on de-identified feature inputs 136 that exclude direct identifiers, including feature vectors X[k] 108, feasibility masks 107, and constraint artifacts provided by upstream modules such as the mask assembler 133 and the constraint solver interface 134. For example, the de-identified feature inputs 136 may aggregate current masks, objective coefficients, and policy-defined weights for a next solve. In some embodiments, the controller 135 further references conflict structures maintained in the graph store/cache 111 and validation outputs from the graph builder/validator 110 to ensure consistency between feasible actions and encoded conflicts. For example, the controller 135 may verify that a proposed assignment does not violate an updated conflict entry in the conflict matrix 104.

The controller 135 selects one or more assignments or actions for scheduling, ensuring that only assignments allowed by the feasibility masks 107 are considered. Selected actions may be recorded in an append-only decision/optimization ledger 138 that stores auditable decision records, including references to associated inputs (e.g., feature vectors 108 and feasibility masks 107), outputs, and an active policy version identifier (e.g., policy_versionₜ). In some embodiments, entries in the decision ledger 138 are cross-linked to ingestion and transformation records stored in the append-only ingestion/provenance ledger 130 (e.g., immutable ledger rows [symbol] ₜ) to preserve end-to-end provenance from data intake and feature transformation through decision output and presentation.

A versioned policy store 139 maintains a current set of policy parameters, reward weights, tradeoff weights, and governance rules that shape optimization and learning behavior of the controller 135. For example, the versioned policy store 139 may store a configuration that increases weights on downstream capacity feasibility when ICU utilization pressure rises. Updates to policy parameters, such as changes in objective coefficients, constraint priorities, or gating thresholds, may be retrieved from the policy store 139 and revised based on observed outcomes, override events, and audit feedback. In some embodiments, the policy store 139 retains version metadata aligned with feasibility masks 107 and de-identified feature vectors X[k] 108 so that solver behavior can be reproduced against specific graph artifacts, partitions, and input snapshots stored in the graph store/cache 111.

A controlled re-identification enclave 137 serves as a secure interface boundary for presenting scheduling outputs to authorized users. Re-identification of deterministic linkage identifiers k ∈ [symbol] via the DID registry or token vault 129 is permitted only within the controlled re-identification enclave 137 (which, in some embodiments, corresponds to the UI boundary 131 described above), thereby confining exposure of direct identifiers to display, notification, or workflow handoff under role-based access controls and egress rules. For example, an export or print-to-file action performed outside the enclave 137 may include only de-identified summaries. In some embodiments, re-identification events at the enclave 137 are logged for auditability and compliance and linked to related pipeline events recorded in the audit ledger 130. In some embodiments, the controlled re-identification enclave 137 is the same component as the UI boundary in 131.

In some embodiments, the controller 135 operates as a hybrid optimization engine that blends MILP and RL techniques to adaptively refine scheduling policies while preserving feasibility imposed by feasibility masks 107 and conflicts encoded via the conflict matrix 104 and the graph builder/validator 110. For example, the controller 135 may learn to reduce late starts by increasing delay penalties while still enforcing hard feasibility constraints. In some embodiments, the decision ledger 138 is implemented as a tamper-evident log, such as a cryptographically chained ledger (e.g., hash-chained records, a Merkle structure, or a blockchain-backed implementation), and is cross-linked to ingestion and transformation records in the audit ledger 130 for unified governance and reproducibility. For example, alteration of a past assignment entry is detectable via a hash mismatch. In some embodiments, the versioned policy store 139 is dynamically updated in response to observed outcomes and performance metrics Ph derived from decision records and feasible action evaluations (e.g., inputs 136 and feasibility masks 107), and from governance review, thereby supporting continuous improvement of scheduling decisions under the de-identification and linkage boundary 109. For example, a governance process may approve a policy revision when repeated anesthesia supervision violations are detected.

FIG. 2A illustrates a mixed-integer linear programming (MILP) assignment engine. A MILP engine 201 executes computations within an identity-separated optimization compute plane using de-identified feature vectors X[k] 108 that exclude direct identifiers, feasibility masks 107, and constraint artifacts provided via the mask assembler 133 and the constraint solver interface 134. In some embodiments, the engine 201 references conflict structures from the conflict matrix 104 and graph artifacts stored in the graph store/cache 111 to ensure alignment between encoded conflicts and admissible assignments. In some embodiments, the engine 201 retrieves policy-shaped parameters and objective coefficients from the versioned policy store 139 (and, in some embodiments, via a runtime policy cache) to maintain consistency with current governance rules. For example, the engine 201 may use updated delay penalties and weighting parameters from the versioned policy store 139 when constructing an objective function.

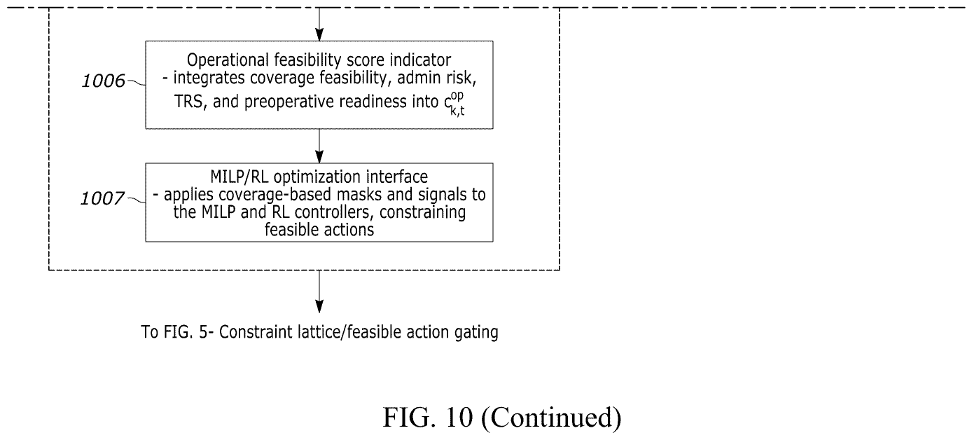

A feasibility matrix module 202 receives room-time feasibility masks 107 and related constraint artifacts derived from the conflict matrix 104 and, in some embodiments, from the unified constraint lattice described with reference to FIG. 5A, thereby conditioning an admissible assignment space on encoded exclusivity, capacity, and timing limits. For example, the module 202 enforces at most one active case per room per time slot. In some embodiments, the module 202 integrates multiple feasibility and eligibility constraints—including credential eligibility indicators (e.g., cred_{k,r}) and time-dependent availability masks assembled from staff, anesthesia, equipment, and downstream capacity signals—to form a combined admissibility indicator for each (k,r,t) assignment. For example, the module 202 may combine a credential eligibility matrix with an equipment readiness mask to produce a unified feasibility mask entry mask_{k,r,t}. In some embodiments, the module 202 updates feasibility snapshots when upstream availability changes are detected by the constraint solver interface 134.

A credential matrices module 203 receives surgeon/room and role-based eligibility constraints and produces one or more credential feasibility indicators, ensuring that only assignments compatible with credentialed personnel and clinical eligibility rules are considered. In some embodiments, the module 203 produces a credential matrix cred_{k,r} indicating whether demand entity k is clinically eligible for assignment in room r (e.g., based on privileges, room capability, service line rules, or specialty requirements), independent of time. In some embodiments, time-dependent staffing constraints—such as shift windows, break rules, supervision ratios, and fatigue limits—are expressed as additional availability constraints or feasibility masks that are combined upstream (e.g., by the feasibility matrix module 202) to determine whether an otherwise credential-eligible assignment is admissible at a particular timeslot. For example, an assignment that is credential-eligible under cred_{k,r} may still be disallowed at a time slot if required personnel are unavailable at that time. In some embodiments, the module 203 or the feasibility matrices module 202 derives a time-indexed credential feasibility indicator cred_{k,r,t} by combining cred_{k,r} with time-dependent availability constraints.

A cost generator 204 computes MILP cost coefficients c_{k,r,t} for candidate assignments, incorporating factors such as throughput, delay propagation, and resource burden, and, in some embodiments, financial efficiency, and parameterizing such coefficients according to policy weights and operational priorities retrieved from the versioned policy store 139. For example, the cost generator 204 penalizes late starts and overtime minutes in an objective function. In some embodiments, the cost generator 204 incorporates capacity feasibility, discharge opportunity, and coverage-readiness signals as additive or multiplicative modifiers to bias selection toward safer and administratively feasible schedules. For example, assignments that would admit patients when ICU capacity is scarce may receive increased cost. In some embodiments, the cost generator 204 applies learned or rule-based normalization so that cost scales remain stable across changing case-mix. For example, the cost generator 204 rescales coefficients when a daily case count or case mix fluctuates.

A solver component 205 performs MILP optimization over assignment variables y_{k,r,t}, subject to feasibility masks and credential constraints provided by modules 202 and 203. The solver component 205 returns an optimal or near-optimal set of assignments that respect encoded constraints. For example, the solver component 205 produces a schedule that improves utilization while avoiding disallowed overlaps and constraint violations. In some embodiments, the solver component 205 supports soft-constraint penalties and adaptive penalty updates, such as dual-based updates or feedback-driven coefficient adjustment, to reduce repeated violations over time while preserving hard feasibility imposed by feasibility masks 107 and encoded conflicts in the conflict matrix 104. For example, repeated overtime events may increase an associated penalty coefficient q over successive optimization cycles according to learning rate aa, thereby discouraging repeated violations. In some embodiments, the solver component 205 produces one or more proposal sets or candidate schedules for the controller 135 to evaluate in a closed-loop policy refinement process.

A ledger writer 206 records each optimization cycle by writing hash-chained rows to an append-only decision/optimization ledger 138, thereby supporting traceability and compliance together with ingestion and transformation entries recorded in the append-only ingestion/provenance audit ledger 130. For example, the ledger writer 206 logs an input-hash for feasibility artifacts and an output-hash for an assignment result. In some embodiments, the ledger writer 206 stores input-hashes summarizing feasibility masks 107, de-identified feature vectors X[k] 108, and a policy version identifier retrieved from the versioned policy store 139, alongside output hashes summarizing selected assignments and solver statistics. For example, the ledger writer 206 records utilization and overtime metrics for an optimization cycle. In some embodiments, the ledger writer 206 cross-links entries to feasibility artifacts persisted in the graph store/cache 111 to enable reproducible analysis. For example, the ledger writer 206 records a pointer to an exact mask set used by the feasibility matrices module 202.

An output dispatch module 207 exports optimized assignments and summary signals to the controller 135 for closed-loop policy refinement and continuous improvement of scheduling decisions. In some embodiments, the module 207 conveys objective costs, constraint dual values or sensitivity indicators, and violation metrics to the controller 135 so that policy parameters and weights in the versioned policy store 139 can be updated under governance review. In some embodiments, the module 207 filters proposed actions against feasibility masks 107 prior to presentation or execution, thereby preventing propagation of infeasible assignments downstream.

In some embodiments, components of the MILP assignment engine 201 operate on PHI-free feature vectors X[k] 108 and DID-indexed artifacts within a de-identification and linkage boundary 109, with re-identification and user-facing presentation confined to a secure user interface (UI) enclave 137. In some embodiments, the engine 201 applies time-grid normalization consistent with the temporal alignment engine upstream so that feasibility masks and assignment variables are evaluated over discrete intervals. In some embodiments, the engine 201 supports policy-version pinning such that a given optimization cycle is tied to a specific policy version in the versioned policy store 139 and can be reproduced using artifacts referenced from the graph store/cache 111 and ledger entries in the audit ledger 130 and the decision ledger 138.

FIG. 2B shows an RL controller that operates over a de-identified scheduling state. A state encoder 221 builds a PHI-free representation of a current scheduling state using de-identified features X[k] 108, feasibility masks 107, and recent assignment outputs from the MILP engine 201. In some embodiments, the state encoder 221 also references conflict structures from the conflict matrix 104, feasibility classes produced by the graph/coloring partitioner 105, and room-time mappings from 106, such that the encoded state remains consistent with an admissible action space. For example, the state encoder 221 masks out actions that would violate partition-based exclusivity. In some embodiments, the state encoder 221 aggregates capacity and availability signals emitted through the constraint solver interface 134 and mask assembler 133 and normalizes timestamps to the discrete time grid [symbol] established upstream. For example, the state encoder 221 appends ICU load indicators to a state vector.

An action set input 222 receives a feasible action set [symbol] ^{feas}(sₜ) defined by feasibility masks 107 and eligibility constraints supplied via the credential matrices module 203 (e.g., cred_{k,r}), ensuring that only admissible scheduling actions are considered. In some embodiments, the action set input 222 further filters actions by additional resource feasibility indicators generated in module 202, including staff-, equipment-, and bed-level admissibility, and prunes actions that would violate exclusivity encoded in the conflict matrix 104. In some embodiments, the action set input 222 applies policy-based throttles retrieved from the versioned policy store 139 to limit exploration when safety or governance conditions are active.

A reward module 223 computes rewards R(sₜ, aₜ) for actions, capturing operational metrics such as throughput, delay propagation, and, in some embodiments, financial efficiency, derived in whole or in part from MILP cost coefficients c_{k,r,t} produced by the cost generator 204. For example, actions that reduce overtime may receive higher reward. In some embodiments, the reward module 223 incorporates capacity feasibility, discharge opportunity, and coverage-readiness signals as modifiers so that rewards reflect downstream bed pressure, administrative risk, and perioperative readiness. In some embodiments, the reward module 223 applies smoothing or scaling functions so that reward magnitudes remain stable across changing case mix and across policy versions recorded in the versioned policy store 139. For example, rewards may be normalized per-day to reduce sensitivity to case count.

The value and/or Q-function approximator 224 estimates long-run returns for state-action pairs, supporting policy evaluation and improvement. For example, a Q-function approximator Q_{θ}(sₜ, aₜ) may estimate an expected utilization gain associated with advancing a case. In some embodiments, the value and/or Q-function approximator 224 is trained using temporal-difference updates based on realized outcomes logged in the append-only decision/optimization ledger 138, with optional cross-reference to ingestion and transformation records in the append-only ingestion/provenance audit ledger 130 to preserve provenance. For example, the approximator 224 updates parameters based on prior-cycle utilization, delay, and cancellation outcomes. In some embodiments, the approximator 224 maintains separate approximators per feasibility class or room category to improve stability under heterogeneous constraints derived from the conflict matrix 104, the graph/coloring partitioner 105, and feasibility masks 107.

A policy parameter output 225 exports updated policy parameters (e.g., 0) and/or cost-weight vectors that are used to adjust the cost generator 204 in the MILP engine 201, and, in some embodiments, to update policy configuration maintained in the versioned policy store 139 under governance review. In some embodiments, the policy parameter output 225 emits penalty coefficients for soft-constraint handling in solver component 205, thereby enabling adaptive penalty updates that reduce repeated violations while preserving hard feasibility imposed by feasibility masks 107. For example, overtime penalty coefficients may increase when repeated overtime is observed. In some embodiments, the policy parameter output 225 tags each export with version metadata aligned with the versioned policy store 139 such that solver behavior can be reproduced using artifacts from the graph store/cache 111 and ledger records in the audit ledger 130 and the decision ledger 138. For example, a policy export includes hashes for feasibility masks 107 and feature inputs X[k] 108.

A ledger snapshot reader 226 imports PHI-free ledger snapshots from the append-only decision/optimization ledger 138, allowing the controller to align training and policy updates with an actual deployed policy history. For example, the ledger snapshot reader 226 may train on schedules recorded over a time prior window. In some embodiments, the ledger snapshot reader 226 filters snapshots by policy_versionₜ and/or feasibility-mask hash to ensure consistency between training data and current constraint sets derived from feasibility masks 107 and eligibility constraints supplied via module 203. For example, the ledger snapshot reader 226 joins snapshot metrics with graph artifacts in the graph store/cache 111 and conflict summaries in the conflict matrix 104 to detect drift in an admissible action space and to trigger conservative exploration or safety modes.

In some embodiments, operations in FIG. 2B are performed within a de-identification and linkage boundary 109 using de-identified feature vectors X[k] 108, feasibility masks 107, and solver outputs from the MILP engine 201, and any re-identification for user-facing presentation occurs only within the controlled UI enclave 137 under role-based access control. In some embodiments, the RL controller of FIG. 2B operates in a hybrid loop with the MILP engine 201 by consuming proposal sets from solver component 205 and returning updated policy parameters or weights via the policy parameter output 225, thereby implementing closed-loop policy refinement consistent with governance rules stored in the versioned policy store 139 and audit requirements enforced by the append-only decision/optimization ledger 138, with ingestion and transformation provenance retained in the append-only ingestion/provenance audit ledger 130.

FIG. 2C shows a hybrid RL and MILP execution loop. A hybrid execution loop 231 integrates MILP assignments and signals from the MILP engine 201 with RL policy outputs from the RL controller comprising modules 221-226. For example, the loop 231 ingests an assignment set from solver component 205 together with updated policy parameters emitted by the policy parameter output 225 to coordinate a next scheduling cycle. In some embodiments, the hybrid execution loop 231 includes a weight update rule 232 and a performance metric aggregator 233 that operate over de-identified inputs X[k] 108, feasibility masks 107, and constraint artifacts including the conflict matrix 104 and graph artifacts stored in the graph store/cache 111. For example, the aggregator 233 can compute utilization and overtime metrics from assignment outputs produced by the solver component 205 (e.g., assignments y_{k,r,t}) and combine such metrics with conflict counts derived from the conflict matrix 104 to produce a daily feasibility-pressure or constraint-violation summary.

The performance metric aggregator 233 collects operational metrics such as utilization, delay, cancellations, and, in some embodiments, financial efficiency and constraint violations from recent scheduling cycles produced by solver component 205 under cost coefficients generated by the cost generator 204. For example, the aggregator 233 computes per-room overtime minutes, same-day cancellation rates, and throughput per hour across a prior number N of cycles. In some embodiments, the aggregator 233 ingests capacity-aware signals derived from the feasibility matrices module 202 and eligibility constraints supplied via the credential matrices module 203 (e.g., cred_{k,r}), and correlates policy version identifiers from the versioned policy store 139 with realized outcomes logged in one or more append-only ledgers, including the ingestion/provenance audit ledger 130 and the decision/optimization ledger 138. In some embodiments the aggregator 233 computes horizon-level summaries, including average overtime per room, TRS-conditioned cancellation rates, downstream bed overflow incidents, and queue stability indices aligned with feasibility classes from the graph/color partitioner 105 and room-time mappings from 106. For example, when high-TRS cases repeatedly coincide with ICU overflow, the aggregator 233 emits a capacity-risk alert associated with a current policy version stored in the versioned policy store 139.

The weight update rule 232 updates policy parameters (e.g., θ) based on aggregated metrics from the performance metric aggregator 233 and rewards R(sₜ, aₜ) computed by the reward module 223. For example, if repeated late starts are observed for a particular block window, the update rule 232 increases delay penalties and reduces exploration for that window. In some embodiments, the update rule 232 applies adaptive updates to soft-constraint penalties used by solver component 205 (e.g., feedback-driven adjustments), increasing penalties for repeated violations while maintaining hard feasibility imposed by feasibility masks 107 and encoded conflicts in the conflict matrix 104. In some embodiments, the update rule 232 applies temporal-difference-style updates that tune cost-weight vectors exported via the policy parameter output 225 such that the cost generator 204 re-weights throughput, delay, financial efficiency, and capacity signals on subsequent solves. In one example embodiment, if the performance metric aggregator 233 detects sustained ICU overflow risk associated with particular start windows, the update rule 232 increases weights on capacity feasibility terms and reduces exploration via throttles configured in the versioned policy store 139, thereby biasing assignments away from the higher-risk windows while preserving an admissible action space defined by feasibility masks 107 and eligibility and availability constraints supplied via modules 202 and 203.

A compliance logging interface 234 records each iteration of the execution loop, including policy updates, performance metrics Pₕ, and optimization outputs, thereby supporting traceability and auditability. In some embodiments, the compliance logging interface 234 appends a compliance record to the append-only decision/optimization ledger 138, where the compliance record includes cryptographic hashes of de-identified inputs X[k] 108, feasibility masks 107, and conflict references derived from the conflict matrix 104; a policy version identifier and/or active policy parameters referenced from the versioned policy store 139; and an output hash summarizing an assignment set produced by solver component 205. In some embodiments, each compliance record written to the decision/optimization ledger 138 includes a pointer to one or more corresponding ingestion and transformation records stored in the append-only ingestion/provenance audit ledger 130, thereby preserving end-to-end provenance from intake through optimization. In some embodiments, the compliance records are hash-chained within the decision/optimization ledger 138 and are exposed only as de-identified summaries to the governance console 235, without re-identification outside the de-identification and linkage boundary 109.

A governance console 235 provides a user interface for authorized human oversight, enabling approvals, holds, and controlled revisions of policy versions. For example, the governance console 235 presents a comparison of utilization and cancellation metrics before and after a weight update rule 232 and allows an administrator to approve or reject a proposed change. In some embodiments, the governance console 235 surfaces versioned policy information from the versioned policy store 139, feasibility summaries from modules 202 and 203, conflict statistics derived from the conflict matrix 104 and graph artifacts stored in the graph store/cache 111, and training snapshots from the ledger snapshot reader 226 to authorized stakeholders for transparent oversight. In some embodiments, the governance console 235 displays side-by-side comparisons of recent policy updates and their effects on utilization, delay, and violation metrics to support approval workflows. In one example embodiment, if governance users review compliance records indicating repeated fallback schedules due to anesthesia supervision violations the governance console 235 authorizes a policy revision or temporarily restricts exploration via throttles maintained in the versioned policy store 139.

In some embodiments, the hybrid execution loop 231 enables closed-loop feedback between MILP and RL components, supporting ongoing enhancement of scheduling policies under governance and compliance controls. In such embodiments, computations are performed on PHI-free feature vectors X[k] 108, feasibility masks 107, and DID-indexed artifacts within a de-identification and linkage boundary 109, and any re-identification occurs only within a controlled user interface enclave 137. For example, the hybrid execution loop 231 operates on a discrete time grid [symbol] consistent with temporal alignment performed by module 124 such that performance metrics Pₕ, rewards R(sₜ, aₜ), and updated cost weights apply per interval. In other embodiments, the hybrid execution loop 231 aggregates across variable horizons to accommodate different planning windows while maintaining consistency with feasibility masks 107 and constraints derived from the conflict matrix 104 and feasibility and eligibility modules 202 and 203.

FIG. 3A shows a de-identified scheduling control architecture that operates on top of the feasibility and optimization engines described previously. A state encoder 301 builds a PHI-free scheduling state from de-identified features X[k] 108 and recent assignment decisions produced by solver component 205, aggregating information such as current queues, schedules, throughput risk scores, and census signals (e.g., census_{u,t}). For example, the state encoder 301 encodes room occupancy and schedule progress from solver outputs (e.g., assignments y_{k,r,t}), a remaining case queue derived from case nodes 101, and capacity indicators computed from bed- and unit-level feasibility signals. In some embodiments, the state encoder 301 references conflict structures derived from the conflict matrix 104, feasibility partitions from the graph/coloring partitioner 105, and room-time mappings from 106 to ensure that an encoded state aligns with an admissible action space. In some embodiments, the state encoder 301 normalizes timestamps to a discrete time grid [symbol] produced by the temporal alignment engine 124.

A feasible-action set builder 302 constructs an admissible set of scheduling actions by applying feasibility masks 107 and associated eligibility and availability constraints supplied via modules 202 and 203, thereby ensuring that only actions consistent with encoded resource, staffing, and policy requirements are considered. For example, the feasible action set builder 302 filters out actions that would double-book a surgeon or assign a case to a room when required equipment is not ready (e.g., sterilization readiness signals). In some embodiments, the feasible action set builder 302 further applies coverage feasibility constraints (e.g., described with reference to FIG. 10) and bed/census feasibility constraints (e.g., described with reference to FIG. 11-12) to restrict actions when downstream capacity is insufficient.

A transition estimator 303 predicts an impact of candidate actions on a system state, including changes in room occupancy, backlog, and downstream capacity. For example, the transition estimator 303 projects updated room occupancy using solver outputs (e.g., assignments y_{k,r,t}) and duration and turnover estimates, updates backlog counts and propagates capacity effects into a length-of-stay (LOS)-aware census forecast (e.g., as described with reference to FIG. 12). In some embodiments, the transition estimator 303 uses learned dynamics based on historical trajectories recorded in one or more append-only ledgers, including ingestion/provenance records in audit ledger 130 and decision/optimization records in decision ledger 138. In some embodiments, the transition estimator 303 applies rule-based turnover, duration drift, and precedence rules derived from conflict structures (e.g., conflict matrix 104) and constraint artifacts (e.g., described with reference to FIG. 5A).

A reward shaper 304 computes a reward signal for each action, incorporating operational metrics such as throughput, delay propagation, and, in some embodiments, financial efficiency, weighted according to active policy parameters maintained in the versioned policy store 139. For example, the reward shaper 304 increases rewards R(sₜ, aₜ) for actions that reduce overtime and avoid downstream ICU overdflow while honoring feasibility masks 107 and associated eligibility and availability constraints. In some embodiments, the reward shaper 304 incorporates discharge opportunity scores (e.g., as described with reference to FIG. 13) and capacity feasibility indicators (e.g., as described with reference to FIGS. 11-12) as modifiers. In some embodiments, the reward shaper 304 applies smoothing or scaling such that reward magnitudes remain stable across variable case mix and across policy versions.

A value/Q-evaluator 305 estimates a long-term expected value of feasible actions using a dynamic-programming, Bellman evaluation, or temporal-difference evaluation module, thereby supporting policy evaluation and improvement. For example, the value/Q-evaluator 305 estimates an expected utilization improvement associated with advancing a high-TRS case earlier while accounting for predicted downstream bed pressure (e.g., as described with reference to FIGS. 11-13). In some embodiments, the value/Q-evaluator 305 maintains separate evaluators per feasibility partition (e.g., as described with reference to FIG. 1A) or per room category to improve stability under heterogeneous constraints derived from conflict structures (e.g., conflict matrix 104) and feasibility masks 107.

An action selector 306 selects an action at from a feasible action set based on value estimates. In one example embodiment, the action selector 306 selects

$${a}_{t}^{★} = arg {max}_{a ϵ {𝒜}^{feas} ( {s}_{t} )} {Q}_{θ} ( {s}_{t} , a ) ,$$

where [symbol] ^{feas} (sₜ) is constrained by feasibility masks 107 and associated eligibility and availability constraints (e.g., as described with reference to FIGS. 1A-2B). For example, the action selector 306 selects an action that maximizes an estimated value subject to feasibility masks 107 and credential eligibility constraints supplied via module 203. The selected action is provided to a MILP interface 307 that maps the selected action into an optimization instance solved by the MILP engine (e.g., as described with reference to FIG. 2A), including converting the selected action into decision variables and constraints for solver component 205 while maintaining hard feasibility.

A policy store interface 308 reads and writes policy parameters θ and policy-version information stored in the versioned policy store 139 to support traceable policy updates. For example, the policy store interface 308 retrieves current delay weights prior to action evaluation and stores updated weights following learning epochs.

A ledger writer 309 records each decision, including a representation of the state, a selected action, value estimates, and a policy version identifier, in an append-only decision ledger (e.g., decision ledger 138) to support compliance and governance. For example, the ledger writer 309 appends a hash-chained record linking the decision to input feasibility artifacts (e.g., masks 107), feature vectors X[k] 108, and, in some embodiments, a pointer to related ingestion and transformation records recorded in an append-only ingestion/provenance audit ledger (e.g., audit ledger 130).

FIG. 3B shows a learning and convergence monitoring layer that operates within the de-identified scheduling control system. A de-identified outcome stream 311 ingests PHI-free outcomes from prior scheduling cycles, including schedules, delays, overruns, utilization metrics, throughput risk scores, and discharge opportunity scores (e.g., as described with reference to FIG. 13). For example, the de-identified outcome stream 311 collects daily overtime minutes per room, cancellation rates stratified by TRS tiers, and ICU overflow incidents. A value/Q update engine 312 applies mask-aware updates to a value function and to policy parameters θ using ingested outcomes, thereby supporting policy evaluation and improvement while ensuring that reinforced or promoted actions remain within an admissible action space defined by feasibility masks 107 and associated constraints. For example, the value/Q update engine 312 updates value estimates associated with actions that consistently reduce late starts while respecting feasibility masks 107. In some embodiments, the learning and convergence monitoring layer computes convergence indicators (e.g., moving-average reward, policy-drift magnitude, or constraint-violation rate) to trigger governance review (as described with reference to FIG. 15).

A convergence monitor 313 tracks learning-stability metrics, including value-function changes, parameter drift, policy drift, and, in some embodiments, Bellman residuals, and/or temporal-difference error statistics, to assess stability and convergence of a learning process. For example, the convergence monitor 313 flags when a magnitude of parameter updates remains below a threshold across N epochs, indicating stable convergence. A learning tracker 314 records learning epochs, monitored metrics, and experiment configurations, thereby providing a traceable record of taraining and evaluation. For example, the learning tracker 314 stores a training horizon, reward-shaping parameters (e.g., as described with reference to FIG. 3A), and hashes or identifiers of feasibility artifacts (e.g., feasibility masks 107) used in each epoch.

A versioned audit ledger writer 315 writes PHI-free learning events and policy versions to an append-only decision/optimization ledger (e.g., decision ledger 138) to support compliance, traceability, and reproducibility (e.g., as described with reference to FIGS. 1B-2C). For example, the versioned audit ledger writer 315 appends a record including updated policy weights, snapshot references to the de-identified outcome stream 311, and a pointer to related ingestion and transformation records recorded in an append-only ingestion/provenance audit ledger (e.g., audit ledger 130). A convergence visualization module 316 provides graphical views of learning and convergence metrics for authorized user review, and a UI boundary reference 317 ensures that any visualization that incorporates contextual identity information is displayed only within a controlled re-identification enclave (e.g., UI enclave 137 as described with reference to FIG. 1C). For example, the convergence visualization module 316 renders a drift plot and a value-convergence chart, with the UI boundary reference 317 enforcing that any patient-context overlays are restricted to the controlled re-identification enclave 137. In some embodiments, modules 311-317 operate under a de-identification and linkage boundary (e.g., boundary 109 as described with reference to FIG. 1A) and produce only PHI-free outputs outside the controlled re-identification enclave. In some embodiments, the convergence visualization module 316 provides governance-facing summaries without exposing direct identifiers (e.g., aggregate drift statistics by room, block, or service line).

FIG. 3C illustrates a policy version commit process within a de-identified scheduling control and learning layer. The process begins with evaluating one or more stability thresholds εᵥₐₗᵤₑ, εₚₐᵣₐₘ, ε_{policy}, and, in some embodiments, ε_{TD} and/or ε_{BR} (component 321) against convergence and stability metrics, such as including value differences, parameter drift, policy drift, and, in some embodiments, temporal-difference error statistics and/or Bellman-residual metrics (e.g., as described with reference to FIG. 3B). For example, the threshold evaluator 321 may require a rolling window of low error statistics and bounded parameter drift before considering a commit. When the configurable criteria are satisfied, a policy version commit module 322 commits a new policy version identifier to the versioned policy store 139. For example, the policy version commit module 322 writes a new version identifier (e.g., “v6.1”) to the versioned policy store 139 together with associated metadata including hashes and/or pointers to stored artifacts corresponding to feasibility masks 107 and conflict artifacts 104. A governance console 323 displays version identifiers, timestamps, and performance metrics Pₕ to authorized users, thereby enabling human oversight of policy changes. For example, the governance console 323 displays before/after utilization, delay, and constraint-violation metrics for a proposed policy version. A compliance hook 324 links each policy version commit event to a compliance and boundary architecture by ensuring that commit events are recorded in an append-only decision/optimization ledger (e.g., decision ledger 138) and remain traceable to related ingestion and transformation records (e.g., as recorded in audit ledger 130). For example, the compliance hook 324 writes a chain-linked record containing commit-criteria results, policy metadata, and one or more pointers to related audit-ledger entries.

In some embodiments across FIG. 3A-3C, the control, learning, and governance layers operate on PHI-free feature vectors X[k] 108, feasibility masks 107, conflict artifacts derived from the conflict matrix 104, and assignment outputs produced by the MILP engine (e.g., as described with reference to FIGS. 2A-2C) within a de-identification and linkage boundary (e.g., boundary 109). Any re-identification required for display, workflow handoff, or authorized override occurs only within a controlled re-identification enclave e.g., UI enclave 137) under role-based access control. For example, the action selector 306 and policy version commit module 322 may execute exclusively on de-identified artifacts, while the governance console 323 presents re-identified context only within the controlled re-identification enclave 137 for authorized review and approval.

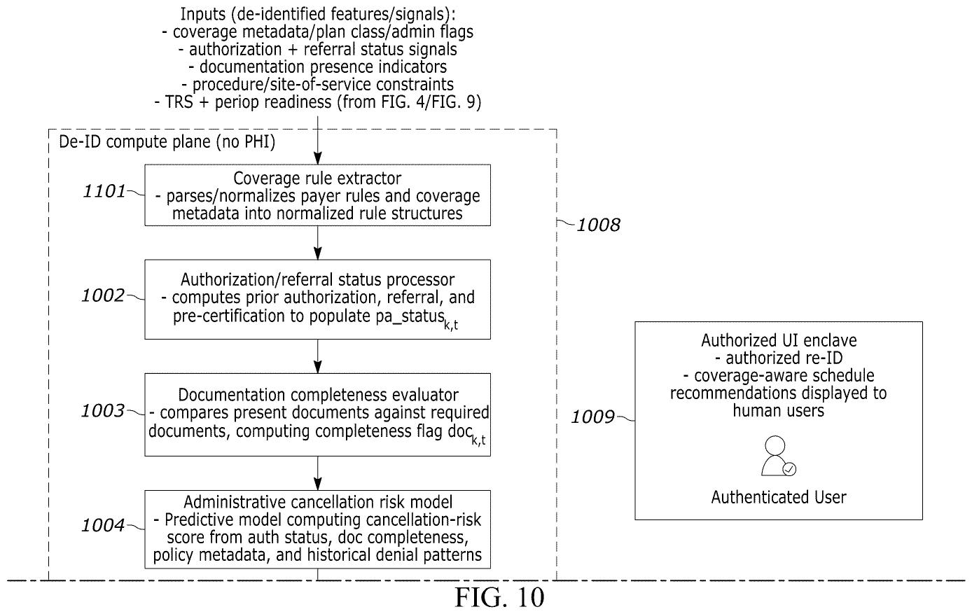

FIG. 4A shows a clinical urgency scoring process that operates on de-identified features X[k] 108 and deterministic linkage identifiers k ∈ [symbol] . A clinical feature extractor 401 receives a PHI-free feature vector X[k] 108 and extracts a clinical subvector

$${X}_{k}^{clin}$$

relevant to urgency assessment. For example, extractor 401 selects normalized ASA class, comorbidity flags, and ICU propensity indicators encoded in X[k]. A clinical weighting vector 402 provides configuration for a clinical scoring function g^{clin}(·), and, in some embodiments, parameters of vector 402 are read via a policy store reference 407 from the versioned policy store 139. For example, vector 402 increases weight on ICU propensity when the versioned policy store 139 indicates elevated downstream risk. A raw clinical scorer 403 applies the configured scoring function to the extracted clinical features to generate an intermediate clinical signal, for example,

$${g}^{clin} ( {X}_{k}^{clin} ) .$$

A clinical normalization module 404 then normalizes the intermediate signal to produce a bounded clinical score

$${score}_{k}^{clin}$$

∈[0, 1]. The resulting clinical score is stored as a de-identified attribute in a clinical score store 405 and is provided via a de-identified feature store interface 406 for downstream use by scheduling and control components (e.g., as described with reference to the MILP engine 201 of FIG. 2A and the RL controller modules 221-226 of FIG. 2B). A ledger writer 408 records the scoring configuration and summary statistics to an append-only ingestion/provenance audit ledger (e.g., audit ledger 130) for compliance and traceability. For example, ledger writer 408 records a policy-version identifier from the versioned policy store 139 and a distribution summary of

$${score}_{k}^{clin}$$

by service line. In some embodiments, when the clinical score is used to influence a scheduling decision, the audit entry is cross-linked to a corresponding decision record in an append-only decision ledger (e.g., decision ledger 138 as described with reference to FIG. 1C and FIG. 2A) to preserve end-to-end provenance.

FIG. 4B shows a resource and backlog scoring process that operates on de-identified features X[k] 108 and deterministic linkage identifiers k ∈ [symbol] (as described with reference to FIGS. 1A-1B). A resource feature extractor 411 receives a PHI-free feature vector X[k] 108 and extracts a resource/operational sub vector

$${X}_{k}^{res} .$$

For example, extractor 411 selects duration forecasts, turnover sensitivity, equipment complexity flags, and staffing burden indicators encoded in X[k] 108 (e.g., as described with reference to the feature transform operator Φ_{θ} 126 of FIG. 1B). A resource weighting vector 412 provides configuration parameters for a resource scoring function g^{res}(·), and a raw resource scorer 413 computes an intermediate resource signal, for example

$${g}^{res} ( {X}_{k}^{res} ) .$$

A resource normalization module 414 normalizes the intermediate signal to produce a bounded resource score

$${score}_{k}^{res}$$

∈ [0, 1]. In parallel, a backlog/fairness feature input 415 receives backlog-related features bₖ for the demand entity, such as wait time, deferrals, or cohort flags derived from queue telemetry (e.g., derived from case nodes 101 of FIG. 1A and state summaries produced by the state encoder 301 of FIG. 3A). A backlog transform module 416 applies a mapping g^{backlog}(bₖ) to yield a normalized backlog score

$${score}_{k}^{backlog}$$

∈[0, 1]. The resulting resource and backlog scores are stored as de-identified attributes in a resource/backlog score store 417 for downstream use by scheduling and control components (e.g., as described with reference to cost generator 204 of FIG. 2A and reward module 223 of FIG. 2B). A ledger writer 418 records the scoring configuration, transformation references, and summary score statistics append-only ingestion/provenance audit ledger (e.g., audit ledger 130) to support compliance and governance review. For example, ledger writer 418 records rolling trends in

$${score}_{k}^{backlog}$$

by specialty and a policy-version identifier to support governed tuning. In some embodiments, when the resource and/or backlog scores are used to shape an optimization objective or to gate admissible actions, the audit ledger entry is cross-linked to a corresponding decision record in an append-only decision ledger (e.g., decision ledger 138 as described with reference to FIGS. 1C and 2A) to preserve traceability from scoring inputs to executed scheduling outputs.

FIG. 4C shows a workflow for fusing and gating Throughput Risk Scores (TRS) using only de-identified features X[k] and deterministic linkage identifiers k ∈ [symbol] . As described with reference to FIGS. 4A-4B, normalized sub scores may be produced for each demand entity k, including a clinical-impact subscore

$${score}_{k}^{c l i n} ,$$

a resource-burden subscore

$${score}_{k}^{res} ,$$

an optional financial subscore

$${score}_{k}^{fin} ,$$

and a backlog/fairness subscore,

$${score}_{k}^{backlog} .$$

A policy weight vector store 421 maintains configurable fusion weights w^{clin}, w^{res}, w^{fin}, w^{backlog} governing the relative contribution of each subscore. A TRS fusion module 422 receives the normalized subscores and combines them according to the stored weights, for example by computing a weighted sum and applying a squashing function σ_{TRS} (·) to produce a composite score TRSₖ bounded to a range such as [0, 1].

A TRS threshold configuration 423 provides one or more eligibility parameters defining which demand entities are eligible for prioritization, including absolute thresholds (e.g., TRSₖ≤τ_{TRS}) and/or share-based gating (e.g., selecting a top fraction of demand entities by TRS). For example, configuration 423 may select the top 30% of demand entities by TRS for add-on consideration. A TRS gating module 424 applies the eligibility parameters to filter the computed TRSₖ values and generate an eligible set [symbol] _{TRS}. The eligible set exporter 425 provides the eligible set to downstream scheduling and optimization engines, for example to the MILP engine of FIG. 2A and the RL modules of FIG. 2B. A policy store interface 426 supports reading and writing of TRS weights and thresholds to the versioned policy store 139, thereby enabling governed updates based on policy changes or observed outcomes. A de-identification boundary reference 427 enforces that TRS fusion and gating operations are performed on de-identified artifacts within a de-identification and linkage boundary (e.g., boundary 109 as described with reference to FIGS. 1A-1C and FIG. 7).

To support auditability and governance, a ledger writer 428 records TRS configuration metadata (including weight and threshold versions) and summaries of the eligible set to one or more append-only ledgers, including (i) an ingestion/provenance audit ledger (e.g., audit ledger 130) for configuration provenance and (ii) a decision ledger (e.g., decision ledger 138) when TRS gating affects an executed optimization or scheduling decision. A TRS metrics summary 429 aggregates distributions of TRSₖ values and related metrics for monitoring and governance review, for example quartiles of TRS by service line and correlation with cancellation rates.

FIG. 4D shows a feedback-driven TRS weight-tuning process that operates on de-identified features X[k] and deterministic linkage identifiers k ∈ [symbol] . A horizon performance aggregator 431 computes horizon-level performance metrics P_{horizon} from de-identified outcomes recorded in one or more append-only ledgers, including (i) configuration and provenance records recorded in the ingestion/provenance audit ledger 130 and (ii) decision and optimization outcomes recorded in the decision/optimization ledger 138. For example, 431 summarizes late starts and ICU overflow incidents over a two-week window. A target performance configuration 432 stores target performance profiles P_{target} and threshold parameters from these metrics. A TRS loss calculator 433 computes a loss value [symbol] _{TRS} (P_{horizon}, P_{target}) by comparing the aggregated performance metrics Pₕ to the configured targets. A gradient estimator 434 estimates ∇_{w}_{^{clin}} [symbol] _{TRS}, ∇_{w}_{^{res}} [symbol] _{TRS}, ∇_{w}_{^{fin}} [symbol] _{TRS}, ∇_{w}_{^{backlog}} [symbol] _{TRS}, indicating the direction and magnitude of weight adjustments needed to reduce the loss/A weight updater 435 applies learning rates α^{clin}, α^{res}, α^{fin}, α^{backlog} to the computed gradients and updates the TRS fusion weights accordingly, for example such as throughput, delays, cancellations, overtime, and downstream capacity events. For example,

$${w}_{new}^{clin} = {w}_{old}^{clin} - {α}^{c l i n} {∇}_{{w}^{c l i n}} {ℒ}_{T R S} .$$

The updated TRS parameters are persisted to the versioned policy store 139 by a policy store update interface 436, and a governance hook 437 signals the governance console 235 when significant TRS changes occur, enabling oversight and review by authorized stakeholders. A ledger writer 438 logs all TRS update events, associated performance metrics Pₕ, and configuration changes to the ingestion/provenance audit ledger 130, supporting compliance and traceability of policy evolution. For example, 438 records that clinical-weight component increased by 10% due to elevated overflow risk, together with associated before/after metrics and a policy version identifier.

FIG. 4E shows a queue management and priority workflow for cases that have been determined to be eligible based on their TRS, operating entirely on de-identified features X[k] 108 and DID tokens k ∈ [symbol] . A TRS-eligible case set [symbol] _{TRS} 441 holds the set of cases and their associated TRS and subscore components that have passed the TRS gating criteria 424. For example, 441 lists cases with TRS≥0.7 along with clinical and backlog components. A priority score calculator 442 computes a composite priority score for each case, typically as a monotone function of the TRS value and, optionally, operational modifiers such as deadlines or fairness adjustments derived from bₖ (e.g., 415). The queue sorter 443 sorts the cases into a TRS-ordered queue Qₜ based on the computed priority scores, producing an ordered list in which higher-priority cases are positioned earlier in the queue. The rank index store 444 records the queue rank rankₜ (k) for each case, enabling downstream systems to reference the current priority ordering. A category manager 447 supports the management of separate queues and thresholds for different case categories, such as surgical specialties or urgency classes, allowing for differentiated prioritization logic across service lines. The prioritized queue is then provided to downstream optimization engines via a MILP interface 445 to MILP engine 201 and an MDP interface 446 to RL controllers 221-226, enabling both mixed-integer linear programming and reinforcement learning controllers to consume the queue for scheduling and decision-making. A ledger writer 448 records queue outputs and priority-ranking artifacts to an append only decision/optimization ledger 138 when such queue outputs are used to influence or generate a scheduling decision. For example, 448 records rank changes after new laboratory results update clinical sub scores.

FIG. 4F shows a workflow for stabilizing queue rankings within the TRS priority workflow layer, operating entirely on de-identified features X[k] 108 and DID tokens k ∈ [symbol] . A rank change calculator 451 determines changes in rank rankₜ (k) for each case between successive queue updates. For example, 451 detects that Case A moved from rank 10 to rank 4 after a TRS increase. A smoothing constraint configuration 452 defines a maximum allowable change in rank per update, and, in some embodiments, cumulative movement limits over a defined time window. For example, 452 may specify that no case can move more than five positions in a single update, or that total movement over several cycles remains within a prescribed threshold. A rank smoothing module 452 enforces these constraints, adjusting the queue so that rank changes remain within configured bounds. If a case attempts to move beyond the allowed limit, 453 restricts the movement to the maximum permitted by 452. This results in a smoothed queue output 454 that provides a stabilized and predictable queue for downstream consumers such as scheduling to optimization engines 201 and 221-226. A governance flag output 455 monitors smoothing activity for excessive oscillation or over-smoothing and, upon detecting such conditions, generates alerts for review by authorized stakeholders. A ledger writer 456 records smoothing events, applied parameters, and any governance flags to the ingestion/provenance audit ledger 130, and, when the smoothed queue influences scheduling decisions, cross-links the audit-ledger entry to a corresponding decision record in the decision/optimization ledger 138. For example, 456 records per-step rank-change bounds and identifies cases whose movement was capped.

FIG. 4G shows a TRS convergence and governance process that operates entirely on de-identified features X[k] 108 and DID tokens k ∈ [symbol] . A weight convergence monitor 461 tracks changes in the TRS fusion weight vector over time and checks whether the magnitude of recent updates remains below a configured stability tolerance. For example, 461 flags convergence when weekly weight deltas are below 1%. A performance gap monitor 462 evaluates whether a discrepancy between observed operational metrics and target performance profiles P_{target} stored in 432 is within a configured performance tolerance. A horizon counter 463 counts consecutive horizons meeting both convergence and performance-gap conditions. When stability and performance criteria are satisfied for a sufficient number of horizons, a TRS version commit module 464 commits converged TRS parameters to the versioned policy store 139. A governance console interface 465 exposes TRS version metadata, performance summaries, and convergence history to authorized stakeholders for review and approval. For example, 465 displays a proposed TRS configuration with before/after cancellation and overflow metrics. A boundary architecture 466 ensures that all TRS convergence and governance operations are performed within the de-identification and linkage boundary 109, restricting any re-identification to the secure user interface enclave 137. A ledger writer 467 records convergence and versioning events to the ingestion/provenance audit ledger 130, and, in some embodiments, cross-links such records to decision/optimization ledger entries 138 for time periods in which the committed TRS version governed executed decisions. For example, 467 appends a commit row referencing a version ID, weight-vector hash, and linked training snapshots from outcome stream 311.

In some embodiments, TRS components 401-467 operate on PHI-free feature vectors X[k] 108, and, in some embodiments, reference feasibility masks 107 and policy versions stored in the versioned policy store 139 (e.g., policy_versionₜ). Outputs of the TRS layer are consumed by feasibility and optimization engines 201 and 221-226, and configuration provenance and tuning events are recorded to the ingestion/provenance audit ledger 130, with executed decision provenance recorded to the decision/optimization ledger 138. Artifacts and summaries may be persisted in caches such as graph store/cache 111, and any re-identification is limited to UI enclave 137 under role-based access control.

FIG. 5A shows a unified constraint lattice for surgical and procedural scheduling. A feasibility mask 501 is generated based on upstream constraint signals, including graph-derived conflicts from conflict matrix 104, staff and anesthesia feasibility from 603/604, and equipment readiness from 607. For example, 501 disallows starting a case in Room 2 at 08:00 if a shared robot 607 is not yet released from sterile processing. The feasibility mask 501 restricts assignments y_{k,r,t} to structurally feasible room r and time placements.

A credential and compatibility gate 502 enforces that only assignments with appropriate surgeon, anesthesia, staff, and specialty compatibility are permitted. For example, 502 blocks assignment of a provider to a case outside privileges. Staff-specific constraints 503 ensure that no staff member is assigned to overlapping active cases. For example, 503 prevents the same anesthesiologist from supervising more simultaneous rooms than permitted. Equipment-specific constraints 504 prevent shared equipment sets from being double-booked across rooms or time slots. For example, 504 disallows two robotic procedures at the same time when only one robot is available.

A room capacity model 505 enforces occupancy constraints such as Σₖ OCC_{k,r,t} (y)≤cap_{r,t} and per-slot capacity limits for each room. For example, 505 ensures that at most one active case occupies a room at a given time slot. A precedence and clinical rule set 506 encodes required surgical ordering, infection-control rules, and other temporal or clinical constraints. For example, 506 enforces “biopsy before resection,” and blocks back-to-back cases requiring extended airborne isolation without sufficient turnover time.

These constraint classes are combined to form a unified feasible set 507, representing the intersection of all constraints [symbol] =∩ₘ [symbol] ^{(m)} so that only assignments satisfying constraints derived from 501-506 are permitted. For example, an assignment is feasible only if it simultaneously passes credential gate 502, staff exclusivity 503, equipment exclusivity 504, room capacity 505, and precedence rules 506.

DID-indexed assignment variables 508 are defined for each de-identified case, room, and time slot y_{k,r,t}, ensuring that constraint enforcement occurs within a de-identified compute plane bounded by boundary 109. For example, y_{k,r,t} is a binary variable indicating a start for DID k in room r at slot t, evaluated under PHI-free feasibility artifacts including mask 501. This unified constraint lattice provides PHI-free feasibility gating for schedulers and downstream optimization engines 201 and 221-226. In some embodiments, unified feasible set 507 is realized as a layered admissibility mask that combines graph feasibility 501 with resource and credential feasibility artifacts (e.g., from 202/203) to yield a single admissibility indicator per case-room-time. In some embodiments, room capacity model 505 incorporates variable capacity per slot to reflect planned turnovers or maintenance windows.

FIG. 5B is a diagram of a relaxation and penalty feedback engine layered on top of the constraint lattice of FIG. 5A. The main component is a base MILP objective

$${\sum }_{k , r , t} {c}_{k , r , t} {y}_{k , r , t}$$

521, which minimizes policy-shaped cost coefficients over the unified feasible set 507 and binary assignment variables 508. For example, 521 penalizes delay and overtime while rewarding on-time starts and balanced utilization. A penalty-augmented objective

$${\sum }_{q} {λ}_{q} {viol}_{q} ( y )$$

522 extends the base objective by adding weighted violation terms for selected soft constraints, such as delay, overtime, or equipment overuse. For example, 522 adds a cost for minutes beyond block limits or clustering too many long cases late in the day.

Violation metrics calculators 523 compute measures of constraint violations viol_{q} (y), including delay over target, staff overtime, equipment overuse, block slack, or turnover drift, based on the de-identified assignment variables 508. For example, 523 computes total overtime minutes per room and counts of equipment conflicts avoided or incurred. A penalty update rule

$${λ}_{q}^{( n + 1 )} = max \{ 0 , {λ}_{q}^{( n )} + {α}^{q} ( {viol}_{q} ( {y}^{( n )} ) - {viol}_{q}^{target} ) \}$$

524 adjusts penalty coefficients using a class-specific learning rate and target for each violation metric, increasing the cost of repeatedly violated constraints and relaxing overly strict ones over time. For example, 524 increases the overtime penalty when overtime persists across days and reduces it when schedules return to within targets.

A solver proposal extractor 525 converts optimal or near-optimal MILP assignments into high-quality scheduling actions, which are passed through a bridge to the RL controller 526 to guide Q-learning and policy convergence in downstream modules 221-226. For example, 525 exports a feasible re-sequencing set that RL uses to update exploration versus exploitation weights. A ledger logging module 527 records solver statistics, penalty updates, and proposal summaries as PHI-free ledger rows in 138, maintaining a tamper-evident history of constraint and penalty behavior inside the de-identification and linkage boundary compute plane bounded by 109. For example, 527 writes hashes for masks 501, decisions 508, violation metrics 523, and updated penalty coefficients from 524 to enable reproducible analysis and governance review in console 235.

In some embodiments, 522 applies category-specific penalties that differentiate late starts, missed pre-op readiness signals from 301/901, and coverage-risk signals from 1006. In some embodiments, 524 uses dual-ascent-style updates coupled to horizon metrics aggregated by 233 in FIG. 2C so penalty weights respond to sustained patterns, such as repeated ICU overflow indicated by 1107/1207. In some embodiments, 525 filters proposals against current feasibility masks 107 and credential gates 203 to ensure all actions forwarded to RL remain admissible under 507.

FIGS. 6A-6B are diagrams of a resource feasibility layer that generates PHI-free feasibility artifacts used to gate scheduling actions prior to optimization or learning. In various embodiments, the resource feasibility layer unifies staff credentialing, staff availability, anesthesia supervision rules, equipment readiness, and facility capability constraints into one or more feasibility masks that are keyed by deterministic linkage identifiers k ∈ [symbol] and evaluated within the de-identification and linkage boundary 109. The resulting feasibility artifacts interoperate with graph-derived conflicts 104, feasibility masks 107, DID-indexed assignment variables y_{k,r,t} 508, and solver components 201-205 to restrict an admissible action space to assignments that are feasible under current resource and policy conditions.

FIG. 6A illustrates the generation of staff and equipment feasibility masks within a de-identified compute plane 610. A staff credential mapping module 601 generates credential feasibility indicators cred_{k,j} that specify whether a staff member j is qualified, privileged, or otherwise permitted to participate in demand entity k represented by case node 101 and de-identified feature vector X[k] 108. For example, 601 reads specialty, privileging, and role tables and outputs cred_{k,j} ∈{0,1} for a specialty or subspecialty surgeon, and/or anesthesia provider whose privileges cover the procedure class encoded in X[k] 108. In some embodiments, module 601 cross-references compatibility gate 502 and precedence rules 506 so credential indicators are suppressed when clinical policy forbids a pairing; in other embodiments, module 601 attaches a non-identifying reason code that can be logged for governance review when a credential denial blocks an otherwise feasible assignment 508.

A staff availability engine 602 computes time-indexed availability signals avail_{j,t} based on shift schedules, break rules, fatigue limits, coverage ratios and facility-level constraints encoded by facility policies (e.g., via module 608 and/or policy parameters maintained in the versioned policy store 139). For example, module 602 outputs an availability vector that excludes time intervals beyond a nurse's scheduled end time and inserts mandated break windows for anesthesia teams. In some embodiments, module 602 incorporates horizon-level adjustments derived from performance metrics aggregated by 233 (FIG. 2C), such as overtime caps approved under governance; in other embodiments, module 602 incorporates temporary diversion or surge windows derived from census forecasts (e.g., 1207) to reflect redeployments.

Credential indicators cred_{k,j} and availability signals avail_{j,t} are combined to form a staff-case feasibility mask

$${mask}_{k , j , t}^{staff}$$

603 that determines whether staff member j is feasible for case k at time slot t on the scheduling grid produced by temporal alignment engine 124. For example,

$${mask}_{k , j , t}^{staff} = 1$$

indicates staff member j is both credentialed and available to participate in case k at slot t, and

$${mask}_{k , j , t}^{staff} = 0$$

excludes the pairing. An anesthesia coverage evaluator 604 refines

$${mask}_{k , j , t}^{staff}$$

by applying supervision ratios, role-mix constraints, and specialty-specific rules consistent with clinical policies and constraint lattice elements 502-506. For example, module 604 enforces attending-to-CRNA ratio caps and blocks pairings that violate cardiac anesthesia requirements. In some embodiments, module 604 emits per-slot supervision counters or stress indicators to the RL controller 221 for exploration throttling; in other embodiments, module 604 writes PHI-free ratio warnings or exceptions to decision ledger 138 to support governed tuning via 232/235.

The equipment feasibility process begins with the equipment requirement extractor 605 that identifies equipment requirements εₖ for each case k from non-identifying flags in X[k] 108 and node attributes 103. For example, module 605 extracts a “robot required” flag and a list of sterile tray identifiers for laparoscopic procedures. A sterile processing and readiness engine 606 tracks sterilization cycles, maintenance status, location, and readiness flags sterile_ok_{e,t} for equipment e, and emits time-indexed readiness signals aligned to the time grid of 124. For example, module 606 marks a camera head as ready only after completion of a sterilization cycle and satisfaction of release criteria. These signals are used to construct an equipment-case feasibility mask

$${mask}_{k , e , t}^{equip}$$

607 that determines whether required equipment is available and ready at time slot t, subject to shared-equipment exclusivity consistent with constraints 504. For example,

$${mask}_{k , e , t}^{equip} = 1$$

only when equipment e ∈εₖ is ready and not double-booked at slot t. In some embodiments, module 607 incorporates travel buffers, turnover buffers, or staging constraints for shared equipment across rooms; in other embodiments, module 607 incorporates maintenance-window exceptions encoded in capacity model 505.

FIG. 6B illustrates integration of staff and equipment feasibility artifacts into a combined resource feasibility representation consistent with the unified constraint lattice 507. A facility compatibility matrix 608 encodes room- and site-level policies, capabilities, and constraints, and produces a room feasibility mask mask_{k,r,t} over case k, room r, and slot t. For example, module 608 blocks assignment of a case to an OR lacking required capabilities (e.g., laminar flow, implant support, imaging) and enforces site-of-service policies such as inpatient versus ASC eligibility. In some embodiments, module 608 incorporates isolation and cohorting compatibility signals (e.g., derived from 1205) to restrict rooms for infection-control precautions; in other embodiments, module 608 tags rooms with block ownership and service-line templates to steer assignments under active policy versions maintained in the versioned policy store 139. In some embodiments, the outputs of modules 603/604 (staff feasibility) and 607 (equipment feasibility) are provided to a resource feasibility combiner (e.g., combined feasibility operator 609) that compiles these feasibility artifacts into one or more case-room-time admissibility masks consumed by feasibility and optimization engines (e.g., 201, 221-226).

The combined feasibility operator 609 integrates the room feasibility mask mask_{k,r,t} from 608 with staff-case feasibility mask

$${mask}_{k , j , t}^{staff}$$

603 and the equipment-case feasibility mask

$${mask}_{k , e , t}^{equip}$$

607 to compute a unified resource feasibility indicator for each case, room, and time. For example, 609

$${mask}_{k , r , t}^{res} = 1$$

only if the room is compatible, at least one valid staff configuration is credentialed and available, and all required equipment items e ∈εₖ are sterilized and available for the full duration implied by X[k] 108 and room capacity model 505. In one example embodiment, a combined resource feasibility mask is computed as:

$${mask}_{k , r , t}^{r e s} = {mask}_{k , r , t} \cdot ( {\prod }_{j ∈ {𝒥}_{k}} {mask}_{k , j , t}^{staff} ) \cdot ( {\prod }_{e ∈ {ε}_{k}} {mask}_{k , e , t}^{equip} ) ,$$

Where [symbol] ₖ denotes required staff roles and/or stafing slots for case k, and εₖ denotes required equipment items or sets dor case k. Each mask term is binary, and the product implements hard feasibility gating such that

$${mask}_{k , r , t}^{res} = 0$$

excludes assignment y_{k,r,t} from the feasible set 507 and from an admissible action space provided to engines 201 and 221-226. In some embodiments, operator 609 applies post-multipliers or additional gating based on downstream capacity feasibility (e.g., 1106) to prevent unsafe occupancy patterns; in other embodiments, operator 609 emits both a hard (binary) admissibility indicator and one or more graded scarcity indicators for use by cost generator 204 or reward shaping (e.g., 223) while solver component 205 preserves hard feasibility.

All evaluations occur within the de-identified compute plane 610, where only deterministic linkage identifiers k∈ [symbol] and PHI-free feature vectors X[k] 108 are used. For example, compute plane 610 computes 603/607/609 exclusively on de-identified artifacts and writes provenance to one or more append-only ledgers (e.g., audit ledger 130 and/or decision ledger 138), including input hashes for feasibility masks and policy version identifiers from the versioned policy store 139. A user interface (UI) enclave 611 provides authorized re-identification for displaying schedules and assignments to clinical users, ensuring compliance with de-identification and linkage boundary 109. For example, UI enclave 611 renders an OR board with patient labels resolved from DID tokens for authorized staff and blocks egress of identifiable data via DLP controls. In some embodiments, 611 surfaces non-identifying reason codes from 601/604/607 for human override and logs override events via ledger writer components (e.g., 309/456); in other embodiments, UI enclave 611 supports de-identified exports via secure export hooks (e.g., 719).

In some embodiments, staff feasibility mask 603 is extended to encode team-level feasibility for multi-room anesthesia supervision and scrub-circulator rotations, enabling modules 202/203 to treat teams as composite resources. In some embodiments, equipment feasibility mask 607 integrates RFID dwell-time analytics to estimate relay times for equipment across rooms and adjusts feasibility accordingly. In some embodiments, combined feasibility operator 609 outputs shadow prices or scarcity indicators reflecting a marginal value of constrained resources (for example, robotic availability), which modules 207 and 223 consume to update exploration throttles and reward shaping. In one example, when module 606 signals a late sterilization release for a robot, module 607 flips readiness at 11:00, module 609 unlocks a 12:00 slot, and modules 201/205 re-solve to advance a high-TRS case earlier while feasibility masks 107 and constraint lattice 507 ensure no double-booking occurs.

FIG. 7 illustrates a two-plane architecture separating de-identified compute from authorized, ephemeral re-identification. The architecture is organized into a de-identified compute plane, where all data processing and optimization occur without the presence of protected health information (PHI), and an authorized user interface (UI) enclave, where re-identification is permitted only under strict access controls and is logged for governance.

Within the de-identified compute plane, data sources 701 such as EHR, FHIR, HL7, staffing, sterile processing, RFID asset tracking, and census feeds are ingested through typed ingress adapters 702, which validate schemas and normalize incoming records. For example, ingress adapters 702 convert heterogeneous HL7v2 messages into a canonical format and drop direct identifiers slated for tokenization. A normalization and quality control module 703 ensures data consistency and alignment. For example, 703 deduplicates repeated ADT events, resolves conflicting timestamps, and snaps event times to the discrete grid used by temporal alignment 124. A de-identification tokenization service 704 generates deterministic de-identified identifiers k∈ [symbol] (DID tokens) for each record, and a DID registry 705 securely stores the minimal linkage needed between real identifiers and DID tokens, isolating PHI from the compute environment. For example, module 704 issues the same DID for an encounter under a given key version Kᵥₑᵣ, and module 705 restricts access to linkage keys to enclave-only operations.

A feature transform module 706 converts normalized records into PHI-free feature vectors X[k] 108, which are stored in the de-identified feature store 707. In one example embodiment, the feature transform is defined as:

$$X [ k ] = {Φ}_{θ} ( {raw}_{k} ) ,$$

where rawₖ denotes identifier-minimized, normalized operational records associated with deterministic linkage identifier k ∈ [symbol] , and Φ_{θ} is a parameterized mapping that excludes direct identifiers and yields a PHI-free representation. For example, module 706 emits vectors containing procedure category, duration on the 124 grid, equipment flags, and ICU propensity used downstream by 401/411. Feasibility builders 708 generate constraints and feasibility masks, such as conflicts from the conflict matrix 104, credentials 603/604, room capacity 505, and equipment readiness 607, which are then consumed by optimizers 709, including mixed-integer linear programming (MILP) 201, reinforcement learning (RL) 221-226, and throughput risk scoring engines 422/424. For example, feasibility builders 708 combine a room feasibility mask mask_{k,r,t} 608 with staff masks

$${mask}_{k , j , t}^{staff}$$

603 and equipment masks

$${mask}_{k , e , t}^{equip}$$

607 to produce combined resource feasibility

$${mask}_{k , r , t}^{r e s}$$

609 that gates engines 201 and 221. All operations and decisions are recorded in an append-only provenance ledger 710, providing a tamper-evident audit trail consistent with ledgers 130 and 138. For example, 710 stores input hashes over 108/107, versioned policy 139, and output hashes summarizing assignments from 205 so that a day schedule can be reproduced under governance console 235.

A cryptographic management system and hardware security module 716 manages rotation of cryptographic elements and salt versions for tokenization and enforces data loss prevention (DLP) and egress allow-list policies to prevent patient health information (PHI) leakage. For example, module 716 rotates HMAC keys for tokenization service 704 on a scheduled cadence and blocks outbound payloads containing identifiers other than DIDs. Ephemeral just-in-time token links are issued for authorized re-identification events with limited lifetimes to minimize risk. For example, an OR coordinator receives a short-lived re-ID token to view labels for a particular schedule within UI enclave 137; and the token expires immediately after use.

The authorized UI enclave is the only environment where re-identification is permitted. The UI re-ID boundary 712 serves as the secure entry point for re-identification, and access is controlled by role-based access control and just-in-time authorization 713, leveraging identity providers and multi-factor authentication. For example, a charge nurse authenticates via 713 before boundary 712 resolves selected DIDs to patient labels for the current board. Authorized clinicians and viewers in module 714 receive short-lived re-identified results for display. For example, module 714 renders patient initials and room assignments, while suppressing full identifiers unless policy permits. The governance console 718 provides read-only access to reports and audit logs for compliance oversight. For example, console 718 shows linked rows from audit and decision ledgers 130/138/710 with policy “v5.2” active, the feasibility masks 107 used, and resulting utilization metrics. Secure export hooks 719 enable export of de-identified data for research or legal review under strict controls. For example, export hooks 719 produces an attorneys-eyes-only dataset containing only 108/107 and assignment summaries without re-identification. A forensic snap shotter 720 captures immutable system snapshots for reproducibility and forensic analysis. For example, forensic snap shotter 720 bundles exact model hashes from 201/224, mask hashes from 107, and a stored policy version from versioned policy store 139 so a schedule can be reconstructed months later.

This architecture ensures that all optimization, learning, and decision-support logic (for example, 201, 221-226, 231-235, 401-467, 501-527, 601-611) operate on de-identified data within de-identification linkage boundary 109, with re-identification strictly confined to the UI enclave 137/712-714 under controlled, auditable conditions. In some embodiments, the enclave enforces outbound DLP 716 so that only PHI-free aggregates (for example, utilization and delay statistics) may leave 137/712. In some embodiments, modules 701-709 integrate event-bus semantics consistent with 717, so updates to masks 107 or conflicts 104 trigger re-solves in engine 201 and policy feedback in loop 231/232. In one example, a late sterile-processing release recorded by module 606 flows through feasibility builders 708 to update feasibility masks 107, optimizers 709 trigger MILP engine 201 to re-solve, and hybrid-loop 231 adjusts weights 232 to reduce late-day overtime while governance console 235 logs the change in ledgers 710/138.

FIG. 8 illustrates a multi-facility routing engine workflow designed to evaluate cross-facility feasibility, load, and capacity using de-identified case features X[k] 108 to produce an advisory ranked list of candidate hospitals. The workflow operates entirely within a de-identified compute plane 610 under de-identification linkage boundary 109, ensuring privacy, compliance, and traceability and interoperating with feasibility masks 107, conflict artifacts 104, policy versions from the versioned policy store 139 (e.g., policy_versionₜ), and ledgers 130/138/710.

The workflow begins with the de-identified case feature input 801, which provides PHI-free feature vectors X[k] 108 for each case k∈ [symbol] . These vectors include, for example, case duration aligned to discrete time grid produced by module 124, clinical specialty, equipment requirements (e.g., as determined by 605), and a throughput risk score (TRS) derived from modules 422/424. For example, a case requiring robotic surgery would include a feature vector indicating a robotic requirement that is later evaluated by equipment feasibility module 607 and may include a high TRS reflecting urgency.

A facility availability aggregator 802 collects capacity signals Ch for each facility h ∈ [symbol] , including room/OR availability signals derived from room capacity model 505, staff readiness signals (e.g., masks 603/604), and site capability and specialty coverage signals derived from facility compatibility matrix 608. For instance, a facility with three available operating rooms and staffed orthopedic coverage yields a capacity vector Ch reflecting available room-time capacity and feasible staffing coverage for orthopedic cases. In some embodiments, module 802 ingests staffed downstream capacity and isolation/cohorting constraints from census engines 1203/1206/1207 and bed feasibility 1106 to reflect downstream unit readiness as part of Cₕ.

A facility load extractor 803 computes load vectors Lₕ that capture operational pressure metrics such as backlog, utilization, staffing constraints, and census-driven surge indicators. For example, a facility experiencing high utilization due to emergency cases yields a load vector Lₕ reflecting elevated occupancy ratios from 1203 and increased overtime indicators computed from horizon metrics aggregator 233 and/or PHI-free ledgered outcomes recorded in decision and provenance ledgers (e.g., 138/710). In some embodiments, module 803 incorporates policy-defined load windows stored in the versioned policy store 139 to differentiate elective blocks from emergent throughput and to compute Lₕ consistently under an active policy version identifier.

A routing feasibility gate 804 evaluates transfer feasibility and specialty compatibility for each case and facility computing a routing feasibility mask mask_{k,h}, for example:

$${mask}_{k , h} = {trans}_{k , h} \cdot {spec}_{k , h} ,$$

where trans_{k,h} encodes transfer feasibility and spec_{k,h} encodes specialty/site compatibility. Facilities that fail transfer or specialty requirements are excluded from further consideration. For example, a facility lacking required robotic capability is excluded by spec_{k,h}=0, and a facility lacking appropriate isolation capability per 1205 is masked out for airborne-precaution cases. In some embodiments, module 804 integrates payer and site-of-service constraints (e.g., 1001/1006) to exclude facilities that cannot host the procedure under coverage policies, thereby setting mask_{k,h}=0 for non-permitted placements.

The routing evaluator 805 calculates suitability scores

$${score}_{k , h}^{route}$$

for facilities will mask_{k,h}=1, using policy-defined weights w^{route} to balance load relief and available capacity. For example, a facility with low operational load from 803 and high slack capacity from 802 receives a higher

$${score}_{k , h}^{route}$$

for a case requiring near-term scheduling. In some embodiments, 805 biases

$${score}_{k , h}^{route}$$

using TRS from 422/424 and capacity feasibility indicators (e.g., 1106/1107) so higher-risk cases prefer facilities with stronger downstream bed availability; in other embodiments, module 805 applies geographic or network preference terms read via policy store interface 809.

A ranked routing list module 806 generates an advisory, non-binding ranked list of candidate facilities based on the computed suitability scores

$${score}_{k , h}^{route} .$$

Facilities are ordered in descending suitability, with the top-ranked facility being the most appropriate candidate for the case under the active routing policy. For example, module 806 lists Facility A first when A exhibits lower load and higher staffed ICU capacity compared to Facilities B and C. In some embodiments, module 806 returns multiple top candidates together with uncertainty or confidence indicators so downstream schedulers (e.g., MILP engine 201 and RL controller 221-226) can evaluate alternatives under feasibility masks 107.

A ledger writer 807 appends routing evaluations, including routing masks from 804, suitability scores from 805, and policy-version metadata from the versioned policy store 139, as PHI-free hash-chained rows to an append-only ledger (e.g., decision ledger 138 and/or provenance ledger 710). This logging supports traceability and auditability of routing recommendations. For example, ledger writer 807 records the facility score vector, the active routing weights w^{route}, and hashes of feasibility masks used by 804 so routing advice can be reproduced and reviewed in governance console 235. In some embodiments, ledger writer 807 cross-links routing rows to upstream ingestion and transformation records recorded in audit ledger 130 to preserve end-to-end provenance from data ingest through routing output.

A policy store interface 809 provides routing weights, thresholds, and versioned configurations to guide routing evaluator 805 and routing feasibility gate 804. For example, interface 809 may adjust weights to prioritize facilities within specific geographic regions, to favor facilities with overflow relief needs signaled by 1207, or to enforce temporary transfer freezes during surge periods. In some embodiments, interface 809 pins routing runs to a policy version identifier and tags exported routing artifacts accordingly so reruns can be reproduced using cached artifacts from graph store/cache 111 and linked ledger rows in 130/138/710.

In some embodiments, routing engine modules 801-809 operate in a closed-loop with the MILP/RL schedulers 201 and 221-226. Feasibility updates from 802-804 and downstream capacity forecasts (e.g., 1207) adjust admissible cross-facility actions prior to optimization, and post-solve outcomes recorded in ledgers 138/710 feed back to 803 so facility load vectors reflect realized transfers and realized utilization. For example, when sterile processing module 606 signals a late sterilization release at Facility X, feasibility masks 107 update locally and module 805 temporarily elevates Facility Y suitability for TRS-eligible robotic cases; ledger writer 807 records the advisory recommendation and governance console 235 surfaces the change for oversight under the active policy version.

FIG. 9 illustrates a perioperative optimization engine designed to integrate pre-operative readiness, intra-operative telemetry, and post-operative recovery into a unified perioperative state representation for surgical scheduling and capacity management. Operating within a de-identified compute plane 610 under de-identification and linkage boundary 109, the system maintains privacy compliance while interoperating with optimization controllers 201 and 221-226, constraint lattice elements 501-507, governance layers 231-235, and routing logic 801-809.

The process begins with a pre-operative readiness evaluator 901, which computes pre-operative readiness signal

$${ρ}_{k , t}^{p r e}$$

for each case k represented by nodes 101 using PHI-free feature vectors X[k] 108. For example, module 901 ingests lab completeness flags derived upstream via 121-125, imaging readiness indicators, and anesthesia/consult clearances encoded in node attributes 103 to produce a normalized readiness value in [0, 1]. In some embodiments, 901 gates low-readiness cases from near-term start windows by zeroing corresponding entries in feasibility masks 107; in other embodiments, 901 down-weights such cases via cost generator 204 so they are scheduled later.

Next, an intra-operative telemetry processor 902 processes real-time telemetry data, including anesthesia signals and surgical progress notes, to compute an intra-operative signal

$${ρ}_{k , t}^{intra}$$

and a duration-drift metric [symbol] relative to baseline durations encoded in X[k] 108. For example, module 902 detects a prolonged induction and computes a positive drift, which updates room-occupancy projections used by feasibility matrices 202 and informs turnover expectations used by room capacity model 505. In some embodiments, 902 also consumes structured events linked to conflicts 104 (for example, a robot “in-use” flag) and propagates late-release times to equipment feasibility mask 607 and combined feasibility operator 609.

Following surgery, a post-operative recovery predictor 903 computes a post-operative recovery/discharge readiness signal

$${ρ}_{k , t}^{post}$$

based on de-identified PACU telemetry, nursing notes, sedation levels, and pain assessments. For example, module 903 produces a recovery signal that increases as sedation and pain indicators normalize, signaling readiness to transfer to floor or ICU. In some embodiments, 903 feeds bed-type matching 1105 and capacity feasibility scoring 1107 so downstream unit placement is informed by both clinical readiness and capacity safety; in other embodiments, 903 informs discharge opportunity scores 1305 and discharge gating masks 1306 to accelerate bed release under unit pressure.

The outputs from modules 901-903 are fused by a unified perioperative state machine 904 into a perioperative state representation

$${z}_{t}^{peri} ,$$

which may incorporate

$${ρ}_{k , t}^{p r e} , {ρ}_{k , t}^{i n t r a} ,$$

[symbol]

and

$${ρ}_{k , t}^{post}$$

together with unit-level load indicators 1203/1207 and queue/assignment context from 301/205. For example, module 904 constructs a perioperative feature block including current room occupancy, remaining backlog derived from nodes 101, TRS values from 422, and capacity signals from 1106/1206, enabling decisions that reflect both clinical status and operational constraints. In some embodiments, 904 maintains separate feature channels per feasibility class 105 or room category to stabilize downstream policy evaluation performed by value/Q evaluator 305.

The Optimization Bridge 905 integrates these perioperative scores into the objectives and feasibility constraints of optimization controllers, such as MILP engine 201 and RL controller 221-226. For example, 905 increases delay penalties in 204 when intra-op drift 902 indicates late rooms and amplifies capacity terms when post-op scores 903 and census forecasts 1207 predict ICU strain. In some embodiments, 905 dynamically updates masks 107 for forthcoming slots based on 901-903 and recomputes combined feasibility 609; in other embodiments, 905 only adjusts weights in 204/225 while preserving hard feasibility defined by 501-507.

The TRS Integrator 906 further enhances the system by combining perioperative signals with Throughput Risk Scores (TRS) from 422/424. For example, 906 promotes high-TRS, high-readiness cases within the TRS-ordered queue 443 while deprioritizing coverage-unstable cases flagged by 1006. In some embodiments, 906 applies share-based gating 423 so only the top TRS tier with adequate 901 readiness enters add-on consideration; in other embodiments, 906 smooths rank changes with 452-454 to avoid disruptive queue oscillations after minor clinical updates.

To maintain traceability and compliance, the Ledger Writer 908 logs all perioperative state changes and parameter updates in a tamper-evident, PHI-free audit ledger 138/710. For example, 908 records the time-stamped transition from pre-op score 901 to intra-op drift 902 and the effect on feasibility masks 107, together with the active policy version 139 and the resulting assignment set from 205. In some embodiments, 908 cross-links entries to ledger 130 so data provenance from 121-129 is preserved end-to-end; in other embodiments, 908 attaches reason codes (for example, “PACU unstable”) for human override review in 235.

A UI enclave boundary 909 provides a secure interface for authorized users to access re-identified perioperative context. For example, module 909 displays patient context and perioperative trend summaries to clinicians after resolving DID linkage within the controlled enclave (e.g., via 129/705), while enforcing role-based access 713 and DLP controls 716 such that only PHI-free aggregates may leave the enclave 137/712. In some embodiments 909 supports human override actions (e.g., reassign room or defer case) and records those events via ledger writers 309/456 to append-only ledger 138; in other embodiments, 909 supports attorneys-eyes-only exports via secure export hooks 719 that include only de-identified artifacts (e.g., 108/107 and outcome summaries).

This perioperative optimization engine interacts with other system components, such as the MILP/RL controllers 201/221-226, TRS scoring 401-429, and capacity management modules 1101-1111, 1201-1209, and 1301-1309. By providing real-time, PHI-free updates to scheduling and capacity signals, the engine enables efficient, risk-aware, and privacy-preserving management of perioperative workflows. For example, when sterile processing signals a late instrument release via 606 and module 902 detects prolonged anesthesia, module 905 increases delay penalties in 204, module 201 re-solves under updated feasibility masks 107, modules 231/232 record corresponding metric changes, and governance console 235 surfaces the adjustment for oversight.

FIG. 10 illustrates a flowchart of a coverage-aware scheduling optimization engine operating within a de-identified compute plane 610 under boundary 109. The engine evaluates and prioritizes scheduling actions based on coverage readiness, administrative risk, and operational feasibility, ensuring compliance with privacy regulations while optimizing operational decisions. The flow interoperates with feasibility masks 107, conflict artifacts 104, MILP/RL controllers 201/221-226, TRS scoring 422/424, perioperative signals 901-903, policy versions 139, and audit ledgers 130/138/710.

The flow begins with a coverage rule extractor 1001, which parses and normalizes payer rules and coverage metadata into structured rule sets req_docsₖ and policy_metaₖ. For example, module 1001 encodes “authorization required for inpatient site-of-service” and “referral required for subspecialty consult” as machine-readable predicates. In some embodiments, module 1001 maps procedure codes and diagnosis codes to policy gates that can be referenced by feasibility masks 107; in other embodiments, module 1001 stores rule snapshots in the versioned policy store 139 with associated version identifiers so schedules and decisions can be reproduced against the exact rulebook in effect.

An authorization/referral status processor 1002 computes a current authorization, referral, and pre-certification status pa_status_{k,t} for each case k represented by node 101. For example, module 1002 marks a case “fully authorized through date X” or “authorization pending” and emits a status vector aligned to the discrete time grid produced by 124. In some embodiments, module 1002 cross-checks payer constraints from 1001 with documentation events in modules 121-125 to determine conditional authorizations; in other embodiments, module 1002 writes PHI-free status changes and associated version identifiers to one or more append-only ledgers (e.g., audit ledger 130 and/or decision/provenance ledgers 138/710) for traceability.

A documentation completeness evaluator 1003 compares present documentation doc_present_{k,t} against the payer requirements req_docsₖ to compute a documentation completeness indicator doc_{k,t}. For example, module 1003 verifies the presence of imaging reports and medical-necessity statements needed for a procedure and emits a binary or graded completeness score. In some embodiments, module 1003 contributes a documentation gate to feasibility masks 107 so documentation-incomplete cases are blocked from near-term slots; in other embodiments, module 1003 contributes a soft penalty term to cost generator 204 to defer documentation-risky cases while preserving hard feasibility.

An administrative cancellation risk model 1004 predicts a likelihood that a case is being canceled or delayed due to administrative issues. The model output

$${p}_{k , t}^{adm}$$

uses inputs including authorization status from 1002, documentation completeness from 1003, policy metadata from 1001, and historical denial or cancellation patterns captured in PHI-free ledgers (e.g., 138/710) to compute an administrative cancellation-risk score. For example, module 1004 assigns higher risk to out-of-network cases lacking current authorization. In some embodiments, module 1004 gates feasibility masks 107 when risk exceeds a policy threshold; in other embodiments, the score from 1004 is provided to reward shaping (e.g., module 223) to discourage high-risk scheduling actions.

A coverage feasibility score engine 1005 combines documentation completeness 1003, authorization status 1002, and policy metadata 1001 to compute a coverage feasibility signal

$${c}_{k , t}^{cov}$$

for each case anu candidate time slot. For example, module 1005 emits “coverage-feasible today” or “feasible in 48 hours pending referral,” aligned to discrete grid 124. In some embodiments, module 1005 contributes a multiplicative coverage gate to feasibility masks 107; in other embodiments, module 1005 supplies a graded coverage feasibility score cost to generator 204.

An operational feasibility score indicator 1006 integrates multiple factors-including coverage feasibility

$${c}_{k , t}^{cov}$$

from 1005, administrative risk

$${p}_{k , t}^{adm}$$

from 1004, throughout risk scores (TRS) from 422/424, and perioperative readiness signals from 901-903—to compute an overall operational feasibility score

$${c}_{k , t}^{op}$$

for each case and candidate time slot. For example, module 1006 increases feasibility for a fully authorized case with high TRS, complete documentation, and strong pre-op readiness from 901, while down-weighting a pending-authorization case during ICU surge indicated by 1207. In some embodiments, module 1006 incorporates downstream bed feasibility 1106 and capacity safety scoring 1107 so scheduling reflects both administrative readiness and unit pressure; in other embodiments, module 1006 exposes component breakdowns to governance console 235 for transparency.

A MILP/RL optimization interface 1007 applies coverage-based feasibility artifacts and operational feasibility scores to constrain and shape the set of scheduling actions presented to MILP engine 201 and RL controller 221-226. For example, module 1007 zeros out assignments within feasibility masks 107 when module 1005 indicates coverage infeasibility and adjusts cost coefficients in cost generator 204 using

$${c}_{k , t}^{op}$$

to prefer authorized and documentation-complete cases. In some embodiments, module 1007 throttles RL exploration (e.g., via 225) when administrative risk

$${p}_{k , t}^{adm}$$

spikes; in owner embodiments, module 1007 triggers a re-solve in MILP engine 201 when authorization status transitions to approved.

An authorized UI enclave 1009 provides a secure environment for displaying coverage-aware scheduling recommendations to authenticated users. Re-identification occurs only within enclave 1009 under role-based access control 713 and UI boundary controls associated with enclave 137, ensuring privacy while enabling clinicians and schedulers to act on system recommendations. For example, module 1009 renders a schedule annotated with “authorization approved” or “docs incomplete,” with patient context resolved from DID linkage within the controlled enclave (e.g., via re-identification mechanisms described with reference to FIG. 7), while DLP controls 716 prevent PHI egress.

To maintain traceability and compliance, coverage-aware computations and decisions are logged as PHI-free rows in one or more append-only ledgers (e.g., audit ledger 130 and/or decision/provenance ledger 138/710). For example, 1001-1006 write configuration versions and computed scores; module 1007 records applied coverage gates and resulting schedule effects; and module 1009 records re-identification display events linked to the corresponding de-identified ledger entries. In some embodiments, the versioned policy store 139 maintains coverage weights, thresholds, and rule snapshots so optimization behavior can be reproduced against specific rulebooks; in other embodiments, governance console 235 surfaces coverage-driven changes in utilization, cancellations, and denial rates for oversight.

This flow integrates coverage-related constraints into the scheduling process so that cases with higher administrative readiness and lower cancellation risk are preferentially scheduled while disruptions from incomplete documentation or authorization are reduced. For example, when module 1002 updates a pending authorization to approved and module 1003 signals complete documentation, module 1007 unblocks near-term slots by updating feasibility masks 107, MILP engine 201 re-solves the day schedule using updated costs from 204, and RL modules 221-226 learn to favor coverage-stable actions under the active policy version in store 139.

FIG. 11 illustrates a bed, census, and capacity assignment engine that translates de-identified case features X[k] 108, throughput risk signals from 422/424, and census inputs from 1201-1209 into downstream bed feasibility 1106 and capacity feasibility scoring 1107 for scheduling and routing decisions. The engine operates entirely within the de-identified compute plane 610 under boundary 109 and interoperates with upstream modules including conflict artifacts 104, feasibility masks 107, MILP/RL controllers 201 and 221-226, discharge logic 1301-1309, and audit ledgers 130/138/710, while any re-identification for presentation occurs only at the UI enclave 137/611.

A bed type predictor 1101 uses de-identified inputs such as procedure class, acuity proxies, ICU propensity features, and perioperative signals 901-903 to infer a required downstream bed type and an acceptable bed-type set [symbol] _{type} (k) for each case k. For example, module 1101 predicts that a higher-acuity cardiac case should route to ICU initially, whereas a low-acuity arthroscopy routes to a medical/surgical bed. In some embodiments, module 1101 is a rules engine mapping procedure categories and risk indicators to a bed-type set; in other embodiments, module 1101 is a learned classifier trained on historical post-operative placements keyed by DID and recorded in decision/provenance ledgers 138/710.

A length-of-stay (LOS) forecasting engine 1102 produces LOS predictions [symbol] and, optionally, unit-specific LOS distributions for the predicted bed types. For example, module 1102 estimates a median LOS of three days for a predicted ICU placement with a longer tail under the observed case-mix. In some embodiments, module 1102 conditions LOS estimates on TRS 422/424 and perioperative drift from 902; in other embodiments, module 1102 leverages historical LOS distributions keyed by service line and procedure clusters encoded in X[k] 108.

A census integration pipeline 1103 ingests ADT events 1201, bed occupancy and staffing signals, isolation and cohorting rules 1205, and unit metadata to compute de-identified bed-level occupancy occ_{b,t} and staffed capacity indicators staffed_{b,t}. For example, module 1103 maintains time-indexed occupancy indicators and staffed status for each bed and propagates isolation availability flags to unit-level feasibility signals 1206. In some embodiments, module 1103 normalizes events to the discrete time grid 124 to align with upstream feasibility evaluation and solver intervals; in other embodiments, module 1103 aggregates bed-level signals into facility-level matrices used by routing modules 801-809.

A bed availability evaluator 1104 converts census and staffing inputs into bed availability flags avail_{b,t}. For example, module 1104 marks a bed as unavailable when occupied or unstaffed for a slot and available when both unoccupied and staffed. In some embodiments, module 1104 incorporates planned maintenance blocks and temporary closures captured in unit metadata; in other embodiments, module 1104 anticipates near-term availability using scheduled discharges indicated by discharge modules 1301-1309.

A bed-type to case matching module 1105 evaluates clinical compatibility between each case's acceptable bed-type set [symbol] _{type} (k) and candidate beds. For example, module 1105 filters out step-down beds for a case whose acceptable set contains only ICU and medical/surgical types. In some embodiments, module 1105 also enforces special-unit requirements such as telemetry capability or isolation criteria sourced from 1205; in other embodiments, module 1105 applies unit-specific cohorting or placement rules configured per unit.

A composite bed feasibility mask

$${mask}_{k , b , t}^{b e d} .$$

1106 is constructed by combining bed-type compatibility from 1105, unit-level feasibility (e.g., 1206), and bed availability from 1104 for each case k, bed b, and time slot t. For example,

$$m a s {k}_{k , b , t}^{b e d} = 1$$

when bed b is clinically appropriate for case k, staffed and unoccupied at time t, and permitted by unit-level isolation and surge rules. In some embodiments, module 1106 projects feasibility across a predicted LOS window from 1102 to reduce mid-stay conflicts; in other embodiments, module 1106 distinguishes hard feasibility (binary) from soft feasibility (graded confidence) for use by cost generator 204 while preserving hard constraints in solver 205.

A capacity feasibility scoring engine 1107 aggregates bed-level feasibility into a capacity-aware score

$${c}_{k , t}^{cap}$$

for each case and start time. For example, module 1107 increases

$${c}_{k , t}^{cap}$$

when many feasible beds exit over the LOS horizon and decreases

$${c}_{k , t}^{cap}$$

when forecasts indicate ICU overflow risk 1207. In some embodiments, module 1107 computes an overflow risk term from forecasted census and staffed capacity signals 1203/1204 and combines it with post-operative readiness 903 and TRS 422/424 to produce a bounded score in [0, 1]. In other embodiments, module 1107 outputs both a scalar score and component breakdowns (e.g., feasible bed count, overflow probability, readiness level) for governance console 235.

An integration interface 1108 connects bed and capacity signals to perioperative and TRS logic. For example, interface 1108 routes post-operative readiness signals from 903 and TRS signals from 422/424 to bias ICU versus floor placement when both are clinically acceptable. In some embodiments, interface 1108 exposes capacity feasibility score 1107 to RL reward shaping module 223 so actions that increase overflow risk receive lower reward; in other embodiments, interface 1108 provides a capacity-aware penalty term to MILP cost generator 204 to deter schedules that saturate constrained units.

An integration interface 1109 connects capacity constraints and overflow signals to the multi-facility routing engine 801-809. For example, interface 1109 signals when a local ICU has low slack capacity so routing evaluator 805 uplifts suitability for alternative facilities with healthier ICU forecasts 1207. In some embodiments, interface 1109 tags feasibility changes with an active policy version identifier from store 139 for reproducibility; in other embodiments, interface 1109 streams de-identified capacity matrices to routing so cross-facility decisions remain consistent with near-term load projections and recorded in ledgers 138/710.

All computations in FIG. 11 occur within the de-identified compute plane 610 using deterministic linkage identifiers (DIDs) and PHI-free feature vectors X[k] 108, and all decisions are recorded in one or more append-only ledgers 130/138/710. For example, the engine logs hashes of bed feasibility artifacts

$$( e . g . , mas {k}_{k , b , t}^{b e d} 1106 ) ,$$

LOS forecast parameters from 1102, and the inputs used to compute capacity feasibility scores 1107 for each scheduling cycle. In some embodiments, the UI enclave 137/611 renders recommended bed assignments and capacity risks after re-identifying patients for authorized staff, with DLP controls 716 preventing PHI egress; in other embodiments, only de-identified capacity dashboards are shown outside the enclave for operational monitoring.

In one example, a high-TRS vascular case is predicted by 1101 to require an ICU bed with an LOS estimate of two days from 1102. Current ICU census from 1203 indicates 95% occupancy with staffing from 1204 at 100%, and forecasts from 1207 indicate elevated overflow risk if two additional ICU admissions arrive during the planned window. The composite bed feasibility mask 1106 identifies only one ICU bed feasible across the LOS horizon, and 1107 yields a low-capacity feasibility score

$${c}_{k , t}^{cap} .$$

The integration interface 1108 increases MILP costs via 204 for start times that would admit during a surge window, and the scheduler 201 selects an earlier slot when

$${c}_{k , t}^{cap}$$

is higher. If no feasible slot exists, 1109 signals routing evaluator 805 to elevate a nearby facility with lower ICU load, while ledger writer 807 logs the advisory recommendation for governance review by console 235.

In some embodiments, 1106 and 1107 incorporate dynamic discharge opportunities 1305-1307 so bed feasibility reflects anticipated releases and uncertainty in discharge timing. In some embodiments, 1101 and 1102 are updated in real time by intra-operative drift 902 and post-operative recovery 903 so downstream capacity and placement decisions adapt as cases progress. In some embodiments, the engine emits shadow prices or scarcity indicators for constrained beds, which 207 and/or reward module 203 can use to discourage schedules that consume scarce unit capacity; in other embodiments, the engine supports what-if queries that assess the impact of additional staffed beds or alternative staffing patterns on feasibility 1106 and capacity scores 1107.

FIG. 12 illustrates a census integration and capacity forecasting engine that maintains a de-identified, time-indexed view of bed occupancy, unit-level census, and capacity across one or more facilities, and projects those quantities forward over a configurable horizon. The engine operates entirely within the de-identified compute plane 610 under de-identification linkage boundary 109 and interoperates with bed feasibility and capacity scoring 1106/1107, scheduling controllers 201/221-226, routing logic 801-809, discharge optimization 1301-1309, and append-only ledgers 130/138/710, while any re-identification for presentation occurs only within UI enclave 137/611.

An ADT ingestion interface 1201 receives HL7/ADT and related feed messages (admissions, transfers, discharges, holds, cancellations) and normalizes them into de-identified events. For example, 1201 ingests an “admit to ICU” event and a “transfer to step-down” update, mapping both to the discrete time grid 124. A patient-bed-unit mapping module 1202 maps each event to a DID-tokenized patient, a de-identified bed token, and a unit token, updating bed-level occupancy and unit-level census within the de-identified compute plane. For example, 1202 sets an ICU bed occupancy flag to 1 for the duration implied by the event and updates the ICU census count accordingly.

A census normalization and aggregation engine 1203 computes unit-level census and facility-level aggregates from bed-level occupancy indicators. For example, engine 1203 produces per-unit time series of occupied beds and summarizes facility census totals for dashboards and policy checks. In some embodiments, engine 1203 clips occupancy to staffed capacity module 1204; in other embodiments, engine 1203 records both physical capacity and staffed capacity so downstream modules can discriminate between physical availability and staffing constraints.

A capacity and staffing integration module 1204 combines bed metadata, surge rules, and staffing signals to derive physical bed capacity, staffed capacity, and unit load metrics. For example, module 1204 converts nurse-to-patient ratio plans into staffed bed counts and marks surge activation windows that temporarily tighten capacity. In some embodiments, module 1204 encodes seasonal or event-driven staffing patterns; in other embodiments, module 1204 ingests real-time staffing feeds to update staffed capacity on the grid 124.

An isolation and special-unit constraints module 1205 evaluates isolation requirements, cohorting rules, gender placement, telemetry capability, and special observation criteria to produce isolation availability indicators that constrain where cases may be placed. For example, module 1205 marks “airborne isolation unavailable” for a given unit when all negative-pressure rooms are occupied. In some embodiments, module 1205 encodes cohort rules (e.g., pediatric vs adult, gender placement); in other embodiments, module 1205 tracks temporary isolation closures due to maintenance.

A unit-level feasibility mask generator 1206 combines bed-type compatibility (from 1105), isolation signals from module 1205, surge flags, and special-unit policies to construct unit-level feasibility masks for each case and unit over time. For example,

$${mask}_{k , u , t}^{unit} = 1$$

indicates that unit u can accept case k at time t under isolation and staffed-capacity rules. In some embodiments, generator 1206 writes mask hashes to ledger 138/710 for audit; in other embodiments, generator 1206 streams updated masks to schedulers 201 and 221-226 when isolation policy changes.

A LOS-aware census forecasting engine 1207 uses LOS predictions from 1102, historical patterns, and planned admissions to produce forward-looking census, capacity, and load forecasts over a configurable horizon. For example, 1207 projects ICU occupancy for the next 48 hours by combining current ICU census 1203, scheduled surgical admits from MILP engine 201, and expected discharges signaled by discharge opportunity scores and forecasted bed-release events in 1305 and 1307. In some embodiments, engine 1207 conditions forecasts on TRS 422 and perioperative drift 902; in other embodiments, engine 1207 blends historical time-of-day load profiles with scheduled intake to smooth short-term spikes.

A multi-hospital census matrix exporter 1208 assembles these signals into network-wide census and capacity matrices across hospitals and units, enabling FIG. 8 and FIG. 11 to detect overflow and route cases between facilities when multi-site operation is enabled. For example, 1208 provides a matrix of forecasted ICU load ratios per facility that routing evaluator 805 consumes to bias routing away from saturated sites. In some embodiments, 1208 tags exported matrices with policy version identifiers from versioned policy store 139 and with hashes of relevant masks 107/1206 to ensure reproducibility; in other embodiments, 1208 streams updates via an event bus 717 to downstream consumers.

All computation occurs within the de-identified compute plane 610, where patients, beds, and units are represented by DID tokens k ∈ [symbol] and PHI-free indices, and all decisions and forecasts are recorded in append-only ledgers 130/138/710. For example, ledger 710 logs a census forecast vector, staffed capacity parameters, and isolation mask hashes produced by module 1205 and generator 1206 for each cycle. In some embodiments, the UI enclave 137/611 renders unit-level capacity dashboards after re-identifying patient context for authorized staff, with DLP controls 716 preventing PHI egress; in other embodiments, only de-identified capacity aggregates are exposed outside the enclave.

For example, a surgical service plans three ICU-requiring cases for the afternoon. ADT events 1201 reflect two morning ICU discharges and one transfer to step-down; mapping 1202 updates ICU bed occupancy. Normalization 1203 and capacity integration 1204 indicate staffed ICU capacity will be at 90% by noon. Isolation constraints 1205 show airborne rooms are fully occupied. Unit feasibility 1206 blocks airborne-precaution cases from ICU placement at 13:00. Forecasting 1207 projects ICU overflow risk if two additional ICU admits arrive between 14:00-16:00. The census matrix exporter 1208 signals routing evaluator 805 to uplift suitability for a nearby facility with lower ICU load, while the MILP engine 201 increases capacity-penalty weights via cost generator 204 for admits in the surge window. Ledger 138 and/or 710 records the forecasts, masks, and policy adjustments for governance review in console 235.

In some embodiments, generator 1206 and forecasting engine 1207 incorporate dynamic discharge opportunities 1305-1307 so unit feasibility and forecasts reflect anticipated bed releases and uncertainty in discharge timing. In some embodiments, 1201-1208 operate on the discrete time grid 124 to align with feasibility evaluation and solver intervals, ensuring consistent gating of actions by feasibility masks 107 and capacity signals 1107 across the stack.

FIG. 13 illustrates a discharge-based optimization engine that computes a Discharge Opportunity Score (DOS) for each de-identified inpatient and time slot, then converts selected opportunities into forecasted bed-release events that feed back into the census and scheduling layers. The engine operates entirely within the de-identified compute plane 610 under de-identified linkage boundary 109 and interoperates with perioperative readiness 901-903, TRS scoring 422/424, bed/census and capacity modules 1101-1109/1201-1208, scheduling controllers 201/221-226, constraint lattice 501-507, audit ledgers 130/138/710, and governance components 231-235, while any re-identification for user-facing presentation occurs only at the UI enclave 137/611.

A clinical readiness extraction submodule 1301 computes clinical readiness scores ready_clinical_{k,t} for inpatients from PHI-free signals. For example, 1301 aggregates de-identified labs and vitals from interface 1201 and engine 1203, consult completion and stability indicators from perioperative signals 901-903, and notes-derived embeddings into a bounded clinical readiness score in [0, 1]. In some embodiments, submodule 1301 applies rule-based gates for mandatory conditions (e.g., afebrile for 24 hours, stable hemodynamics); in other embodiments, 1301 uses learned models keyed to feature vectors X[k] 108 and logs model versions and hashes to 138/710.

A unit utilization readiness module 1302 derives utilization pressure scores ready_util_{u,t} using census and capacity signals. For example, 1302 ingests unit-level census from 1203, staffed capacity from 1204, isolation availability from 1205, and short-horizon forecasts in engine 1207 to compute a pressure score reflecting the operational benefit of freeing a bed now versus later. In some embodiments, 1302 encodes surge thresholds and diversion policies from versioned policy store 139; in other embodiments, 1302 weights pressure by time-of-day load patterns and block schedules linked to resource templates 106.

A coordination readiness assessment 1303 aggregates logistics-related indicators into a coordination readiness score ready_coordₖ. For example, 1303 encodes post-acute acceptance (SNF/rehab), PT/OT evaluation status, transportation scheduling, durable medical equipment readiness, and case management disposition, expressed as PHI-free flags. In some embodiments, 1303 pulls de-identified task completion states from workflow systems integrated via 122 and writes changes to ledger 130/138/710 for audit; in other embodiments, 1303 assigns graded scores to reflect partial completion or expected lead times.

A TRS-based discharge safety mask 1304 applies a policy-defined threshold

$${τ}_{TRS}^{dis}$$

over TRS 422 and selected clinical signals to block unsafe or high-risk discharges. For example, 1304 zeros out discharge eligibility when TRS exceeds a facility-configured threshold or when isolation constraints from 1205 prevent safe transfer. In some embodiments, 1304 includes condition-specific gates (e.g., cardiac telemetry requirements) and consult prerequisites; in other embodiments, 1304 is time-window specific, tightening safety during surge periods indicated by 1207.

A DOS computation block 1305 fuses clinical readiness 1301, unit utilization pressure 1302, and coordination readiness 1303 into a scalar Discharge Opportunity Score DOS_{k,t} for each patient and time step. For example, 1305 computes a weighted sum normalized to [0, 1], with weights read from versioned policy store 139 and logged to ledger 138/710. In some embodiments, 1305 applies monotone transforms to stabilize score ranges across units; in other embodiments, 1305 uses learned fusion calibrated via horizon outcomes 431/432 (FIG. 4D) and records calibration deltas in ledger 138.

A discharge feasibility gate 1306 combines Discharge Opportunity Score from 1305, the safety mask

$${mask}_{k , t}^{dis}$$

1304, and unit-level feasibility and capacity constraints to determine which discharge actions dis_{k,t} are admissible at the current time. For example, 1306 enforces that a candidate discharge does not violate isolation, cohorting, and placement constraints evaluated by module 1205 and generator 1206 and respects minimum clinical thresholds encoded by 1301/1304, outputting a binary admissibility flag per patient/time. In some embodiments, 1306 returns a tuple separating hard admissibility (binary) from soft feasibility (graded) that is consumed by cost generator 204 and RL reward logic from 223; in other embodiments, 1306 outputs a “defer” indicator with next-best feasible time windows aligned to the time grid 124.

A forecasted bed-release event module 1307 converts accepted discharge decisions into de-identified bed-release signals that update census trajectories and downstream scheduling. For example, 1307 emits a release event tagged to a de-identified bed token and unit index with release time snapped to grid 124, and forwards the event to census components 1203 and 1207 to refresh occupancy projections and overflow risk estimates used by 1107. In some embodiments, 1307 attaches confidence bounds when coordination completion is uncertain; in other embodiments, 1307 batches or rate-limits releases to reduce forecast instability.

A capacity and scheduling integration interface 1308 routes discharge signals and DOS-related artifacts to optimization controllers 201 and 221-226 and constraint artifacts 501-507. For example, 1308 raises capacity-aware penalty terms in cost generator 204 for surgical starts that coincide with low expected release and reduces such penalties when a block of release is imminent based on 1307. In some embodiments, 1308 updates feasibility masks 107 for forthcoming intervals when 1207 indicates capacity relief; in other embodiments, 1308 provides scarcity indicators or shadow prices for constrained units to reward shaping logic in 223 while preserving hard feasibility under 501-507.

An output-to-solver and governance interface 1309 provides DOS values, safety masks, accepted discharge actions, and forecasted bed releases to scheduling and governance layers. For example, 1309 writes a hash-chained ledger entry to 138/710 that records inputs (including hashes over 108 and relevant 1203/1207 signals), active configuration identifiers from versioned policy store 139, and outputs (accepted discharge actions and 1307 bed-release events), and surfaces an operational summary to governance console 235 for approval, review, or rollback when thresholds or weights change. In some embodiments, 1309 exposes de-identified dashboards outside the UI enclave showing discharge cadence and projected unit relief; in other embodiments, 1309 supports scenario testing (“what-if 10:00 discharges”) with the scenario inputs and outputs logged to 138/710 for reproducibility.

All discharge computations in FIG. 13 occur on PHI-free artifacts—feature vectors X[k] 108, census signals 1201-1208, masks 107/1206—and any patient re-identification for display or override occurs only within UI enclave 137/611 under role-based access control 713 and DLP enforcement 716. For example, case managers review re-identified discharge recommendations within 137, accept or defer actions, and operator overrides are recorded in decision ledger 138 as operator-authored entries linked to upstream provenance in ledger 130.

For example, a medical ICU unit exhibits high utilization pressure in 1203/1207 for the afternoon window. Clinical readiness 1301 identifies three stable patients with normalized readiness above 0.8; and coordination readiness 1303 indicates that two have confirmed post-acute acceptance and transportation arranged; The discharge safety mask

$${mask}_{k , t}^{dis}$$

1304 blocks one patient due to elevated risk. DOS in 1305 ranks the remaining two patients highest, and gate 1306 admits both discharge actions as feasible. Bed-release events 1307 update 1207, increasing predicted ICU slack; 1308 reduces capacity penalties in cost generator 204 for an early vascular case and updates forthcoming feasibility entries in 107 to reflect safer admission intervals; MILP engine 201 re-solves to increase throughput without breaching unit constraints, and ledger 138/710 records the discharge-to-scheduling chain for governance review in console 235.

FIG. 14 shows an AI failure handling and safe-mode override engine that monitors solver outputs for safety and compliance, detects anomalies, initiates conservative fallback behavior when required, and records a tamper-evident audit trail for each cycle and human override. The engine operates within the de-identified compute plane 610 under de-identification linkage boundary 109 and interoperates with feasibility masks 107, conflict artifacts 104, schedulers 201 and 221-226, governance modules 231-235, and ledgers 130/138/710, while any re-identification for user-facing presentation occurs only in the UI enclave 137/611.

An input and context collector 1401 aggregates de-identified solver inputs, feasibility masks, TRS values 422, capacity forecasts 1207, and discharge constraints 1306 into a normalized context representation for safety evaluation and logging. For example, 1401 bundles an assignment output summary from solver core 205, hashes for masks 107, capacity signals from 1107, and the active policy version identifier from 139 into a compact PHI-free context record. In some embodiments, 1401 enriches the context with violation metrics 523 and exploration throttles from policy parameter output 225; in other embodiments, 1401 includes non-identifying reason codes emitted by 601/604/607 for staff or equipment infeasibility.

A safety pre-validation module 1402 applies policy-defined safety bounds (“SafeRegion”) to each candidate schedule before any output is accepted. For example, 1402 rejects schedules that admit multiple ICU-requiring cases into a forecasted surge window indicated by 1207 or schedules that violate TRS-based discharge safety constraints enforced by 1304/1306. In some embodiments, 1402 enforces infection-control sequencing rules form precedence and clinical rule set 506 and anesthesia supervision ratios evaluated in the anesthesia coverage evaluator in 604; in other embodiments, 1402 applies coverage gates 1005/1006 to block administratively infeasible actions prior to execution.

A solver output and hash encoder 1403 converts a proposed schedule or admissible action set [symbol] ^{feas} (sₜ) into a PHI-free representation and computes an output hash used for safe-region comparison and for ledger storage. For example, 1403 hashes decision variables 508, referenced mask identifiers 501/107, and active cost configuration identifiers from cost generator 204 into hashed outputs stored alongside hashed inputs, and the current policy version identifiers from policy store 139. In some embodiments, 1403 signs hashes using cryptographic keys managed by KMS/HSM 716 keys; in other embodiments, 1403 canonicalizes outputs prior to hashing to stabilize comparisons across replays.

A failure detection and anomaly flag module 1404 fuses SafeRegion evaluations and safety check results to produce an abnormal-output flag indicating whether the system should enter a safe-mode fallback. For example, 1404 sets the flag if ICU overflow probability derived from 1107 exceeds a policy threshold given the proposed admissions, or if repeated violations of anesthesia supervision ratios 604 are detected. In some embodiments, 1404 monitors drift in conflict density 104 and queue instability metrics from 451-455; in other embodiments, 1404 triggers based on elevated override frequency recorded in ledger 138.

A mode controller 1405 maintains an operating mode state, switching from normal operation to fallback mode when the abnormal-output flag is raised and coordinating a controlled return to normal after remediation. For example, 1405 suspends RL exploration 221-226 and locks policy updates in versioned policy store 139 when entering fallback and resumes adaptive updates only after violations subside for at least N consecutive cycles. In some embodiments, 1405 supports graded safe modes (e.g., exploration freeze only versus full conservative scheduling); in other embodiments, 1405 requires governance console 235 approval before exiting fallback.

A safe-mode MILP scheduler 1406 generates a conservative, deterministic schedule under fallback mode using a restricted MILP configuration that enforces hard feasibility and applies risk-averse objective weights. For example, 1406 prioritizes early starts for high-TRS cases with confirmed bead feasibility in 1106 and blocks elective starts during surge windows indicated by forecasting engine 1207. In some embodiments, 1406 pins execution to specific policy versions in 139 and mask versions in 107 for reproducibility; in other embodiments, 1406 reduces utilization targets and overtime risk to restore safety margins.

An override and UI interface 1409 surfaces flagged events, fallback schedules, and recommended actions to authorized users within the UI enclave boundary 137, records manual overrides as ledger entries, and ensures that any re-identification occurs only within 137/611. For example, 1409 displays a fallback schedule annotated with non-identifying reason codes (e.g., “ICU overflow risk,” “anesthesia ratio exceeded”), allows an authorized coordinator to defer an elective case, and logs the override decision in decision ledger 138 as an operator-authored principal entry. In some embodiments, 1409 captures structured feedback categories and tags policy rollback requests for governance console 235; in other embodiments, 1409 limits override scope to designated windows, resources, or service lines defined by policy in 139.

A policy and version management unit 1407 quarantines suspect configurations, enables rollback to a last-validated version, and records version transitions for governance. For example, 1407 deactivates a recently updated penalty or weight configuration when repeated SafeRegion violations occur and restores a prior validated policy version recorded in 139/138. In some embodiments, 1407 supports model freeze and reintegration workflows consistent with 1508-1509; in other embodiments, 1407 requires explicit governance console 235 approval before promoting or demoting versions.

An append-only audit ledger writer 1408 writes each decision cycle into an immutable, chain-linked ledger row that contains a principal identity class, an inputs hash, a policy version identifier, a previous hash, and an output hash, all in PHI-free form. For example, 1408 appends [symbol] principal=“system”, inputs_hash, policy_version=“v6.0”, prev_hash, out_hash [symbol] and cross-links to earlier provenance entries in ledger 130. In some embodiments, 1408 records SafeRegion status, mode indicators (e.g., “normal” versus “fallback”), violation metrics 523, and override details from 1409; in other embodiments, 1408 exports de-identified summaries to governance console 718 for compliance review.

For example, a day schedule proposes three ICU-requiring cases between 14:00-16:00, while 1207 forecasts ICU overflow risk and 1107 signals low-capacity feasibility. Module 1402 flags the schedule as outside SafeRegion, 1404 raises the abnormal-output flag, and 1405 enters fallback mode. Scheduler 1406 produces a conservative plan that advances a high-TRS case into a morning slot and defers elective starts; 1409 presents the plan in the UI enclave 137 with annotated rationale, and an authorized coordinator accepts a deferral and requests cross-facility evaluation via 801-809 for a remaining case. Ledger writer 1408 records the cycle, the fallback, and the override, and 1407 quarantines the aggressive configuration and rolls back to a prior policy version for continued operation. Governance console 235 later reviews linked entries in 138/710 entries and approves adjusted limits recorded in 139.

In some embodiments, the failure handling engine interoperates with policy update logic in 232 to increase penalties for repeated violations while preserving hard feasibility under 501-507. In some embodiments, SafeRegion bounds are versioned and audited alongside feasibility masks 107 and policy versions 139 to enable exact reconstruction of safety decisions. All operations remain PHI-free within 610/609, with any patient context displayed only in the UI enclave 137/611 under role-based access control 713 and egress controls 716.

FIG. 15 illustrates an AI model selection and governance dashboard that evaluates candidate solver models using PHI-free performance metrics metrics[m], compatibility checks against active feasibility constraints, and risk and drift signals derived from the append-only ledgers and manages which model and policy version is active for operation. The dashboard operates within the de-identified compute plane 610 under de-identification linkage boundary 109 and interoperates with optimization engines 201 and 221-226, constraint lattice elements 501-507, resource feasibility modules 601-609, TRS/DOS layers 401-467 and 1301-1309, and ledgers 130/138/710, while any re-identification for presentation occurs only in the UI enclave 137/611.

A model evaluation metrics module 1501 aggregates de-identified downstream performance metrics metrics[m] for each candidate model. For example, 1501 summarizes delay error, on-time starts, overtime minutes, throughput, capacity-aware accuracy derived from 1107/1207, TRS stability derived from 421-429, and DOS alignment derived from 1305-1309, using ledger entries 130/138/710 tied to recent solver runs. In some embodiments, 1501 computes service-line specific metrics to reflect heterogeneous constraints captured by 104/105/107; in other embodiments, 1501 normalizes metrics per planning window using the time grid 124 to enable fair comparisons.

A compatibility analysis engine 1502 verifies that each candidate model respects the active constraint lattice and feasibility masks. For example, 1502 checks that proposed schedules remain consistent with feasibility masks 107, staff credentials and supervision rules 603/604, equipment readiness 607, room capacity rules 505, precedence and sequencing rules 506, and coverage gates 1005/1006. In some embodiments, 1502 performs spot replays over recorded mask sets stored in graph store/cache 111 to confirm hard feasibility under 507; in other embodiments, 1502 validates configuration toggles and facility policy constraints in 608 prior to model eligibility.

A ledger feed and drift ingestion module 1503 consumes SafeRegion violations, fallback events, overrides, and policy transitions from ledgers 138/710 to compute risk and history signals. For example, 1503 derives drift signatures from repeated boundary breaches flagged by failure detection in 1404, counts fallback cycles maintained by 1405, and tallies override patterns recorded by override and UI interface 1409, producing risk indicators that penalize unstable models. In some embodiments, 1503 correlates violations to constraint classes 501-506 to isolate root causes; in other embodiments, 1503 tags history with policy versions in 139 and mask hashes in 107 for artifact-level reconstruction.

A model selection score computation block 1504 computes, for each candidate model m, a governance-weighted Model Selection Score score[m], from metrics[m] (e.g., from 1501) compatibility [m] (e.g., from 1502), and risk_score[m], and model_history[m] (e.g., from 1503) according to governance weights w^{gov}. In one embodiment:

$$score [ m ] = {w}_{1}^{g o v} \cdot metrics [ m ] + {w}_{2}^{g o v} \cdot compatibility [ m ] + {w}_{3}^{g o v} \cdot risk\_score [ m ] + {w}_{4}^{g o v} \cdot model\_history [ m ]$$

where metrics[m] aggregates PHI-free performance measures (e.g., delay error, TRS/DOS alignment, unit balance), compatibility[m] reflects respect for the constraint lattice and masks, risk_score[m] summarizes SafeRegion and fallback behavior, and model_history[m] summarizes drift, quarantines, and prior deployments, with w^{gov} configured by institutional governance preferences.

A policy-version update controller 1507 activates the selected model and coordinates solver rollout. For example, 1507 updates the active policy version 139, propagates updated cost weights to 204, exports exploration limits to RL via 225, pins mask references 107 for reproducibility, and writes the activation event to ledger 138/710. In some embodiments, 1507 supports staged rollout with rollback triggers tied to SafeRegion signals from 1404; in other embodiments, 1507 runs canary comparisons against a prior active model.

Quarantine and model-freeze logic 1508 blocks unstable models from re-selection until remediated. For example, 1508 quarantines models associated with repeated overflow or supervision violations despite policy tuning, preventing automatic activation. In some embodiments, 1508 freezes models following governance review via 235 while preserving metadata for transparency; in other embodiments, 1508 escalates quarantines when override frequency exceeds a policy-defined threshold over N cycles.

A reintegration module 1509 manages the return of approved models to the candidate set. For example, 1509 restores a quarantined model after updated TRS weights or penalty coefficients reduce prior violations, records the reintegration event in 138/710, and refreshes eligibility for future selection. In some embodiments, 1509 requires a compatibility re-check via 1502 against current masks 107 and constraint classes 501-506; in other embodiments, 1509 mandates a minimum shadow period with performance acceptance tests measured by 1501.

A governance interface 1505 presents recommended models and their component scores to authorized users within the UI enclave boundary 137, enabling approval, rejection, or alternative selection. For example, 1505 displays candidate rankings, component breakdowns (metrics/compatibility/risk score/model history), recent violation summaries, and estimated impacts on throughput and capacity; an authenticated user can approve the recommended model or select an alternative with recorded rationale. An override executor 1506 applies governance overrides by activating the selected alternative model and records the override decision in ledger 138. In some embodiments, 1506 requires justification categories for audit; in other embodiments, 1506 enforces role-based limits on override authority and conditions defined by policy in 139.

All model identities, scores, and selection outcomes are recorded in PHI-free form in the append-only ledgers 130/138/710 to provide a complete auditable record of AI governance decisions. For example, the dashboard writes a chain-linked row containing the recommended model identifier, the governance selection outcome, the policy-version change, referenced mask hashes 107, and hashes of the performance and compatibility summaries used for the decision. In some embodiments, the dashboard emits de-identified governance reports to console 718; in other embodiments, selection artifacts are snapshotted by forensic snap shotter 720 for forensic reproducibility together with model hashes, mask hashes, and policy version identifiers.

While exemplary embodiments are described above, it is not intended that these embodiments describe all possible forms encompassed by the claims. The words used in the specification are words of description rather than limitation, and it is understood that various changes can be made without departing from the spirit and scope of the disclosure. As previously described, features of various embodiments may be combined to form further embodiments that may not be explicitly described or illustrated. While some embodiments may be described as providing advantages or being preferred relative other embodiments those of ordinary skill in the art will recognize that one or more features may be compromised to achieve desired overall system attributes depending on the specific application of the implementation. These attributes can include, but are not limited to cost, strength, durability, life cycle cost, marketability, appearance, packaging, size, serviceability, weight, manufacturability, ease of assembly, etc. As such, to the extent any embodiments are described as less desirable than other embodiments or prior art implementations with respect to one or more characteristics, these embodiments are not outside the scope of the disclosure and can be desirable for particular applications.

What is claimed is:

1. A computer-implemented method for optimizing clinical and operational workflows in healthcare environments, comprising:
receiving data indicating one or more resources from the healthcare environments;
receiving data indicating one or more records related to patient information;
generating, for each of the data indicating one or more of the records, a de-identified feature representation that excludes direct patient identifiers of the patient information;
constructing a model based on the generated de-identified feature representations, the data indicating the records, and the data indicating the resources;
deriving, for each of the records, weights from the model, wherein the weights represent a numerical value of whether one or more of the resources are available for assignment at corresponding time intervals under resource constraints of the resources;
assigning, for each of the records, the resources at the time interval by solving an objective function based on at least the derived weights;
recording the assigned resources and the corresponding time intervals in a log associated with health events at associated times; and
outputting the log to authorized users of the healthcare environments.

2. The computer-implemented method of claim 1, wherein constructing the model comprises generating a conflict graph with nodes representing the generated de-identified feature representations and edges representing incompatibility among the received records and the constraints.

3. The computer-implemented method of claim 1, wherein the resources comprise at least one of rooms, staff, equipment, or beds.

4. The computer-implemented method of claim 3, wherein deriving the weights comprises applying credential checks on the staff, readiness signals on the equipment, and capacity constraints on the rooms.

5. The computer-implemented method of claim 3, further comprising generating, for each of the records, a bed mask by predicting a type of the bed, predicting a length of stay, forecasting occupancy of the bed, forecasting occupancy of the room, forecasting capacity of the staff and forecasting capacity of the equipment.

6. The computer-implemented method of claim 1, further comprising:
computing, for each record, a throughput risk score by fusing de-identified clinical urgency features, the constraints, financial efficiency features, and backlog fairness features using policy-tunable weights; and
optimizing the records based on their throughput risk scores.

7. The computer-implemented method of claim 1, wherein generating the de-identified feature representation comprises:
generating de-identified identifiers from the patient information via a secure de-identification server, wherein the de-identified identifiers exclude direct patient identifiers of patient information; and
associating the de-identified feature representation with the de-identified identifier.

8. The computer-implemented method of claim 1, further comprising:
computing, for each inpatient record of the records, a discharge opportunity score based on clinical readiness, unit utilization pressure, and coordination readiness; and
determining, for each of the impatient records, whether to discharge the inpatient record based on the discharge opportunity score and a throughput risk safety mask.

9. A system for optimizing workflows in a constrained operational environment, comprising:
a processor; and
memory storing instructions that, when executed by the processor, cause the system to:
generate, within a de-identified compute plane in which computation uses de-identified identifiers and de-identified feature representations and excludes direct patient identifiers, for received operational records, a deterministic de-identified linkage identifier using a keyed function and a linkage input, and associate a de-identified state representation with the deterministic de-identified linkage identifier;
generate, within the de-identified compute plane, from at least a portion of operational attributes, a de-identified state representation that excludes direct patient identifiers and is processable within the de-identified compute plane;
compute, within the de-identified compute plane and from the de-identified state representation, a set of candidate actions available within the constrained operational environment;
compute, within the de-identified compute plane, feasibility conditions for the candidate actions based on resource availability, timing constraints, and policy-defined limits, and generate an admissibility mask identifying a subset of the candidate actions that are feasible;
gate, within the de-identified compute plane, the candidate actions using the admissibility mask to exclude infeasible candidate actions from input to optimization or learning, thereby forming a feasible action set;
apply, within the de-identified compute plane, policy-tunable weighting parameters to actions in the feasible action set and select actions using at least one of a constrained optimization process and a reinforcement learning process;
record, in an auditable decision record, at least a representation or hash of the admissibility mask used and a policy version identifier corresponding to the policy-tunable weighting parameters;
update, within the de-identified compute plane and based on observed outcomes, overrides, or deviations associated with the selected actions, at least one of feasibility conditions, the policy-tunable weighting parameters, or parameters of the constrained optimization process and/or the reinforcement learning process; and
output the selected actions as executable directives or recommendations via an interface, and permit re-identification of the deterministic de-identified linkage identifier only within a controlled re-identification enclave under an authorization policy, the controlled re-identification enclave comprising a secure user interface under role-based access control.

10. The system of claim 9, wherein the admissibility mask is enforced as a hard exclusion such that candidate actions identified as infeasible are not selectable regardless of objective-function coefficients, penalty terms, constraint relaxations, learning objectives, or policy-aware action scores.

11. The system of claim 9, wherein the constrained operational environment comprises a healthcare environment and the resources comprise at least one of rooms, staff, equipment, or beds.

12. The system of claim 9, wherein generating the de-identified state representation comprises normalizing the received operational records to a canonical schema, aligning timestamps to a scheduling time grid, removing direct patient identifiers, and transforming remaining fields into feature values that exclude individually identifiable patient information, the feature values being associated with the deterministic de-identified linkage identifier.

13. The system of claim 9, wherein the instructions further cause the system to: (a) maintain an identity-separated architecture comprising (i) the de-identified compute plane configured to operate on the de-identified state representation and the deterministic de-identified linkage identifier without access to direct patient identifiers, and (ii) a controlled re-identification enclave configured to map the deterministic de-identified linkage identifier to corresponding direct identifiers under an authorization policy; (b) prevent mapping of the deterministic de-identified linkage identifier to direct identifiers outside the controlled re-identification enclave; and (c) record, responsive to a re-identification, a re-identification event in the auditable decision record and/or in an append-only ledger.

14. The system of claim 9, wherein the admissibility mask is defined over candidate resources and discrete time intervals on a scheduling time grid and is computed from resource availability, timing constraints, and policy-defined limits.

15. The system of claim 9, wherein generating the deterministic de-identified linkage identifier comprises applying a keyed cryptographic function to the linkage input using a secret key, such that the deterministic de-identified linkage identifier is deterministic for a given key and computationally infeasible to reverse without the secret key.

16. A non-transitory computer-readable medium comprising instructions that, when executed by a processor, cause the processor to perform the following steps:
receiving data indicating one or more resources from the healthcare environments and data indicating one or more patient records from the healthcare environments;
generating, for each of the data indicating one or more of the patient records, a representation that excludes direct patient identifiers of the patient records;
constructing a model based on the generated representations, the data indicating the patient records, and the data indicating the resources;
deriving, for each of the patient records, weights from the model representing whether one or more of the resources are available for assignment at corresponding time intervals under resource constraints of the resources;
assigning, for each of the patient records, one or more of the resources at the time interval by solving an objective function based on at least the derived weights;
recording the assigned resources and the corresponding time intervals in a log associated with health events at associated times; and
outputting the log to authorized users.

17. The non-transitory computer-readable medium of claim 16, wherein outputting the log to the authorized users comprises outputting the log through a secure user interface under role-based access control.

18. The non-transitory computer-readable medium of claim 16, wherein the outputted log includes the direct patient identifiers.

19. The non-transitory computer-readable medium of claim 16, wherein the resources comprise at least one of rooms, staff, equipment, and beds.

20. The non-transitory computer-readable medium of claim 19, wherein the indicators are derived based on at least one of credential checks on the staff, readiness signals on the equipment, and capacity constraints on the rooms.
